(19) United States
(12) Patent Application Publication
(10) Pub. No.: US 2026/0218168 A1
(43) Pub. Date: Jul. 30, 2026

(54) FINE NEEDLE ASPIRATION METHODS

(71) Applicant: BioSkryb Genomics, Inc., Durham, NC (US)

(72) Inventor: Jay A.A. WEST, Durham, NC (US)

(21) Appl. No.: 19/149,817

(22) PCT Filed: Jan. 22, 2024
(86) PCT No.: PCT/US2024/012459

§ 371 Date: Jul. 21, 2025

Related U.S. Application Data
(60) Provisional application No. 63/481,130, filed on Jan. 23, 2023.

(51) Int. Cl. C12N 15/10; A61B 10/02; C12Q 1/6844; C12Q 1/6886; G01N 1/30

(52) U.S. Cl. CPC C12N 15/1096; A61B 10/0283; C12N 15/1065; C12Q 1/6844; C12Q 1/6886; G01N 1/30; C12Q 2600/156; G01N 2001/302

(57) ABSTRACT

Provided herein are compositions and methods for high-throughput Primary Template-Directed Amplification (PTA) nucleic acid amplification and sequencing methods, and their applications for mutational analysis in research, diagnostics, and treatment. Further provided herein are methods for obtaining samples using fine needle aspiration.

### CROSS-REFERENCE

This application claims the benefit of U.S. Provisional Application No. 63/481,130, filed Jan. 23, 2023, which application is incorporated herein by reference.

### BACKGROUND

Research methods that utilize nucleic amplification, e.g., Next Generation Sequencing, provide large amounts of information on complex samples, genomes, and other nucleic acid sources. In some cases, these samples are obtained in small quantities from single cells. There is a need for highly accurate, scalable, and efficient nucleic acid amplification and sequencing methods for research, diagnostics, and treatment involving small samples, especially methods for high-throughput analysis.

### BRIEF SUMMARY

Provided herein are devices, methods, and systems for spatial analysis of single cells or nuclei.

Provided herein are method of spatial nucleic acid analysis comprising: (a) providing a sample comprising a heterogeneous population of cells, wherein each cell has a unique location in the sample, and the sample was obtained using fine needle aspiration; (b) isolating one or more cells from the population of cells, wherein the location of the one or more cells is recorded; (c) amplifying DNA from one or more cells in the population of cells; and (d) generating a genotype from the DNA, wherein the location of the one or more cells and the corresponding genotype are preserved. Further provided herein are methods wherein fine needle aspiration further comprises a visualization method. Further provided herein are methods wherein the visualization method comprises one or more of ultrasound and CT scanning. Further provided herein are methods wherein the method further comprises amplifying single cells from the population of cells. Further provided herein are methods wherein the method further comprises reverse transcription of RNA in the one or more cells. Further provided herein are methods wherein the population of cells comprises mammalian cells, microbial cells, fungal cells, or plant cells. Further provided herein are methods wherein the population of cells comprises at least one cancer cell. Further provided herein are methods wherein no more than 20% of the population of cells are cancer cells. Further provided herein are methods wherein no more than 5% of the population of cells are cancer cells. Further provided herein are methods wherein no more than 1% of the population of cells are cancer cells. Further provided herein are methods wherein no more than 20% of the population of cells are isolated. Further provided herein are methods wherein no more than 5% of the population of cells are isolated. Further provided herein are methods wherein no more than 1% of the population of cells are isolated. Further provided herein are methods wherein the sample is an FFPE sample. Further provided herein are methods wherein the sample is obtained from a tissue. Further provided herein are methods wherein the tissue comprises kidney, lung, breast, brain, esophagus, pancreas, colon, skin, bladder, ovary or prostate tissue. Further provided herein are methods wherein the method further comprises scoring the one or more cells based on the genotype. Further provided herein are methods wherein the method further comprises scoring the sample based on the genotypes of one or more single cells. Further provided herein are methods wherein the one or more cells are isolated with an automated robotic device. Further provided herein are methods wherein the robotic device comprises a capillary fitting. Further provided herein are methods wherein the robotic device comprises an objective having a power of 1×-60×. Further provided herein are methods wherein the one or more cells are contacted with a stain prior to isolation. Further provided herein are methods wherein the stain is configured to identify intercellular or intracellular targets. Further provided herein are methods wherein the stain comprises Diff-Quik stain. Further provided herein are methods wherein the genotype provides for at least 95 percent alignment. Further provided herein are methods wherein the genotype provides for a presequencing library complexity of at least 3.5×10⁸ counts. Further provided herein are methods wherein the genotype provides for no more than 15% chimeras. Further provided herein are methods wherein the genotype provides for no more than 5% mitochondrial chromosome reads. Further provided herein are methods wherein amplifying DNA from one or more cells generates at least 100 ng of DNA. Further provided herein are methods wherein amplifying comprises: (a) contacting nucleic acids obtained from the isolated cells with at least one amplification primer, at least one nucleic acid polymerase, and a mixture of nucleotides, wherein the mixture of nucleotides comprises at least one terminator nucleotide which terminates nucleic acid replication by the polymerase, and (b) amplifying the nucleic acids to generate a plurality of terminated amplification products, wherein the replication proceeds by strand displacement replication and wherein the amplification is performed under conditions wherein the temperature varies by no more than 10 degrees C. Further provided herein are methods wherein the terminator is an irreversible terminator. Further provided herein are methods wherein the terminator nucleotide is selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids. Further provided herein are methods wherein the nucleotides with modification to the alpha group are alpha-thio dideoxynucleotides. Further provided herein are methods wherein the terminator nucleotide comprises modifications of the r group of the 3′ carbon of the deoxyribose. Further provided herein are methods wherein the terminator nucleotide is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. Further provided herein are methods wherein the plurality of terminated amplification products comprise an average of 1000-2000 bases in length. Further provided herein are methods wherein at least some of the amplification products comprise a cell barcode, sample barcode, or spatial location barcode.

Provided herein are methods of producing at least one map for visualizing different cell subtypes or cell states in a heterogeneous population of cells comprising: (a) providing a sample comprising a heterogeneous population of cells, wherein each cell has a unique location in the sample, and the sample was obtained using fine needle aspiration; (b) isolating one or more cells from the population of cells, wherein the location of the one or more cells is recorded; (c) amplifying DNA from one or more cells in the population of cells; (d) sequencing the amplified DNA from the one or more cells to generate a genotype; and (e) generating at least one map which correlates the location of the one or more cells with the genotype. Further provided herein are methods wherein fine needle aspiration further comprises a visualization method. Further provided herein are methods wherein the visualization method comprises one or more of ultrasound and CT scanning. Further provided herein are methods wherein the method further comprises amplifying single cells from the population of cells. Further provided herein are methods wherein the method further comprises reverse transcription of RNA in the one or more cells. Further provided herein are methods wherein the population of cells comprises mammalian cells, microbial cells, fungal cells, or plant cells. Further provided herein are methods wherein the population of cells comprises at least one cancer cell. Further provided herein are methods wherein no more than 20% of the population of cells are cancer cells. Further provided herein are methods wherein no more than 5% of the population of cells are cancer cells. Further provided herein are methods wherein no more than 1% of the population of cells are cancer cells. Further provided herein are methods wherein no more than 20% of the population of cells are isolated. Further provided herein are methods wherein no more than 5% of the population of cells are isolated. Further provided herein are methods wherein no more than 1% of the population of cells are isolated. Further provided herein are methods wherein the sample is an FFPE sample. Further provided herein are methods wherein the sample is obtained from a tissue. Further provided herein are methods wherein the tissue comprises kidney, lung, breast, brain, esophagus, pancreas, colon, skin, bladder, ovary or prostate tissue. Further provided herein are methods wherein the method further comprises scoring the one or more cells based on the genotype. Further provided herein are methods wherein the method further comprises scoring the sample based on the genotypes of one or more single cells. Further provided herein are methods wherein the one or more cells are isolated with an automated robotic device. Further provided herein are methods wherein the robotic device comprises a capillary fitting. Further provided herein are methods wherein the robotic device comprises an objective having a power of 1×-60×. Further provided herein are methods wherein the one or more cells are contacted with a stain prior to isolation. Further provided herein are methods wherein the stain is configured to identify intercellular or intracellular targets. Further provided herein are methods wherein the stain comprises Diff-Quik stain. Further provided herein are methods wherein the genotype provides for at least 95 percent alignment. Further provided herein are methods wherein the genotype provides for a presequencing library complexity of at least 3.5×10⁸ counts. Further provided herein are methods wherein the genotype provides for no more than 15% chimeras. Further provided herein are methods wherein the genotype provides for no more than 5% mitochondrial chromosome reads. Further provided herein are methods wherein amplifying DNA from one or more cells generates at least 100 ng of DNA. Further provided herein are methods wherein amplifying comprises: (a) contacting nucleic acids obtained from the isolated cells with at least one amplification primer, at least one nucleic acid polymerase, and a mixture of nucleotides, wherein the mixture of nucleotides comprises at least one terminator nucleotide which terminates nucleic acid replication by the polymerase, and (b) amplifying the nucleic acids to generate a plurality of terminated amplification products, wherein the replication proceeds by strand displacement replication and wherein the amplification is performed under conditions wherein the temperature varies by no more than 10 degrees C. Further provided herein are methods wherein the terminator is an irreversible terminator. Further provided herein are methods wherein the terminator nucleotide is selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids. Further provided herein are methods wherein the nucleotides with modification to the alpha group are alpha-thio dideoxynucleotides. Further provided herein are methods wherein the terminator nucleotide comprises modifications of the r group of the 3′ carbon of the deoxyribose. Further provided herein are methods wherein the terminator nucleotide is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. Further provided herein are methods wherein the plurality of terminated amplification products comprise an average of 1000-2000 bases in length. Further provided herein are methods wherein at least some of the amplification products comprise a cell barcode, sample barcode, or spatial location barcode.

Provided herein are systems for spatial nucleic acid analysis comprising: a sample comprising one or more cells, wherein the sample was obtained using fine needle aspiration; a device comprising: a cell collection module; an objective for visualizing single cells in the sample; and a robotic device configured to isolate single cells from the sample; and at least one reaction chamber for isothermal amplification of nucleic acids from the one or more cells with one or more terminator nucleotides. Further provided herein are systems wherein the robotic device comprises a capillary fitting. Further provided herein are systems wherein the robotic device comprises an objective having a power of 1×-60×. Further provided herein are systems wherein the system further comprises a computer interface. Further provided herein are systems wherein the system further comprises a DNA sequencing instrument. Further provided herein are systems wherein the cell collection module is configured to pick single cells, adherent colonies, or pick cells from media surface.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a schematic of multiomic analysis using Primary Template-Directed Amplification (PTA) and spatial fine needle aspirate sampling.

FIG. 2 illustrates a workflow for spatial collection of isolated single cells which undergo Primary Template-directed Amplification, followed by ligation or tagmentation-based library preparation, sequencing, and analysis with BaseJumper software.

FIG. 3A depicts CNVs detected in bulk sequencing. CNVs were only detected for IDC (invasive ductile carcinoma) samples.

FIG. 3B depicts CNV depicted in single-cell sequencing. Unlike bulk sequencing, CNVs were detected for both DCIS (ductile carcinoma in situ) and IDC (invasive ductile carcinoma) samples.

FIG. 3C depicts SNVs detected in bulk sequencing of an FFPE sample. N345K SNV was detected in single cells but not in bulk sequencing.

FIG. 3D depicts SNV depicted in single-cell sequencing.

FIG. 4A depicts spatial selection of a single cell for sequencing.

FIG. 4B depicts spatial selection of a cell cluster for sequencing.

FIG. 5 depicts H&E and α-ER staining of the primary cancer cells prior to sequencing.

FIG. 6A depicts heterogeneity in CNV in primary breast cancer cells.

FIG. 6B depicts known CNV in DCIS.

FIG. 7A depicts SNV PIK3CA mutations detected in single-cells derived from 3 separate patients.

FIG. 7B depicts SNV and CNV detected in single cells.

FIG. 8A depicts correlations between genomic and transcriptomic data.

FIG. 8B depicts correlations between genomic and transcriptomic data for a single patient 1's DCIS including CNV, expression profiles, and SNV detection.

### DETAILED DESCRIPTION OF THE INVENTION

There is a need to develop new scalable, accurate and efficient methods for spatial nucleic acid amplification and sequencing which would overcome limitations in the current methods such as physically isolating single cells, obtaining sufficient genomic material for downstream analyses, low and variable genomic coverage, amplification artifacts such as allelic bias, mutations, and chimeras. Provided herein are compositions and methods for providing accurate and scalable Primary Template-Directed Amplification (PTA) which provide for analysis of single cells from an area of a sample. Compared to other sequencing methods, the methods described herein may allow for the production of unbiased, multiomics data. Further provided herein are methods which combine fine needle aspiration with PTA multiomics.

### Definitions

Unless defined otherwise, all technical and scientific terms used herein have the same meaning as is commonly understood by one of ordinary skill in the art to which these inventions belong.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

The terms “subject” or “patient” or “individual”, as used herein, refer to animals, including mammals, such as, e.g., humans, veterinary animals (e.g., cats, dogs, cows, horses, sheep, pigs, etc.) and experimental animal models of diseases (e.g., mice, rats). In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A practical Approach, Volumes I and II (D. N. Glover ed. 1985); Oligonucleotide Synthesis (M J. Gait ed. 1984); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; B. Perbal, A practical Guide To Molecular Cloning (1984); F. M. Ausubel et al. (eds.), Current Protocols in Molecular Biology, John Wiley & Sons, Inc. (1994); among others.

The term “nucleic acid” encompasses multi-stranded, as well as single-stranded molecules. In double- or triple-stranded nucleic acids, the nucleic acid strands need not be coextensive (i.e., a double-stranded nucleic acid need not be double-stranded along the entire length of both strands). Nucleic acid templates described herein may be any size depending on the sample (from small cell-free DNA fragments to entire genomes), including but not limited to 50-300 bases, 100-2000 bases, 100-750 bases, 170-500 bases, 100-5000 bases, 50-10,000 bases, or 50-2000 bases in length. In some instances, templates are at least 50, 100, 200, 500, 1000, 2000, 5000, 10,000, 20,000 50,000, 100,000, 200,000, 500,000, 1,000,000 or more than 1,000,000 bases in length. Methods described herein provide for the amplification of nucleic acid acids, such as nucleic acid templates. Methods described herein additionally provide for the generation of isolated and at least partially purified nucleic acids and libraries of nucleic acids. In some instances, methods described herein provide for extracted nucleic acids (e.g., extracted from tissues, cells, or media). In some instances, tissue are obtained from organs. Nucleic acids include but are not limited to those comprising DNA, RNA, circular RNA, mtDNA (mitochondrial DNA), cfDNA (cell free DNA), cfRNA (cell free RNA), siRNA (small interfering RNA), cffDNA (cell free fetal DNA), mRNA, tRNA, IRNA, miRNA (microRNA), synthetic polynucleotides, polynucleotide analogues, any other nucleic acid consistent with the specification, or any combinations thereof. The length of polynucleotides, when provided, are described as the number of bases and abbreviated, such as nt (nucleotides), bp (bases), kb (kilobases), or Gb (gigabases).

The term “droplet” as used herein refers to a volume of liquid on a droplet actuator. Droplets in some instances, for example, be aqueous or non-aqueous or may be mixtures or emulsions including aqueous and non-aqueous components. For non-limiting examples of droplet fluids that may be subjected to droplet operations, see, e.g., Int. Pat. Appl. Pub. No. WO2007/120241. Any suitable system for forming and manipulating droplets can be used in the embodiments presented herein. For example, in some instances a droplet actuator is used. For non-limiting examples of droplet actuators which can be used, see, e.g., U.S. Pat. Nos. 6,911,132, 6,977,033, 6,773,566, 6,565,727, 7,163,612, 7,052,244, 7,328,979, 7,547,380, 7,641,779, U.S. Pat. Appl. Pub. Nos. US20060194331, US20030205632, US20060164490, US20070023292, US20060039823, US20080124252, US20090283407, US20090192044, US20050179746, US20090321262, US20100096266, US20110048951, Int. Pat. Appl. Pub. No. WO2007/120241. In some instances, beads are provided in a droplet, in a droplet operations gap, or on a droplet operations surface. In some instances, beads are provided in a reservoir that is external to a droplet operations gap or situated apart from a droplet operations surface, and the reservoir may be associated with a flow path that permits a droplet including the beads to be brought into a droplet operations gap or into contact with a droplet operations surface. Non-limiting examples of droplet actuator techniques for immobilizing magnetically responsive beads and/or non-magnetically responsive beads and/or conducting droplet operations protocols using beads are described in U.S. Pat. Appl. Pub. No. US20080053205, Int. Pat. Appl. Pub. No. WO2008/098236, WO2008/134153, WO2008/116221, WO2007/120241. Bead characteristics may be employed in the multiplexing embodiments of the methods described herein. Examples of beads having characteristics suitable for multiplexing, as well as methods of detecting and analyzing signals emitted from such beads, may be found in U.S. Pat. Appl. Pub. No. US20080305481, US20080151240, US20070207513, US20070064990, US20060159962, US20050277197, US20050118574.

Primers and/or template switching oligonucleotides can also be affixed to solid substrate to facilitate reverse transcription and template switching of the mRNA polynucleotides. In this arrangement a portion of the RT or template switching reaction occurs in the bulk solution of the device, where the second step of the reaction occurs in proximity to the surface. In other arrangements the primer of template switch oligonucleotide is allowed to be released from the solid substrate to allow the entire reaction to occur above the surface in the solution. In a polyomic approach the primers for the multistage reaction in some instances is affixed to the solid substrate or combined with beads to accomplish combinations of multistage primers.

Certain microfluidic devices also support polyomic approaches. Devices fabricated in PDMS, as an example, often have contiguous chambers for each reaction step. Such multichambered devices are often segregated using a microvalve structure which can be controlled through the pressure with air, or a fluid such as water or inert hydrocarbon (i.e. fluorinert). In a multiomic approach, each stage of the reaction can be sequestered and allowed to be conducted discretely. At the completion of a particular stage a valve between an adjacent chamber can be released on the substrates for the subsequent reaction can be added in a serial fashion. The result is the ability to emulate a sequential set of reactions, such as a multiomic (Protein/RNA/DNA/epigenomic) set of reactions using an individual cell as an input template material. Various microfluidics platforms may be used for analysis of single cells. Cells in some instances are manipulated through hydrodynamics (droplet microfluidics, inertial microfluidics, vortexing, microvalves, microstructures (e.g., microwells, microtraps)), electrical methods (dielectrophoresis (DEP), electroosmosis), optical methods (optical tweezers, optically induced dielectrophoresis (ODEP), opto-thermocapillary), acoustic methods, or magnetic methods. In some instances, the microfluidics platform comprises microwells. In some instances, the microfluidics platform comprises a PDMS (Polydimethylsiloxane)-based device. Non-limited examples of single cell analysis platforms compatible with the methods described herein are: ddSEQ Single-Cell Isolator, (Bio-Rad, Hercules, CA, USA, and Illumina, San Diego, CA, USA)); Chromium (10× Genomics, Pleasanton, CA, USA)); Rhapsody Single-Cell Analysis System (BD, Franklin Lakes, NJ, USA); Tapestri Platform (MissionBio, San Francisco, CA, USA)), Nadia Innovate (Dolomite Bio, Royston, UK); C1 and Polaris (Fluidigm, South San Francisco, CA, USA); ICELL8 Single-Cell System (Takara); MSND (Wafergen); Puncher platform (Vycap); CellRaft AIR System (CellMicrosystems); DEPArray NXT and DEP Array System (Menarini Silicon Biosystems); AVISO CellCelector (ALS); and InDrop System (1CellBio), and TrapTx (Celldom).

As used herein, the term “unique molecular identifier (UMI)” refers to a unique nucleic acid sequence that is attached to each of a plurality of nucleic acid molecules. When incorporated into a nucleic acid molecule, an UMI in some instances is used to correct for subsequent amplification bias by directly counting UMIs that are sequenced after amplification. The design, incorporation and application of UMIs is described, for example, in Int. Pat. Appl. Pub. No. WO 2012/142213, Islam et al. Nat. Methods (2014) 11:163-166, Kivioja, T. et al. Nat. Methods (2012) 9:72-74, Brenner et al. (2000) PNAS 97 (4), 1665, and Hollas and Schuler, (2003) Conference: 3rd International Workshop on Algorithms in Bioinformatics, Volume: 2812.

As used herein, the term “barcode” refers to a nucleic acid tag that can be used to identify a sample or source of the nucleic acid material. Thus, where nucleic acid samples are derived from multiple sources, the nucleic acids in each nucleic acid sample are in some instances tagged with different nucleic acid tags such that the source of the sample can be identified. Barcodes, also commonly referred to indexes, tags, and the like, are well known to those of skill in the art. Any suitable barcode or set of barcodes can be used. See, e.g., non-limiting examples provided in U.S. Pat. No. 8,053,192 and Int. Pat. Appl. Pub. No. WO2005/068656. Barcoding of single cells can be performed as described, for example, in U.S. Pat. Appl. Pub. No. 2013/0274117.

The terms “solid surface,” “solid support” and other grammatical equivalents herein refer to any material that is appropriate for or can be modified to be appropriate for the attachment of the primers, barcodes and sequences described herein. Exemplary substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon, nitrocellulose, ceramics, resins, silica, silica-based materials (e.g., silicon or modified silicon), carbon, metals, inorganic glasses, plastics, optical fiber bundles, and a variety of other polymers. In some embodiments, the solid support comprises a patterned surface suitable for immobilization of primers, barcodes and sequences in an ordered pattern.

As used herein, the term “biological sample” includes, but is not limited to, tissues, cells, biological fluids and isolates thereof. Cells or other samples used in the methods described herein are in some instances isolated from human patients, animals, plants, soil or other samples comprising microbes such as bacteria, fungi, protozoa, etc. In some instances, the biological sample is of human origin. In some instances, the biological is of non-human origin. The cells in some instances undergo PTA methods described herein and sequencing. Variants detected throughout the genome or at specific locations can be compared with all other cells isolated from that subject to trace the history of a cell lineage for research or diagnostic purposes. In some instances, variants are confirmed through additional methods of analysis such as direct PCR sequencing.

Fine needle aspiration (FNA)

Described herein are devices, methods, systems, and compositions for analysis of single cells within a sample. In some instances, workflows comprise use of the PTA method. In some instances, cells are obtained using fine needle aspiration (FNA). In some instances, FNA comprises directing a needle along a pre-determined vector into a tissue of a subject, and cells and/or fluid is removed for sampling. In some instances, the needle size is 27-14 gauge. In some instances, the needle size is 25-18 gauge. In some instances, the needle is size is 27, 26, 25, 24, 23, 22, 21, 20, 19, 18, 17, 16, 15, or 14 gauge. In some instances, the needle is size is at least 27, 26, 25, 24, 23, 22, 21, 20, 19, 18, 17, 16, 15, or 14 gauge. In some instances, the needle is size is at most 27, 26, 25, 24, 23, 22, 21, 20, 19, 18, 17, 16, 15, or 14 gauge. In some instances, the needle size is 27-18, 27-20, 27-22, 27-25, 25-14, 25-18, 25-20, 25-22, 22-14, 22-18, 22-20, 20-14, 20-18, or 18-14 gauge. The needle size may be based on the type of tissue from which the cells and/or fluid is removed. FNA in some instances is used to obtain tissue samples from the kidney, lung, breast, brain, thyroid, lymph nodes, esophagus, pancreas, colon, skin, bladder, skin, ovary or prostate tissue. In some instances, FNA is used in conjunction with a visualization method. In some instances, the visualization method is used to orient the needle vector, and ensure the needle is correctly guided to the desired region at a location in the subject. In some instances, the needle is correctly guided to more than one region at locations in the subject. Visualization methods in some instances include one or more of CT scanning, MRI, fluoroscopy, and ultrasound. In some instances, one or more FNA samples are taken from the same area. In some instances, one or more FNA samples are taken from a different area/region of the same tissue. In some instances, at least one, two, three, four, five, or more than five FNA samples are obtained from the same area. After sampling, the sample in some instances is placed on a sampling compartment or surface for single cell picking/isolation. In some instances, cells are alcohol-fixed prior to isolation. In some instances, the sample is stained. In some instances, the stain comprises Diff-Quik. In some instances, the stain comprises Papanicolaou stain. In some instances, the stain comprises Hematoxylin and Eosin (H&E) stain. In some instances, the stain comprises a Romanowsky stain (Panoptic). In some instances, the stain comprises May-Grünwald-Giemsa stain. In some instances, structure that are stained comprise one or more of erythrocytes, platelets, neutrophils, eosinophils, basophils, monocytes, bacteria/fungi, cytoplasm/collagen/muscle, and spermatozoa.

### Spatial Nucleic Acid Analysis

Described herein are devices, methods, systems, and compositions for analysis of single cells within a sample. In some instances, workflows comprise use of the PTA method. Cellular Heterogeneity dictates the fate of all tissues in both normal development and the pathogenesis of human disease. Defining this heterogeneity has primarily been focused on the expression of discrete genes in single cells. While expression based analysis is highly valuable for defining variable cell populations, actionable information on therapeutic selection for oncology in some instances depends on the highest resolution genome data possible. One limitation of attaining high definition genome data from single cells been gated by both the ability to isolate the important cells of interest and the ability to amplify the genome with high uniformity and complete coverage. By combining the ability to select individual cells from a surface, transfer these cells to a reaction vessel, such as a microtiter plate, provided herein is a workflow to spatially locate a cell of interest, capture that individual cell and amplify the genome to enable NGS analysis of the genome with high data quality. In some instances, the combined system allows the definition of genomic heterogeneity by the spatial selection of cells followed by PTA. In some instances, cells are dissociated.

Provided herein are devices, systems and methods for isolation of cells. In some instances, methods comprise image based single-cell selection. In some instances, selection of suspension and adherent cells is used. In some instances, single cells are selected from samples containing more than 10K cells. In some instances, cells are isolated using a fine needle aspirate. In some instances, cells are isolated from one or more tissue samples using a fine needle aspirate. Obtaining cells using a fine needle aspirate can allow for minimally invasive isolation of cells from one or more regions of tissue with high accuracy. In some instances, cells are selected from microwell well plates, slides, and nanowell arrays, and polymer gels (colony picking). In some embodiments, immunomagnetically enriched cells are localized by a magnetic field. In some instances, selected suspension or adherent single cells are then captured and automatically placed in discrete volume in a collection tube or a 24, 96, 384, or 1536 well plate. In some instances, nucleic acids are then isolated from cells, and subjected to amplification with the PTA method. In some instances, the samples obtained using FNA may have low cellularity (e.g., excessive blood or fluid diluting the sample). In some instances, PTA can overcome this shortcoming by yielding high quality results from few cells, as further described herein, such that there is little to no effect on the accuracy or quality of the results. This may also reduce the burden on patients and healthcare professionals such that they do not have to repeat procedures. After library generation from PTA-created amplicons, nucleic acids are sequenced. In some instances, more than 80%, 90%, 95%, 97%, 98%, or 99% of the genome is captured by sequencing. In some instances, PTA results in high breadth of coverage, few replication errors, and low allelic bias, to accurately call single nucleotide variants (SNV) at the whole genome sequencing (WGS), whole exome sequencing (WES), and small-panel levels. In some instances, input DNA from single cells is no more than 500, 400, 300, 200, 150, 100, 75, or 50 ng of unfragmented DNA. In some instances, libraries are created using tagmentation-based workflows.

Heterogeneity within cellular populations in some instances drives the underlying biology of life. Single-cell DNA sequencing offers the ability to discern the mechanisms with the highest resolution how each cell modifies their genomes (and resultant transcriptome and proteomes) during development and the advancement of pathology. Using the ability to select and isolate cells from a specific positional location the spatial and temporal nature of the genomic signature that influences tissue organization can be associated. At a fundamental level for oncology, the ability to select cells from specific location or region within a tissue, and analyze each discrete genome will allow the understanding of tumor heterogeneity in a spatial and temporal context. This evolution from the founding clone, through a pre-cancerous lesion, to a oncology based pathology is indeed rooted not only in the menagerie of various cells, but in the way they interact with each other, the tissue ultrastructure as well as the circulatory and lymphatic systems.

### Single Cell Multiomics

Described herein are methods, devices, and compositions for high-throughput analysis of single cells. Analysis of cells in bulk provides general information about the cell population, but often is unable to detect low-frequency mutants over the background. Such mutants may comprise important properties such as drug resistance or mutations associated with cancer. In some instances, DNA, RNA, and/or proteins from the same single cell are analyzed in parallel, using the devices described herein. The analysis may include identification of epigenetic post-translational (e.g., glycosylation, phosphorylation, acetylation, ubiquitination, histone modification) and/or post-transcriptional (e.g., methylation, hydroxymethylation) modifications. Such methods may comprise “Primary Template-Directed Amplification” (PTA) to obtain libraries of nucleic acids for sequencing. In some instances PTA is combined with additional steps or methods such as RT-PCR or proteome/protein quantification techniques (e.g., mass spectrometry, antibody staining, etc.). In some instances, various components of a cell are physically or spatially separated from each other during individual analysis steps. In some instances, proteins are first labeled with antibodies. In some instances, at least some of the antibodies comprise a tag or marker (e.g., nucleic acid/oligo tag, mass tag, or fluorescent tag). In some instances, a portion of the antibodies comprise an oligo tag. In some instances, a portion of the antibodies comprise a fluorescent marker. In some instances, antibodies are labeled by two or more tags or markers. In some instances, a portion of the antibodies are sorted based on fluorescent markers. In some embodiments, one or more cells are contacted with a stain prior to isolation. A stain may include, without limitations, an antibody, an oligonucleotide, or a substrate-based reaction. A stain may be visualized by a dye. The dye may be a fluorescent dye or a non-fluorescent dye. The stain allows detection of a specific analyte. After reverse transcription, first strand mRNA products are generated and then removed for analysis. Libraries are then generated from first strand reaction products and barcodes present on protein-specific antibodies, which are subsequently sequenced. In parallel, genomic DNA from the same cell is subjected to PTA, a library generated, and sequenced. Sequencing results from the genome, proteome, and transcriptome are in some instances pooled using bioinformatics methods. Methods described herein in some instances comprise any combination of labeling, cell sorting, affinity separation/purification, lysing of specific cell components (e.g., outer membrane, nucleus, etc.), RNA amplification, DNA amplification (e.g., PTA), or other step associated with protein, RNA, or DNA isolation or analysis. In some instances, methods described herein comprise one or more enrichment steps, such as exome enrichment. In some instances, multiomics comprises measurements of one or more of genome (CNV, translocation, mutation burden), methylome (methylation at CpG sites, gene activation, gene repression), transcriptome (expressed genes, gene fusions, splice variants), and proteome (translation level, phosphorylation state, and protein modification).

Described herein is a first method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. Alternatively or in combination, centrifugation is used to separate RNA in the supernatant from cDNA in the cell pellet. Remaining cDNA is in some instances fragmented and removed with UDG (uracil DNA glycosylase), and alkaline lysis is used to degrade RNA and denature the genome. After neutralization, addition of primers and PTA, amplification products are in some instances purified on SPRI (solid phase reversible immobilization) beads, and ligated to adapters to generate a gDNA library.

Described herein is a second method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. In some instances, alkaline lysis is then used to degrade RNA and denature the genome. After neutralization, addition of random primers and PTA, amplification products are in some instances purified on SPRI (solid phase reversible immobilization) beads, and ligated to adapters to generate a gDNA library. RT products are in some instances isolated by pulldown, such as a pulldown with streptavidin beads.

Described herein is a third method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs) in the presence of terminator nucleotides. In some instances, TSOs comprise a molecular TAG such as biotin, which allows subsequent pull-down of cDNA RT products, and PCR amplification of RT products to generate a cDNA library. In some instances, alkaline lysis is then used to degrade RNA and denature the genome. After neutralization, addition of random primers and PTA, amplification products are in some instances purified on SPRI (solid phase reversible immobilization) beads, and ligated to adapters to generate a DNA library. RT products are in some instances isolated by pulldown, such as a pulldown with streptavidin beads.

Described herein is a fourth method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. In some instances, alkaline lysis is then used to degrade RNA and denature the genome. After neutralization, addition of random primers and PTA, amplification products are in some instances subjected to RNase and cDNA amplification using blocked and labeled primers. gDNA is purified on SPRI (solid phase reversible immobilization) beads, and ligated to adapters to generate a gDNA library. RT products are in some instances are isolated by pulldown, such as a pulldown with streptavidin beads.

Described herein is a fifth method of single cell analysis comprising analysis of RNA and DNA from a single cell. A population of cells is contacted with an antibody library, wherein antibodies are labeled. In some instances, antibodies are labeled with either fluorescent labels, nucleic acid barcodes, or both. Labeled antibodies bind to at least one cell in the population, and such cells are sorted, placing one cell per container (e.g., a tube, vial, microwell, etc.). In some instances, the container comprises a solvent. In some instances, a region of a surface of a container is coated with a capture moiety. In some instances, the capture moiety is a small molecule, an antibody, a protein, or other agent capable of binding to one or more cells, organelles, or other cell component. In some instances, at least one cell, or a single cell, or component thereof, binds to a region of the container surface. In some instances, a nucleus binds to the region of the container. In some instances, the outer membrane of the cell is lysed, releasing mRNA into a solution in the container. In some instances, the nucleus of the cell containing genomic DNA is bound to a region of the container surface. Next, RT is often performed using the mRNA in solution as a template to generate cDNA. In some instances, template switching primers comprise from 5′ to 3′ a TSS region (transcription start site), an anchor region, a RNA BC region, and a poly dT tail. In some instances, the poly dT tail binds to poly A tail of one or more mRNAs. In some instances, template switching primers comprise from 3′ to 5′ a TSS region, an anchor region, and a poly G region. In some instances, the poly G region comprises riboG. In some instances the poly G region binds to a poly C region on an mRNA transcript. In some instances, riboG was added to the mRNA transcripts by a terminal transferase. After removal of RT PCR products for subsequent sequencing, any remaining RNA in the cell is removed by UNG. The nucleus is then lysed, and the released genomic DNA is subjected to the PTA method using random primers with an isothermal polymerase. In some instances, primers are 6-9 bases in length. In some instances, PTA generates genomic amplicons of 100-5000, 200-5000, 500-2000, 500-2500, 1000-3000, or 300-3000 bases in length. In some instances, PTA generates genomic amplicons with an average length of 100-5000, 200-5000, 500-2000, 500-2500, 1000-3000, or 300-3000 bases. In some instances, PTA generates genomic amplicons of 250-1500 bases in length. In some instances, the methods described herein generate a short fragment cDNA pool with about 500, about 750, about 1000, about 5000, or about 10,000 fold amplification. In some instances, the methods described herein generate a short fragment cDNA pool with 500-5000, 750-1500, or 250-10,000 fold amplification. PTA products are optionally subjected to additional amplification and sequenced.

### Sample Preparation and Isolation of Single Cells

Methods described herein may require isolation of single cells for analysis. Any method of single cell isolation may be used with PTA, such as mouth pipetting, micro pipetting, flow cytometry/FACS, microfluidics, methods of sorting nuclei (tetraploid or other), or manual dilution. Such methods are aided by additional reagents and steps, for example, antibody-based enrichment (e.g., circulating tumor cells), other small-molecule or protein-based enrichment methods, or fluorescent labeling. In some instances, a method of multiomic analysis described herein comprises mechanical or enzymatic dissociate of cells from larger tissues.

### Preparation and Analysis of Cell Components

Methods of multiomic analysis comprising PTA described herein may comprise one or more methods of processing cell components such as DNA, RNA, and/or proteins. In some instances, the nucleus (comprising genomic DNA) is physically separated from the cytosol (comprising mRNA), followed by a membrane-selective lysis buffer to dissolve the membrane but keep the nucleus intact. The cytosol is then separated from the nucleus using methods including micro pipetting, centrifugation, or anti-body conjugated magnetic microbeads. In another instance, an oligo-dT primer coated magnetic bead binds polyadenylated mRNA for separation from DNA. In another instance, DNA and RNA are preamplified simultaneously, and then separated for analysis. In another instance, a single cell is split into two equal pieces, with mRNA from one half processed, and genomic DNA from the other half processed.

### Multiomics

Methods described herein (e.g., PTA) may be used as a replacement for any number of other known methods in the art which are used for single cell sequencing (multiomics or the like). PTA may substitute genomic DNA sequencing methods such as MDA, PicoPlex, DOP-PCR, MALBAC, or target-specific amplifications. In some instances, PTA replaces the standard genomic DNA sequencing method in a multiomics method including DR-seq (Dey et al., 2015), G&T seq (MacAulay et al., 2015), scMT-seq (Hu et al., 2016), sc-GEM (Cheow et al., 2016), scTrio-seq (Hou et al., 2016), simultaneous multiplexed measurement of RNA and proteins (Darmanis et al., 2016), scCOOL-seq (Guo et al., 2017), CITE-seq (Stoeckius et al., 2017), REAP-seq (Peterson et al., 2017), scNMT-seq (Clark et al., 2018), or SIDR-seq (Han et al., 2018). In some instances, a method described herein comprises PTA and a method of polyadenylated mRNA transcripts. In some instances, a method described herein comprises PTA and a method of non-polyadenylated mRNA transcripts. In some instances, a method described herein comprises PTA and a method of total (polyadenylated and non-polyadenylated) mRNA transcripts.

In some instances, PTA is combined with a standard RNA sequencing method to obtain genome and transcriptome data. In some instances, a multiomics method described herein comprises PTA and one of the following: Drop-seq (Macosko, et al. 2015), mRNA-seq (Tang et al., 2009), InDrop (Klein et al., 2015), MARS-seq (Jaitin et al., 2014), Smart-seq2 (Hashimshony, et al., 2012; Fish et al., 2016), CEL-seq (Jaitin et al., 2014), STRT-seq (Islam, et al., 2011), Quartz-seq (Sasagawa et al., 2013), CEL-seq2 (Hashimshony, et al. 2016), cytoSeq (Fan et al., 2015), SuPeR-seq (Fan et al., 2011), RamDA-seq (Hayashi, et al. 2018), MATQ-seq (Sheng et al., 2017), or SMARTer (Verboom et al., 2019).

Various reaction conditions and mixes may be used for generating cDNA libraries for transcriptome analysis. In some instances, an RT reaction mix is used to generate a cDNA library. In some instances, the RT reaction mixture comprises a crowding reagent, at least one primer, a template switching oligonucleotide (TSO), a reverse transcriptase, and a dNTP mix. In some instances, an RT reaction mix comprises an RNAse inhibitor. In some instances, an RT reaction mix comprises one or more surfactants. In some instances an RT reaction mix comprises Tween-20 and/or Triton-X. In some instances an RT reaction mix comprises Betaine. In some instances an RT reaction mix comprises one or more salts. In some instances an RT reaction mix comprises a magnesium salt (e.g., magnesium chloride) and/or tetramethylammonium chloride. In some instances an RT reaction mix comprises gelatin. In some instances an RT reaction mix comprises PEG (PEG1000, PEG2000, PEG4000, PEG6000, PEG8000, or PEG of other length).

Multiomic methods described herein may provide both genomic and RNA transcript information from a single cell (e.g., a combined or dual protocol). In some instances, genomic information from the single cell is obtained from the PTA method, and RNA transcript information is obtained from reverse transcription to generate a cDNA library. In some instances, a whole transcript method is used to obtain the cDNA library. In some instances, 3′ or 5′ end counting is used to obtain the cDNA library. In some instances, cDNA libraries are not obtained using UMIs. In some instances, a multiomic method provides RNA transcript information from the single cell for at least 500, 1000, 2000, 5000, 7000, 8000, 10,000, 12,000, or at least 15,000 genes. In some instances, a multiomic method provides RNA transcript information from the single cell for about 500, 1000, 2000, 5000, 7000, 8000, 10,000, 12,000, or about 15,000 genes. In some instances, a multiomic method provides RNA transcript information from the single cell for 100-12,000 1000-10,000, 2000-15,000, 5000-15,000, 10,000-20,000, 8000-15,000, or 10,000-15,000 genes. In some instances, a multiomic method provides genomic sequence information for at least 80%, 90%, 92%, 95%, 97%, 98%, or at least 99% of the genome of the single cell. In some instances, a multiomic method provides genomic sequence information for about 80%, 90%, 92%, 95%, 97%, 98%, or about 99% of the genome of the single cell.

Multiomic methods may comprise analysis of single cells from a population of cells. In some instances, at least 5, 10, 20, 50, 100, 200, 500, 1000, 2000, 5000, or at least 8000 cells are analyzed. In some instances, about 5, 10, 20, 50, 100, 200, 500, 1000, 2000, 5000, or about 8000 cells are analyzed. In some instances, 5-100, 10-100, 50-500, 100-500, 100-1000, 50-5000, 100-5000, 500-1000, 500-10000, 1000-10000, or 5000-20,000 cells are analyzed.

Multiomic methods may generate yields of genomic DNA from the PTA reaction based on the type of single cell. In some instances, the amount of DNA generated from a single cell is about 0.1, 1, 1.5, 2, 3, 5, or about 10 micrograms. In some instances, the amount of DNA generated from a single cell is about 0.1, 1, 1.5, 2, 3, 5, or about 10 femtograms. In some instances, the amount of DNA generated from a single cell is at least 0.1, 1, 1.5, 2, 3, 5, or at least 10 micrograms. In some instances, the amount of DNA generated from a single cell is at least 0.1, 1, 1.5, 2, 3, 5, or at least 10 femtograms. In some instances, the amount of DNA generated from a single cell is about 0.1-10, 1-10, 1.5-10, 2-20, 2-50, 1-3, or 0.5-3.5 micrograms. In some instances, the amount of DNA generated from a single cell is about 0.1-10, 1-10, 1.5-10, 2-20, 2-4, 1-3, or 0.5-4 femtograms.

### Methylome Analysis

Described herein are methods comprising PTA, wherein sites of methylated DNA in single cells are determined using the PTA method. In some instances, these methods further comprise parallel analysis of the transcriptome and/or proteome of the same cell. Methods of detecting methylated genomic bases include selective restriction with methylation-sensitive endonucleases, followed by processing with the PTA method. Sites cut by such enzymes are determined from sequencing, and methylated bases are identified. In another instance, bisulfite treatment of genomic DNA libraries converts unmethylated cytosines to uracil. Libraries are then in some instances amplified with methylation-specific primers which selectively anneal to methylated sequences. Alternatively, non-methylation-specific PCR is conducted, followed by one or more methods to discriminate between bisulfite-reacted bases, including direct pyrosequencing, MS-SnuPE, HRM, COBRA, MS-SSCA, or base-specific cleavage/MALDI-TOF. In some instances, genomic DNA samples are split for parallel analysis of the genome (or an enriched portion thereof) and methylome analysis. In some instances, analysis of the genome and methylome comprises enrichment of genomic fragments (e.g., exome, or other targets) or whole genome sequencing.

### Bioinformatics

The data obtained from single-cell analysis methods utilizing PTA described herein may be compiled into a database. Described herein are methods and systems of bioinformatic data integration. Data from the proteome, genome, transcriptome, methylome or other data is in some instances combined/integrated into a database and analyzed. Bioinformatic data integration methods and systems in some instances comprise one or more of protein detection (FACS and/or NGS), mRNA detection, and/or genome variance detection. In some instances, this data is correlated with a disease state or condition. In some instances, data from a plurality of single cells is compiled to describe properties of a larger cell population, such as cells from a specific sample, region, organism, or tissue. In some instances, protein data is acquired from fluorescently labeled antibodies which selectively bind to proteins on a cell. In some instances, a method of protein detection comprises grouping cells based on fluorescent markers and reporting sample location post-sorting. In some instances, a method of protein detection comprises detecting sample barcodes, detecting protein barcodes, comparing to designed sequences, and grouping cells based on barcode and copy number. In some instances, the protein structure is inferred by the genome sequence of the cell. In some instances, protein data is acquired from barcoded antibodies which selectively bind to proteins on a cell. In some instances, transcriptome data is acquired from sample and RNA specific barcodes. In some instances, a method of mRNA detection comprises detecting sample and RNA specific barcodes, aligning to genome, aligning to RefSeq/Encode, reporting Exon/Intro/Intergenic sequences, analyzing exon-exon junctions, grouping cells based on barcode and expression variance and clustering analysis of variance and top variable genes. In some instances, genomic data is acquired from sample and DNA specific barcodes. In some instances, a method of genome variance detection comprises detecting sample and DNA specific barcodes, aligning to the genome, determine genome recovery and SNV mapping rate, filtering reads on exon-exon junctions, generating variant call file (VCF), and clustering analysis of variance and top variable mutations.

### Mutations

In some instances, the methods (e.g., multiomic PTA) described herein result in higher detection sensitivity and/or lower rates of false positives for the detection of mutations. In some instances a mutation is a difference between an analyzed sequence (e.g., using the methods described herein) and a reference sequence. Reference sequences are in some instances obtained from other organisms, other individuals of the same or similar species, populations of organisms, or other areas of the same genome. In some instances, mutations are identified on a plasmid or chromosome. In some instances, a mutation is an SNV (single nucleotide variation), SNP (single nucleotide polymorphism), or CNV (copy number variation, or CNA/copy number aberration). In some instances, a mutation is base substitution, insertion, or deletion. In some instances, a mutation is a transition, transversion, nonsense mutation, silent mutation, synonymous or non-synonymous mutation, non-pathogenic mutation, missense mutation, or frameshift mutation (deletion or insertion). In some instances, PTA results in higher detection sensitivity and/or lower rates of false positives for the detection of mutations when compared to methods such as in-silico prediction, ChIP-seq, GUIDE-seq, circle-seq, HTGTS (High-Throughput Genome-Wide Translocation Sequencing), IDLV (integration-deficient lentivirus), Digenome-seq, FISH (fluorescence in situ hybridization), or DISCOVER-seq.

### Primary Template-Directed Amplification

Described herein are nucleic acid amplification methods, such as “Primary Template-Directed Amplification (PTA).” In some instances, PTA is combined with other analysis workflows for multiomic analysis. With the PTA method, amplicons are preferentially generated from the primary template (“direct copies”) using a polymerase (e.g., a strand displacing polymerase). Consequently, errors are propagated at a lower rate from daughter amplicons during subsequent amplifications compared to MDA. The result is an easily executed method that, unlike existing WGA protocols, can amplify low DNA input including the genomes of single cells with high coverage breadth and uniformity in an accurate and reproducible manner. In some instances, PTA enables kinetic control of an amplification reaction. In some instances, PTA results in a pseudo-linear amplification reaction (rather than exponential amplification). Moreover, the terminated amplification products can undergo direction ligation after removal of the terminators, allowing for the attachment of a cell barcode to the amplification primers so that products from all cells can be pooled after undergoing parallel amplification reactions. In some instances, template nucleic acids are not bound to a solid support. In some instances, direct copies of template nucleic acids are not bound to a solid support. In some instances, one or more primers are not bound to a solid support. In some instances, no primers are not bound to a solid support. In some instances, a primer is attached to a first solid support, and a template nucleic acid is attached to a second solid support, wherein the first and the second solid supports are not the same. In some instances, PTA is used to analyze single cells from a larger population of cells. In some instances, PTA is used to analyze more than one cell from a larger population of cells, or an entire population of cells.

Described herein are methods employing nucleic acid polymerases with strand displacement activity for amplification. In some instances, such polymerases comprise strand displacement activity and low error rate. In some instances, such polymerases comprise strand displacement activity and proofreading exonuclease activity, such as 3′->5′ proofreading activity. In some instances, nucleic acid polymerases are used in conjunction with other components such as reversible or irreversible terminators, or additional strand displacement factors. In some instances, the polymerase has strand displacement activity, but does not have exonuclease proofreading activity. For example, in some instances such polymerases include bacteriophage phi29 (Φ29) polymerase, which also has very low error rate that is the result of the 3′->5′ proofreading exonuclease activity (see, e.g., U.S. Pat. Nos. 5,198,543 and 5,001,050). In some instances, non-limiting examples of strand displacing nucleic acid polymerases include, e.g., genetically modified phi29 (Φ29) DNA polymerase, Klenow Fragment of DNA polymerase I (Jacobsen et al., Eur. J. Biochem. 45:623-627 (1974)), phage M2 DNA polymerase (Matsumoto et al., Gene 84:247 (1989)), phage phiPRD1 DNA polymerase (Jung et al., Proc. Natl. Acad. Sci. USA 84:8287 (1987); Zhu and Ito, Biochim. Biophys. Acta. 1219:267-276 (1994)), Bst DNA polymerase (e.g., Bst large fragment DNA polymerase (Exo(-) Bst; Aliotta et al., Genet. Anal. (Netherlands) 12:185-195 (1996)), exo(-) Bca DNA polymerase (Walker and Linn, Clinical Chemistry 42:1604-1608 (1996)), Bsu DNA polymerase, Vent_{R} DNA polymerase including Vent_{R} (exo-) DNA polymerase (Kong et al., J. Biol. Chem. 268:1965-1975 (1993)), Deep Vent DNA polymerase including Deep Vent (exo-) DNA polymerase, IsoPol DNA polymerase, DNA polymerase I, Therminator DNA polymerase, T5 DNA polymerase (Chatterjee et al., Gene 97:13-19 (1991)), Sequenase (U.S. Biochemicals), T7 DNA polymerase, T7-Sequenase, T7 gp5 DNA polymerase, PRDI DNA polymerase, T4 DNA polymerase (Kaboord and Benkovic, Curr. Biol. 5:149-157 (1995)). Additional strand displacing nucleic acid polymerases are also compatible with the methods described herein. The ability of a given polymerase to carry out strand displacement replication can be determined, for example, by using the polymerase in a strand displacement replication assay (e.g., as disclosed in U.S. Pat. No. 6,977,148). Such assays in some instances are performed at a temperature suitable for optimal activity for the enzyme being used, for example, 32° C. for phi29 DNA polymerase, from 46° C. to 64° C. for exo(-) Bst DNA polymerase, or from about 60° C. to 70° C. for an enzyme from a hyperthermophylic organism. Another useful assay for selecting a polymerase is the primer-block assay described in Kong et al., J. Biol. Chem. 268:1965-1975 (1993). The assay consists of a primer extension assay using an M13 ssDNA template in the presence or absence of an oligonucleotide that is hybridized upstream of the extending primer to block its progress. Other enzymes capable of displacement the blocking primer in this assay are in some instances useful for the disclosed method. In some instances, polymerases incorporate dNTPs and terminators at approximately equal rates. In some instances, the ratio of rates of incorporation for dNTPs and terminators for a polymerase described herein are about 1:1, about 1.5:1, about 2:1, about 3:1 about 4:1 about 5:1, about 10:1, about 20:1 about 50:1, about 100:1, about 200:1, about 500:1, or about 1000:1. In some instances, the ratio of rates of incorporation for dNTPs and terminators for a polymerase described herein are 1:1 to 1000:1, 2:1 to 500:1, 5:1 to 100:1, 10:1 to 1000:1, 100:1 to 1000:1, 500:1 to 2000:1, 50:1 to 1500:1, or 25:1 to 1000:1.

Described herein are methods of amplification wherein strand displacement can be facilitated through the use of a strand displacement factor, such as, e.g., helicase. Such factors are in some instances used in conjunction with additional amplification components, such as polymerases, terminators, or other component. In some instances, a strand displacement factor is used with a polymerase that does not have strand displacement activity. In some instances, a strand displacement factor is used with a polymerase having strand displacement activity. Without being bound by theory, strand displacement factors may increase the rate that smaller, double stranded amplicons are reprimed. In some instances, any DNA polymerase that can perform strand displacement replication in the presence of a strand displacement factor is suitable for use in the PTA method, even if the DNA polymerase does not perform strand displacement replication in the absence of such a factor. Strand displacement factors useful in strand displacement replication in some instances include (but are not limited to) BMRF1 polymerase accessory subunit (Tsurumi et al., J. Virology 67 (12): 7648-7653 (1993)), adenovirus DNA-binding protein (Zijderveld and van der Vliet, J. Virology 68 (2): 1158-1164 (1994)), herpes simplex viral protein ICP8 (Boehmer and Lehman, J. Virology 67 (2): 711-715 (1993); Skaliter and Lehman, Proc. Natl. Acad. Sci. USA 91 (22): 10665-10669 (1994)); single-stranded DNA binding proteins (SSB; Rigler and Romano, J. Biol. Chem. 270:8910-8919 (1995)); phage T4 gene 32 protein (Villemain and Giedroc, Biochemistry 35:14395-14404 (1996); T7 helicase-primase; T7 gp2.5 SSB protein; Tte-UvrD (from Thermoanaerobacter tengcongensis), calf thymus helicase (Siegel et al., J. Biol. Chem. 267:13629-13635 (1992)); bacterial SSB (e.g., E. coli SSB), Replication Protein A (RPA) in eukaryotes, human mitochondrial SSB (mtSSB), and recombinases, (e.g., Recombinase A (RecA) family proteins, T4 UvsX, T4 UvsY, Sak4 of Phage HK620, Rad51, Dmc1, or Radb). Combinations of factors that facilitate strand displacement and priming are also consistent with the methods described herein. For example, a helicase is used in conjunction with a polymerase. In some instances, the PTA method comprises use of a single-strand DNA binding protein (SSB, T4 gp32, or other single stranded DNA binding protein), a helicase, and a polymerase (e.g., SauDNA polymerase, Bsu polymerase, Bst2.0, GspM, GspM2.0, GspSSD, or other suitable polymerase). In some instances, reverse transcriptases are used in conjunction with the strand displacement factors described herein. In some instances, reverse transcriptases are used in conjunction with the strand displacement factors described herein. In some instances, amplification is conducted using a polymerase and a nicking enzyme (e.g., “NEAR”), such as those described in U.S. Pat. No. 9,617,586. In some instances, the nicking enzyme is Nt.BspQI, Nb.BbvCi, Nb.BsmI, Nb.BsrDI, Nb.BtsI, Nt.AlwI, Nt.BbvCI, Nt.BstNBI, Nt. CviPII, Nb.Bpu10I, or Nt.Bpu10I.

Described herein are amplification methods comprising use of terminator nucleotides, polymerases, and additional factors or conditions. For example, such factors are used in some instances to fragment the nucleic acid template(s) or amplicons during amplification. In some instances, such factors comprise endonucleases. In some instances, factors comprise transposases. In some instances, mechanical shearing is used to fragment nucleic acids during amplification. In some instances, nucleotides are added during amplification that may be fragmented through the addition of additional proteins or conditions. For example, uracil is incorporated into amplicons; treatment with uracil D-glycosylase fragments nucleic acids at uracil-containing positions. Additional systems for selective nucleic acid fragmentation are also in some instances employed, for example an engineered DNA glycosylase that cleaves modified cytosine-pyrene base pairs. (Kwon, et al. Chem Biol. 2003, 10(4), 351)

Described herein are amplification methods comprising use of terminator nucleotides, which terminate nucleic acid replication thus decreasing the size of the amplification products. Such terminators are in some instances used in conjunction with polymerases, strand displacement factors, or other amplification components described herein. In some instances, terminator nucleotides reduce or lower the efficiency of nucleic acid replication. Such terminators in some instances reduce extension rates by at least 99.9%, 99%, 98%, 95%, 90%, 85%, 80%, 75%, 70%, or at least 65%. Such terminators in some instances reduce extension rates by 50%-90%, 60%-80%, 65%-90%, 70%-85%, 60%-90%, 70%-99%, 80%-99%, or 50%-80%. In some instances terminators reduce the average amplicon product length by at least 99.9%, 99%, 98%, 95%, 90%, 85%, 80%, 75%, 70%, or at least 65%. Terminators in some instances reduce the average amplicon length by 50%-90%, 60%-80%, 65%-90%, 70%-85%, 60%-90%, 70%-99%, 80%-99%, or 50%-80%. In some instances, amplicons comprising terminator nucleotides form loops or hairpins which reduce a polymerase's ability to use such amplicons as templates. Use of terminators in some instances slows the rate of amplification at initial amplification sites through the incorporation of terminator nucleotides (e.g., dideoxynucleotides that have been modified to make them exonuclease-resistant to terminate DNA extension), resulting in smaller amplification products. By producing smaller amplification products than the currently used methods (e.g., average length of 50-2000 nucleotides in length for PTA methods as compared to an average product length of >10,000 nucleotides for MDA methods) PTA amplification products in some instances undergo direct ligation of adapters without the need for fragmentation, allowing for efficient incorporation of cell barcodes and unique molecular identifiers (UMI).

Terminator nucleotides are present at various concentrations depending on factors such as polymerase, template, or other factors. For example, the amount of terminator nucleotides in some instances is expressed as a ratio of non-terminator nucleotides to terminator nucleotides in a method described herein. Such concentrations in some instances allow control of amplicon lengths. In some instances, the ratio of terminator to non-terminator nucleotides is modified for the amount of template present or the size of the template. In some instances, the ratio of ratio of terminator to non-terminator nucleotides is reduced for smaller samples sizes (e.g., femtogram to picogram range). In some instances, the ratio of non-terminator to terminator nucleotides is about 2:1, 5:1, 7:1, 10:1, 20:1, 50:1, 100:1, 200:1, 500:1, 1000:1, 2000:1, or 5000:1. In some instances the ratio of non-terminator to terminator nucleotides is 2:1-10:1, 5:1-20:1, 10:1-100:1, 20:1-200:1, 50:1-1000:1, 50:1-500:1, 75:1-150:1, or 100:1-500:1. In some instances, at least one of the nucleotides present during amplification using a method described herein is a terminator nucleotide. Each terminator need not be present at approximately the same concentration; in some instances, ratios of each terminator present in a method described herein are optimized for a particular set of reaction conditions, sample type, or polymerase. Without being bound by theory, each terminator may possess a different efficiency for incorporation into the growing polynucleotide chain of an amplicon, in response to pairing with the corresponding nucleotide on the template strand. For example, in some instances a terminator pairing with cytosine is present at about 3%, 5%, 10%, 15%, 20%, 25%, or 50% higher concentration than the average terminator concentration. In some instances a terminator pairing with thymine is present at about 3%, 5%, 10%, 15%, 20%, 25%, or 50% higher concentration than the average terminator concentration. In some instances a terminator pairing with guanine is present at about 3%, 5%, 10%, 15%, 20%, 25%, or 50% higher concentration than the average terminator concentration. In some instances a terminator pairing with adenine is present at about 3%, 5%, 10%, 15%, 20%, 25%, or 50% higher concentration than the average terminator concentration. In some instances a terminator pairing with uracil is present at about 3%, 5%, 10%, 15%, 20%, 25%, or 50% higher concentration than the average terminator concentration. Any nucleotide capable of terminating nucleic acid extension by a nucleic acid polymerase in some instances is used as a terminator nucleotide in the methods described herein. In some instances, a reversible terminator is used to terminate nucleic acid replication. In some instances, a non-reversible terminator is used to terminate nucleic acid replication. In some instances, non-limited examples of terminators include reversible and non-reversible nucleic acids and nucleic acid analogs, such as, e.g., 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, or any combination thereof. In one embodiment, terminator nucleotides are dideoxynucleotides. Other nucleotide modifications that terminate nucleic acid replication and may be suitable for practicing the invention include, without limitation, any modifications of the r group of the 3′ carbon of the deoxyribose such as inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. In some instances, terminators are polynucleotides comprising 1, 2, 3, 4, or more bases in length. In some instances, terminators do not comprise a detectable moiety or tag (e.g., mass tag, fluorescent tag, dye, radioactive atom, or other detectable moiety). In some instances, terminators do not comprise a chemical moiety allowing for attachment of a detectable moiety or tag (e.g., “click” azide/alkyne, conjugate addition partner, or other chemical handle for attachment of a tag). In some instances, all terminator nucleotides comprise the same modification that reduces amplification to at region (e.g., the sugar moiety, base moiety, or phosphate moiety) of the nucleotide. In some instances, at least one terminator has a different modification that reduces amplification. In some instances, all terminators have a substantially similar fluorescent excitation or emission wavelengths. In some instances, terminators without modification to the phosphate group are used with polymerases that do not have exonuclease proofreading activity. Terminators, when used with polymerases which have 3′->5′ proofreading exonuclease activity (such as, e.g., phi29) that can remove the terminator nucleotide, are in some instances further modified to make them exonuclease-resistant. For example, dideoxynucleotides are modified with an alpha-thio group that creates a phosphorothioate linkage which makes these nucleotides resistant to the 3′->5′ proofreading exonuclease activity of nucleic acid polymerases. Such modifications in some instances reduce the exonuclease proofreading activity of polymerases by at least 99.5%, 99%, 98%, 95%, 90%, or at least 85%. Non-limiting examples of other terminator nucleotide modifications providing resistance to the 3′->5′ exonuclease activity include in some instances: nucleotides with modification to the alpha group, such as alpha-thio dideoxynucleotides creating a phosphorothioate bond, C3 spacer nucleotides, locked nucleic acids (LNA), inverted nucleic acids, 2′ Fluoro bases, 3′ phosphorylation, 2′-O-Methyl modifications (or other 2′-O-alkyl modification), propyne-modified bases (e.g., deoxycytosine, deoxyuridine), L-DNA nucleotides, L-RNA nucleotides, nucleotides with inverted linkages (e.g., 5′-5′ or 3′-3′), 5′ inverted bases (e.g., 5′ inverted 2′,3′-dideoxy dT), methylphosphonate backbones, and trans nucleic acids. In some instances, nucleotides with modification include base-modified nucleic acids comprising free 3′ OH groups (e.g., 2-nitrobenzyl alkylated HOMedU triphosphates, bases comprising modification with large chemical groups, such as solid supports or other large moiety). In some instances, a polymerase with strand displacement activity but without 3′->5′exonuclease proofreading activity is used with terminator nucleotides with or without modifications to make them exonuclease resistant. Such nucleic acid polymerases include, without limitation, Bst DNA polymerase, Bsu DNA polymerase, Deep Vent (exo-) DNA polymerase, Klenow Fragment (exo-) DNA polymerase, Therminator DNA polymerase, and Vent_{R} (exo-).

### Primers and Amplicon Libraries

Described herein are amplicon libraries resulting from amplification of at least one target nucleic acid molecule. Such libraries are in some instances generated using the methods described herein, such as those using terminators. Such methods comprise use of strand displacement polymerases or factors, terminator nucleotides (reversible or irreversible), or other features and embodiments described herein. In some instances, reversible terminators are capable of removal by an exonuclease (e.g., or polymerase having exonuclease activity). In some instances, irreversible terminators are not capable of substantial removal by an exonuclease (e.g., or polymerase having exonuclease activity). In some instances, amplicon libraries generated by use of terminators described herein are further amplified in a subsequent amplification reaction (e.g., PCR). In some instances, subsequent amplification reactions do not comprise terminators. In some instances, amplicon libraries comprise polynucleotides, wherein at least 50%, 60%, 70%, 80%, 90%, 95%, or at least 98% of the polynucleotides comprise at least one terminator nucleotide. In some instances, the amplicon library comprises the target nucleic acid molecule from which the amplicon library was derived. The amplicon library comprises a plurality of polynucleotides, wherein at least some of the polynucleotides are direct copies (e.g., replicated directly from a target nucleic acid molecule, such as genomic DNA, RNA, or other target nucleic acid). For example, at least 5%, 10%, 20%, 30%, 40%, 50%, 60%, 70%, 80%, 90%, 95% or more than 95% of the amplicon polynucleotides are direct copies of the at least one target nucleic acid molecule. In some instances, at least 5% of the amplicon polynucleotides are direct copies of the at least one target nucleic acid molecule. In some instances, at least 10% of the amplicon polynucleotides are direct copies of the at least one target nucleic acid molecule. In some instances, at least 15% of the amplicon polynucleotides are direct copies of the at least one target nucleic acid molecule. In some instances, at least 20% of the amplicon polynucleotides are direct copies of the at least one target nucleic acid molecule. In some instances, at least 50% of the amplicon polynucleotides are direct copies of the at least one target nucleic acid molecule. In some instances, 3%-5%, 3-10%, 5%-10%, 10%-20%, 20%-30%, 30%-40%, 5%-30%, 10%-50%, or 15%-75% of the amplicon polynucleotides are direct copies of the at least one target nucleic acid molecule. In some instances, at least some of the polynucleotides are direct copies of the target nucleic acid molecule, or daughter (a first copy of the target nucleic acid) progeny. For example, at least 5%, 10%, 20%, 30%, 40%, 50%, 60%, 70%, 80%, 90%, 95% or more than 95% of the amplicon polynucleotides are direct copies of the at least one target nucleic acid molecule or daughter progeny. In some instances, at least 5% of the amplicon polynucleotides are direct copies of the at least one target nucleic acid molecule or daughter progeny. In some instances, at least 10% of the amplicon polynucleotides are direct copies of the at least one target nucleic acid molecule or daughter progeny. In some instances, at least 20% of the amplicon polynucleotides are direct copies of the at least one target nucleic acid molecule or daughter progeny. In some instances, at least 30% of the amplicon polynucleotides are direct copies of the at least one target nucleic acid molecule or daughter progeny. In some instances, 3%-5%, 3%-10%, 5%-10%, 10%-20%, 20%-30%, 30%-40%, 5%-30%, 10%-50%, or 15%-75% of the amplicon polynucleotides are direct copies of the at least one target nucleic acid molecule or daughter progeny. In some instances, direct copies of the target nucleic acid are 50-2500, 75-2000, 50-2000, 25-1000, 50-1000, 500-2000, or 50-2000 bases in length. In some instances, daughter progeny are 1000-5000, 2000-5000, 1000-10,000, 2000-5000, 1500-5000, 3000-7000, or 2000-7000 bases in length. In some instances, the average length of PTA amplification products is 25-3000 nucleotides in length, 50-2500, 75-2000, 50-2000, 25-1000, 50-1000, 500-2000, or 50-2000 bases in length. In some instance, amplicons generated from PTA are no more than 5000, 4000, 3000, 2000, 1700, 1500, 1200, 1000, 700, 500, or no more than 300 bases in length. In some instance, amplicons generated from PTA are 1000-5000, 1000-3000, 200-2000, 200-4000, 500-2000, 750-2500, or 1000-2000 bases in length. Amplicon libraries generated using the methods described herein in some instances comprise at least 1000, 2000, 5000, 10,000, 100,000, 200,000, 500,000 or more than 500,000 amplicons comprising unique sequences. In some instances, the library comprises at least 100, 200, 300, 400, 500, 600, 700, 800, 900, 1000, 1100, 1200, 1300, 1400, 1500, 2000, 2500, 3000, or at least 3500 amplicons. In some instances, at least 5%, 10%, 15%, 20%, 25%, 30% or more than 30% of amplicon polynucleotides having a length of less than 1000 bases are direct copies of the at least one target nucleic acid molecule. In some instances, at least 5%, 10%, 15%, 20%, 25%, 30% or more than 30% of amplicon polynucleotides having a length of no more than 2000 bases are direct copies of the at least one target nucleic acid molecule. In some instances, at least 5%, 10%, 15%, 20%, 25%, 30% or more than 30% of amplicon polynucleotides having a length of 3000-5000 bases are direct copies of the at least one target nucleic acid molecule. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are no more than 700-1200 bases in length. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are 700-1200 bases in length, and the daughter amplicons are 2500-6000 bases in length. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule or daughter amplicons. The number of direct copies may be controlled in some instances by the number of amplification cycles. In some instances, no more than 30, 25, 20, 15, 13, 11, 10, 9, 8, 7, 6, 5, 4, or 3 cycles are used to generate copies of the target nucleic acid molecule. In some instances, about 30, 25, 20, 15, 13, 11, 10, 9, 8, 7, 6, 5, 4, or about 3 cycles are used to generate copies of the target nucleic acid molecule. In some instances, 3, 4, 5, 6, 7, or 8 cycles are used to generate copies of the target nucleic acid molecule. In some instances, 2-4, 2-5, 2-7, 2-8, 2-10, 2-15, 3-5, 3-10, 3-15, 4-10, 4-15, 5-10 or 5-15 cycles are used to generate copies of the target nucleic acid molecule. Amplicon libraries generated using the methods described herein are in some instances subjected to additional steps, such as adapter ligation and further amplification. In some instances, such additional steps precede a sequencing step. In some instances, the cycles are PCR cycles. In some instances, the cycles represent annealing, extension, and denaturation. In some instances, the cycles represent annealing, extension, and denaturation which occur under isothermal or essentially isothermal conditions.

Methods described herein may additionally comprise one or more enrichment or purification steps. In some instances, one or more polynucleotides (such as cDNA, PTA amplicons, or other polynucleotide) are enriched during a method described herein. In some instances, polynucleotide probes are used to capture one or more polynucleotides. In some instances, probes are configured to capture one or more genomic exons. In some instances, a library of probes comprises at least 1000, 2000, 5000, 10,000, 50,000, 100,000, 200,000, 500,000, or more than 1 million different sequences. In some instances, a library of probes comprises sequences capable of binding to at least 10, 20, 50, 100, 200, 500, 1000, 2000, 5000, 10,000 or more than 10,000 genes. In some instances, probes comprise a moiety for capture by a solid support, such as biotin. In some instances, an enrichment step occurs after a PTA step. In some instances, an enrichment step occurs before a PTA step. In some instances, probes are configured to bind genomic DNA libraries. In some instances, probes are configured to bind cDNA libraries.

Amplicon libraries of polynucleotides generated from the PTA methods and compositions (terminators, polymerases, etc.) described herein in some instances have increased uniformity. Uniformity, in some instances, is described using a Lorenz curve, or other such method. Such increases in some instances lead to lower sequencing reads needed for the desired coverage of a target nucleic acid molecule (e.g., genomic DNA, RNA, or other target nucleic acid molecule). For example, no more than 50% of a cumulative fraction of polynucleotides comprises sequences of at least 80% of a cumulative fraction of sequences of the target nucleic acid molecule. In some instances, no more than 50% of a cumulative fraction of polynucleotides comprises sequences of at least 60% of a cumulative fraction of sequences of the target nucleic acid molecule. In some instances, no more than 50% of a cumulative fraction of polynucleotides comprises sequences of at least 70% of a cumulative fraction of sequences of the target nucleic acid molecule. In some instances, no more than 50% of a cumulative fraction of polynucleotides comprises sequences of at least 90% of a cumulative fraction of sequences of the target nucleic acid molecule. In some instances, uniformity is described using a Gini index (wherein an index of 0 represents perfect equality of the library and an index of 1 represents perfect inequality). In some instances, amplicon libraries described herein have a Gini index of no more than 0.55, 0.50, 0.45, 0.40, or 0.30. In some instances, amplicon libraries described herein have a Gini index of no more than 0.50. In some instances, amplicon libraries described herein have a Gini index of no more than 0.40. Such uniformity metrics in some instances are dependent on the number of reads obtained. For example, no more than 100 million, 200 million, 300 million, 400 million, or no more than 500 million reads are obtained. In some instances, the read length is about 50, 75, 100, 125, 150, 175, 200, 225, or about 250 bases in length. In some instances, uniformity metrics are dependent on the depth of coverage of a target nucleic acid. For example, the average depth of coverage is about 10×, 15×, 20×, 25×, or about 30×. In some instances, the average depth of coverage is 10-30×, 20-50×, 5-40×, 20-60×, 5-20×, or 10-20×. In some instances, amplicon libraries described herein have a Gini index of no more than 0.55, wherein about 300 million reads was obtained. In some instances, amplicon libraries described herein have a Gini index of no more than 0.50, wherein about 300 million reads was obtained. In some instances, amplicon libraries described herein have a Gini index of no more than 0.45, wherein about 300 million reads was obtained. In some instances, amplicon libraries described herein have a Gini index of no more than 0.55, wherein no more than 300 million reads was obtained. In some instances, amplicon libraries described herein have a Gini index of no more than 0.50, wherein no more than 300 million reads was obtained. In some instances, amplicon libraries described herein have a Gini index of no more than 0.45, wherein no more than 300 million reads was obtained. In some instances, amplicon libraries described herein have a Gini index of no more than 0.55, wherein the average depth of sequencing coverage is about 15×. In some instances, amplicon libraries described herein have a Gini index of no more than 0.50, wherein the average depth of sequencing coverage is about 15×. In some instances, amplicon libraries described herein have a Gini index of no more than 0.45, wherein the average depth of sequencing coverage is about 15×. In some instances, amplicon libraries described herein have a Gini index of no more than 0.55, wherein the average depth of sequencing coverage is at least 15×. In some instances, amplicon libraries described herein have a Gini index of no more than 0.50, wherein the average depth of sequencing coverage is at least 15×. In some instances, amplicon libraries described herein have a Gini index of no more than 0.45, wherein the average depth of sequencing coverage is at least 15×. In some instances, amplicon libraries described herein have a Gini index of no more than 0.55, wherein the average depth of sequencing coverage is no more than 15×. In some instances, amplicon libraries described herein have a Gini index of no more than 0.50, wherein the average depth of sequencing coverage is no more than 15×. In some instances, amplicon libraries described herein have a Gini index of no more than 0.45, wherein the average depth of sequencing coverage is no more than 15×. Uniform amplicon libraries generated using the methods described herein are in some instances subjected to additional steps, such as adapter ligation and further PCR amplification. In some instances, such additional steps precede a sequencing step.

Primers comprise nucleic acids used for priming the amplification reactions described herein. Such primers in some instances include, without limitation, random deoxynucleotides of any length with or without modifications to make them exonuclease resistant, random ribonucleotides of any length with or without modifications to make them exonuclease resistant, modified nucleic acids such as locked nucleic acids, DNA or RNA primers that are targeted to a specific genomic region, and reactions that are primed with enzymes such as primase. In the case of whole genome PTA, it is preferred that a set of primers having random or partially random nucleotide sequences be used. In a nucleic acid sample of significant complexity, specific nucleic acid sequences present in the sample need not be known and the primers need not be designed to be complementary to any particular sequence. Rather, the complexity of the nucleic acid sample results in a large number of different hybridization target sequences in the sample, which will be complementary to various primers of random or partially random sequence. The complementary portion of primers for use in PTA are in some instances fully randomized, comprise only a portion that is randomized, or be otherwise selectively randomized. The number of random base positions in the complementary portion of primers in some instances, for example, is from 20% to 100% of the total number of nucleotides in the complementary portion of the primers. In some instances, the number of random base positions in the complementary portion of primers is 10% to 90%, 15-95%, 20%-100%, 30%-100%, 50%-100%, 75-100% or 90-95% of the total number of nucleotides in the complementary portion of the primers. In some instances, the number of random base positions in the complementary portion of primers is at least 10%, 20%, 30%, 40%, 50%, 60%, 70%, 80%, or at least 90% of the total number of nucleotides in the complementary portion of the primers. Sets of primers having random or partially random sequences are in some instances synthesized using standard techniques by allowing the addition of any nucleotide at each position to be randomized. In some instances, sets of primers are composed of primers of similar length and/or hybridization characteristics. In some instances, the term “random primer” refers to a primer which can exhibit four-fold degeneracy at each position. In some instances, the term “random primer” refers to a primer which can exhibit three-fold degeneracy at each position. Random primers used in the methods described herein in some instances comprise a random sequence that is 3, 4, 5, 6, 7, 8, 10, 11, 12, 13, 14, 15, 16, 17, 18, 19, 20, or more bases in length. In some instances, primers comprise random sequences that are 3-20, 5-15, 5-20, 6-12, or 4-10 bases in length. Primers may also comprise non-extendable elements that limit subsequent amplification of amplicons generated thereof. For example, primers with non-extendable elements in some instances comprise terminators. In some instances, primers comprise terminator nucleotides, such as 1, 2, 3, 4, 5, 10, or more than 10 terminator nucleotides. Primers need not be limited to components which are added externally to an amplification reaction. In some instances, primers are generated in-situ through the addition of nucleotides and proteins which promote priming. For example, primase-like enzymes in combination with nucleotides is in some instances used to generate random primers for the methods described herein. Primase-like enzymes in some instances are members of the DnaG or AEP enzyme superfamily. In some instances, a primase-like enzyme is TthPrimPol. In some instances, a primase-like enzyme is T7 gp4 helicase-primase. Such primases are in some instances used with the polymerases or strand displacement factors described herein. In some instances, primases initiate priming with deoxyribonucleotides. In some instances, primases initiate priming with ribonucleotides. In some instances, primers are irreversible primers. In some instances, irreversible primers comprise phosphonothioate linkages.

The PTA amplification can be followed by selection for a specific subset of amplicons. Such selections are in some instances dependent on size, affinity, activity, hybridization to probes, or other known selection factor in the art. In some instances, selections precede or follow additional steps described herein, such as adapter ligation and/or library amplification. In some instances, selections are based on size (length) of the amplicons. In some instances, smaller amplicons are selected that are less likely to have undergone exponential amplification, which enriches for products that were derived from the primary template while further converting the amplification from an exponential into a quasi-linear amplification process. In some instances, amplicons comprising 50-2000, 25-5000, 40-3000, 50-1000, 200-1000, 300-1000, 400-1000, 400-600, 600-2000, or 800-1000 bases in length are selected. Size selection in some instances occurs with the use of protocols, e.g., utilizing solid-phase reversible immobilization (SPRI) on carboxylated paramagnetic beads to enrich for nucleic acid fragments of specific sizes, or other protocol known by those skilled in the art. Optionally or in combination, selection occurs through preferential ligation and amplification of smaller fragments during PCR while preparing sequencing libraries, as well as a result of the preferential formation of clusters from smaller sequencing library fragments during sequencing (e.g., sequencing by synthesis, nanopore sequencing, or other sequencing method) . . . . Other strategies to select for smaller fragments are also consistent with the methods described herein and include, without limitation, isolating nucleic acid fragments of specific sizes after gel electrophoresis, the use of silica columns that bind nucleic acid fragments of specific sizes, and the use of other PCR strategies that more strongly enrich for smaller fragments. Any number of library preparation protocols may be used with the PTA methods described herein. Amplicons generated by PTA are in some instances ligated to adapters (optionally with removal of terminator nucleotides). In some instances, amplicons generated by PTA comprise regions of homology generated from transposase-based fragmentation which are used as priming sites. In some instances, libraries are prepared by fragmenting nucleic acids mechanically or enzymatically. In some instances, libraries are prepared using tagmentation via transposomes. In some instances, libraries are prepared via ligation of adapters, such as Y-adapters, universal adapters, or circular adapters.

The non-complementary portion of a primer used in PTA can include sequences which can be used to further manipulate and/or analyze amplified sequences. An example of such a sequence is a “detection tag”. Detection tags have sequences complementary to detection probes and are detected using their cognate detection probes. There may be one, two, three, four, or more than four detection tags on a primer. There is no fundamental limit to the number of detection tags that can be present on a primer except the size of the primer. In some instances, there is a single detection tag on a primer. In some instances, there are two detection tags on a primer. When there are multiple detection tags, they may have the same sequence or they may have different sequences, with each different sequence complementary to a different detection probe. In some instances, multiple detection tags have the same sequence. In some instances, multiple detection tags have a different sequence.

Another example of a sequence that can be included in the non-complementary portion of a primer is an “address tag” that can encode other details of the amplicons, such as the location in a tissue section. In some instances, a cell barcode comprises an address tag. An address tag has a sequence complementary to an address probe. Address tags become incorporated at the ends of amplified strands. If present, there may be one, or more than one, address tag on a primer. There is no fundamental limit to the number of address tags that can be present on a primer except the size of the primer. When there are multiple address tags, they may have the same sequence or they may have different sequences, with each different sequence complementary to a different address probe. The address tag portion can be any length that supports specific and stable hybridization between the address tag and the address probe. In some instances, nucleic acids from more than one source can incorporate a variable tag sequence. This tag sequence can be up to 100 nucleotides in length, preferably 1 to 10 nucleotides in length, most preferably 4, 5 or 6 nucleotides in length and comprises combinations of nucleotides. In some instances, a tag sequence is 1-20, 2-15, 3-13, 4-12, 5-12, or 1-10 nucleotides in length For example, if six base-pairs are chosen to form the tag and a permutation of four different nucleotides is used, then a total of 4096 nucleic acid anchors (e.g. hairpins), each with a unique 6 base tag can be made. In some instances, tags identify the source of a sample or analyte. In some instances, tags uniquely identify every molecule in a population.

Primers described herein may be present in solution or immobilized on a solid support. In some instances, primers bearing sample barcodes and/or UMI sequences can be immobilized on a solid support. The solid support can be, for example, one or more beads. In some instances, individual cells are contacted with one or more beads having a unique set of sample barcodes and/or UMI sequences in order to identify the individual cell. In some instances, lysates from individual cells are contacted with one or more beads having a unique set of sample barcodes and/or UMI sequences in order to identify the individual cell lysates. In some instances, extracted nucleic acid from individual cells are contacted with one or more beads having a unique set of sample barcodes and/or UMI sequences in order to identify the extracted nucleic acid from the individual cell. The beads can be manipulated in any suitable manner as is known in the art, for example, using droplet actuators as described herein. The beads may be any suitable size, including for example, microbeads, microparticles, nanobeads and nanoparticles. In some embodiments, beads are magnetically responsive; in other embodiments beads are not significantly magnetically responsive. Non-limiting examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® available from Invitrogen Group, Carlsbad, CA), fluorescent microparticles and nanoparticles, coated magnetic microparticles and nanoparticles, ferromagnetic microparticles and nanoparticles, coated ferromagnetic microparticles and nanoparticles, and those described in U.S. Pat. Appl. Pub. No. US20050260686, US20030132538, US20050118574, 20050277197, 20060159962. Beads may be pre-coupled with an antibody, protein or antigen, DNA/RNA probe or any other molecule with an affinity for a desired target. In some embodiments, primers bearing sample barcodes and/or UMI sequences can be in solution. In certain embodiments, a plurality of droplets can be presented, wherein each droplet in the plurality bears a sample barcode which is unique to a droplet and the UMI which is unique to a molecule such that the UMI are repeated many times within a collection of droplets. In some embodiments, individual cells are contacted with a droplet having a unique set of sample barcodes and/or UMI sequences in order to identify the individual cell. In some embodiments, lysates from individual cells are contacted with a droplet having a unique set of sample barcodes and/or UMI sequences in order to identify the individual cell lysates. In some embodiments, extracted nucleic acid from individual cells are contacted with a droplet having a unique set of sample barcodes and/or UMI sequences in order to identify the extracted nucleic acid from the individual cell.

PTA primers may comprise a sequence-specific or random primer, a cell barcode and/or a unique molecular identifier (UMI) (e.g., linear primer and or hairpin primer). In some instances, a primer comprises a sequence-specific primer. In some instances, a primer comprises a random primer. In some instances, a primer comprises a cell barcode. In some instances, a primer comprises a sample barcode. In some instances, a primer comprises a unique molecular identifier. In some instances, primers comprise two or more cell barcodes. Such barcodes in some instances identify a unique sample source, or unique workflow. Such barcodes or UMIs are in some instances 5, 6, 7, 8, 9, 10, 11, 12, 15, 20, 25, 30, or more than 30 bases in length. Primers in some instances comprise at least 1000, 10,000, 50,000, 100,000, 250,000, 500,000, 10⁶, 10⁷, 10⁸, 10⁹, or at least 1010 unique barcodes or UMIs. In some instances primers comprise at least 8, 16, 96, or 384 unique barcodes or UMIs. In some instances a standard adapter is then ligated onto the amplification products prior to sequencing; after sequencing, reads are first assigned to a specific cell based on the cell barcode. Suitable adapters that may be utilized with the PTA method include, e.g., xGen® Dual Index UMI adapters available from Integrated DNA Technologies (IDT). Reads from each cell is then grouped using the UMI, and reads with the same UMI may be collapsed into a consensus read. The use of a cell barcode allows all cells to be pooled prior to library preparation, as they can later be identified by the cell barcode. The use of the UMI to form a consensus read in some instances corrects for PCR bias, improving the copy number variation (CNV) detection. In addition, sequencing errors may be corrected by requiring that a fixed percentage of reads from the same molecule have the same base change detected at each position. This approach has been utilized to improve CNV detection and correct sequencing errors in bulk samples. In some instances, UMIs are used with the methods described herein, for example, U.S. Pat. No. 8,835,358 discloses the principle of digital counting after attaching a random amplifiable barcode. Schmitt. et al and Fan et al. disclose similar methods of correcting sequencing errors. In some instances, a library is generated for sequencing using primers. In some instances, the library comprises fragments of 200-700 bases, 100-1000, 300-800, 300-550, 300-700, or 200-800 bases in length. In some instances, the library comprises fragments of at least 50, 100, 150, 200, 300, 500, 600, 700, 800, or at least 1000 bases in length. In some instances, the library comprises fragments of about 50, 100, 150, 200, 300, 500, 600, 700, 800, or about 1000 bases in length.

The methods described herein may further comprise additional steps, including steps performed on the sample or template. Such samples or templates in some instances are subjected to one or more steps prior to PTA. In some instances, samples comprising cells are subjected to a pre-treatment step. For example, cells undergo lysis and proteolysis to increase chromatin accessibility using a combination of freeze-thawing, Triton X-100, Tween 20, and Proteinase K. Other lysis strategies are also suitable for practicing the methods described herein. Such strategies include, without limitation, lysis using other combinations of detergent and/or lysozyme and/or protease treatment and/or physical disruption of cells such as sonication and/or alkaline lysis and/or hypotonic lysis. In some instances, the primary template or target molecule(s) is subjected to a pre-treatment step. In some instances, the primary template (or target) is denatured using sodium hydroxide, followed by neutralization of the solution. Other denaturing strategies may also be suitable for practicing the methods described herein. Such strategies may include, without limitation, combinations of alkaline lysis with other basic solutions, increasing the temperature of the sample and/or altering the salt concentration in the sample, addition of additives such as solvents or oils, other modification, or any combination thereof. In some instances, additional steps include sorting, filtering, or isolating samples, templates, or amplicons by size. In some instances, cells are lysed with mechanical (e.g., high pressure homogenizer, bead milling) or non-mechanical (physical, chemical, or biological). In some instances, physical lysis methods comprise heating, osmotic shock, and/or cavitation. In some instances, chemical lysis comprises alkali and/or detergents. In some instances, biological lysis comprises use of enzymes. Combinations of lysis methods are also compatible with the methods described herein. Non-limited examples of lysis enzymes include recombinant lysozyme, serine proteases, and bacterial lysins. In some instances, lysis with enzymes comprises use of lysozyme, lysostaphin, zymolase, cellulose, protease or glycanase. For example, after amplification with the methods described herein, amplicon libraries are enriched for amplicons having a desired length. In some instances, amplicon libraries are enriched for amplicons having a length of 50-2000, 25-1000, 50-1000, 75-2000, 100-3000, 150-500, 75-250, 170-500, 100-500, or 75-2000 bases. In some instances, amplicon libraries are enriched for amplicons having a length no more than 75, 100, 150, 200, 500, 750, 1000, 2000, 5000, or no more than 10,000 bases. In some instances, amplicon libraries are enriched for amplicons having a length of at least 25, 50, 75, 100, 150, 200, 500, 750, 1000, or at least 2000 bases.

Methods and compositions described herein may comprise buffers or other formulations. Such buffers are in some instances used for PTA, RT, or other method described herein. Such buffers in some instances comprise surfactants/detergent or denaturing agents (Tween-20, DMSO, DMF, pegylated polymers comprising a hydrophobic group, or other surfactant), salts (potassium or sodium phosphate (monobasic or dibasic), sodium chloride, potassium chloride, TrisHCI, magnesium chloride or sulfate, Ammonium salts such as phosphate, nitrate, or sulfate, EDTA), reducing agents (DTT, THP, DTE, beta-mercaptoethanol, TCEP, or other reducing agent) or other components (glycerol, hydrophilic polymers such as PEG). In some instances, buffers are used in conjunction with components such as polymerases, strand displacement factors, terminators, or other reaction component described herein. In some instances, buffers are used in conjunction with components such as polymerases, strand displacement factors, terminators, or other reaction component described herein. Buffers may comprise one or more crowding agents. In some instances, crowding reagents include polymers. In some instances, crowding reagents comprise polymers such as polyols. In some instances, crowding reagents comprise polyethylene glycol polymers (PEG). In some instances, crowding reagents comprise polysaccharides. Without limitation, examples of crowding reagents include ficoll (e.g., ficoll PM 400, ficoll PM 70, or other molecular weight ficoll), PEG (e.g., PEG1000, PEG 2000, PEG4000, PEG6000, PEG8000, or other molecular weight PEG), dextran (dextran 6, dextran 10, dextran 40, dextran 70, dextran 6000, dextran 138k, or other molecular weight dextran).

The nucleic acid molecules amplified according to the methods described herein may be sequenced and analyzed using methods known to those of skill in the art. Non-limiting examples of the sequencing methods which in some instances are used include, e.g., sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (Int. Pat. Appl. Pub. No. WO2006/073504), multiplex sequencing (U.S. Pat. Appl. Pub. No. US2008/0269068; Porreca et al., 2007, Nat. Methods 4:931), polymerized colony (POLONY) sequencing (U.S. Pat. Nos. 6,432,360, 6,485,944 and 6,511,803, and Int. Pat. Appl. Pub. No.

WO2005/082098), nanogrid rolling circle sequencing (ROLONY) (U.S. Pat. No. 9,624,538), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout), high-throughput sequencing methods such as, e.g., methods using Roche 454, Illumina Solexa, AB-SOLID, Helicos, Polonator platforms and the like, and light-based sequencing technologies (Landegren et al. (1998) Genome Res. 8:769-76; Kwok (2000) Pharmacogenomics 1:95-100; and Shi (2001) Clin. Chem.47:164-172). In some instances, the amplified nucleic acid molecules are shotgun sequenced. Sequencing of the sequencing library is in some instances performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis (array/colony-based or nanoball based).

Sequencing libraries generated using the methods described herein (e.g., PTA or RNAseq) may be sequenced to obtain a desired number of sequencing reads. In some instances, libraries are generated from a single cell or sample comprising a single cell (alone or part of a multiomics workflow). In some instances, libraries are sequenced to obtain at least 0.1, 0.2, 0.4, 0.5, 0.7, 0.8, 0.9, 1, 1.1, 1.2, 1.5, 2, 5, or at least 10 million reads. In some instances, libraries are sequenced to obtain no more than 0.1, 0.2, 0.4, 0.5, 0.7, 0.8, 0.9, 1, 1.1, 1.2, 1.5, 2, 5, or no more than 10 million reads. In some instances, libraries are sequenced to obtain about 0.1, 0.2, 0.4, 0.5, 0.7, 0.8, 0.9, 1, 1.1, 1.2, 1.5, 2, 5, or about 10 million reads. In some instances, libraries are sequenced to obtain 0.1-10, 0.1-5, 0.1-1, 0.2-1, 0.3-1.5, 0.5-1, 1-5, or 0.5-5 million reads per sample. In some instances, the number of reads is dependent on the size of the genome. In some in instances samples comprising bacterial genomes are sequenced to obtain 0.5-1 million reads. In some instances, libraries are sequenced to obtain at least 2, 4, 10, 20, 50, 100, 200, 300, 500, 700, or at least 900 million reads. In some instances, libraries are sequenced to obtain no more than 2, 4, 10, 20, 50, 100, 200, 300, 500, 700, or no more than 900 million reads. In some instances, libraries are sequenced to obtain about 2, 4, 10, 20, 50, 100, 200, 300, 500, 700, or about 900 million reads. In some in instances samples comprising mammalian genomes are sequenced to obtain 500-600 million reads. In some instances, the type of sequencing library (cDNA libraries or genomic libraries) are identified during sequencing. In some instances, cDNA libraries and genomic libraries are identified during sequencing with unique barcodes.

The term “cycle” when used in reference to a polymerase-mediated amplification reaction is used herein to describe steps of dissociation of at least a portion of a double stranded nucleic acid (e.g., a template from an amplicon, or a double stranded template, denaturation). hybridization of at least a portion of a primer to a template (annealing), and extension of the primer to generate an amplicon. In some instances, the temperature remains constant during a cycle of amplification (e.g., an isothermal reaction). In some instances, the number of cycles is directly correlated with the number of amplicons produced. In some instances, the number of cycles for an isothermal reaction is controlled by the amount of time the reaction is allowed to proceed.

### High Throughput Methods and Applications

High throughput devices and methods described herein may be used for a number of applications. Described herein are methods of identifying mutations in cells with the methods of multiomic analysis PTA, such as single cells. Use of the PTA method in some instances results in improvements over known methods, for example, MDA. PTA in some instances has lower false positive and false negative variant calling rates than the MDA method. Genomes, such as NA12878 platinum genomes, are in some instances used to determine if the greater genome coverage and uniformity of PTA would result in lower false negative variant calling rate. Without being bound by theory, it may be determined that the lack of error propagation in PTA decreases the false positive variant call rate. The amplification balance between alleles with the two methods is in some cases estimated by comparing the allele frequencies of the heterozygous mutation calls at known positive loci. In some instances, amplicon libraries generated using PTA are further amplified by PCR. In some instances, PTA is used in a workflow with additional analysis methods, such as RNAseq, methylome analysis or other method described herein.

Cells analyzed using the methods described herein in some instances comprise tumor cells. For example, circulating tumor cells can be isolated from a fluid taken from patients, such as but not limited to, blood, bone marrow, urine, saliva, cerebrospinal fluid, pleural fluid, pericardial fluid, ascites, or aqueous humor. In some instances, the tumor cells are obtained using FNA. In some instances, the tumor cells comprise are from one or more locations of a tissue region of a subject. The cells are then subjected to the methods described herein (e.g. PTA) and sequencing to determine mutation burden and mutation combination in each cell. These data are in some instances used for the diagnosis of a specific disease or as tools to predict treatment response. Similarly, in some instances cells of unknown malignant potential in some instances are isolated from fluid taken from patients, such as but not limited to, blood, bone marrow, urine, saliva, cerebrospinal fluid, pleural fluid, pericardial fluid, ascites, aqueous humor, blastocoel fluid, or collection media surrounding cells in culture. In some instances, a sample is obtained from collection media surrounding embryonic cells. In some instances, the cells of unknown malignant potential are obtained using FNA. In some instances, the cells of unknown malignant potential are obtained from one or more locations of a tissue region of a subject. After utilizing the methods described herein and sequencing, such methods are further used to determine mutation burden and mutation combination in each cell. These data are in some instances used for the diagnosis of a specific disease or as tools to predict progression of a premalignant state to overt malignancy. In some instances, cells can be isolated from primary tumor samples. The cells can then undergo PTA and sequencing to determine mutation burden and mutation combination in each cell. These data can be used for the diagnosis of a specific disease or are as tools to predict the probability that a patient's malignancy is resistant to available anti-cancer drugs. By exposing samples to different chemotherapy agents, it has been found that the major and minor clones have differential sensitivity to specific drugs that does not necessarily correlate with the presence of a known “driver mutation,” suggesting that combinations of mutations within a clonal population determine its sensitivities to specific chemotherapy drugs. Without being bound by theory, these findings suggest that a malignancy may be easier to eradicate if premalignant lesions that have not yet expanded are and evolved into clones are detected whose increased number of genome modification may make them more likely to be resistant to treatment. See, Ma et al., 2018, “Pan-cancer genome and transcriptome analyses of 1,699 pediatric leukemias and solid tumors.” A single-cell genomics protocol is in some instances used to detect the combinations of somatic genetic variants in a single cancer cell, or clonotype, within a mixture of normal and malignant cells that are isolated from patient samples. This technology is in some instances further utilized to identify clonotypes that undergo positive selection after exposure to drugs, both in vitro and/or in patients. By comparing the surviving clones exposed to chemotherapy compared to the clones identified at diagnosis, a catalog of cancer clonotypes can be created that documents their resistance to specific drugs. PTA methods in some instances detect the sensitivity of specific clones in a sample composed of multiple clonotypes to existing or novel drugs, as well as combinations thereof, where the method can detect the sensitivity of specific clones to the drug. This approach in some instances shows efficacy of a drug for a specific clone that may not be detected with current drug sensitivity measurements that consider the sensitivity of all cancer clones together in one measurement. When the PTA described herein are applied to patient samples collected at the time of diagnosis in order to detect the cancer clonotypes in a given patient's cancer, a catalog of drug sensitivities may then be used to look up those clones and thereby inform oncologists as to which drug or combination of drugs will not work and which drug or combination of drugs is most likely to be efficacious against that patient's cancer. The PTA may be used for analysis of samples comprising groups of cells. In some instances, a sample comprises neurons or glial cells. In some instances, the sample comprises nuclei.

Described herein are methods of measuring the gene expression or mutation alteration in combination with the mutagenicity of an environmental factor. For example, cells (single or a population) are exposed to a potential environmental condition. For example, cells such originating from organs (liver, pancreas, lung, colon, thyroid, or other organ), tissues (skin, or other tissue), blood, or other biological source are in some instances used with the method. In some instances, an environmental condition comprises heat, light (e.g. ultraviolet), radiation, a chemical substance, or any combination thereof. After an amount of exposure to the environmental condition, in some instances minutes, hours, days, or longer, single cells are isolated and subjected to the PTA method. In some instances, molecular barcodes and unique molecular identifiers are used to tag the sample. The sample is sequenced and then analyzed to identify gene expression alterations and or resulting from mutations resulting from exposure to the environmental condition. In some instances, such mutations are compared with a control environmental condition, such as a known non-mutagenic substance, vehicle/solvent, or lack of an environmental condition. Such analysis in some instances not only provides the total number of mutations caused by the environmental condition, but also the locations and nature of such mutations. Patterns are in some instances identified from the data, and may be used for diagnosis of diseases or conditions. In some instances, patterns are used to predict future disease states or conditions. In some instances, the methods described herein measure the mutation burden, locations, and patterns in a cell after exposure to an environmental agent, such as, e.g., a potential mutagen or teratogen. This approach in some instances is used to evaluate the safety of a given agent, including its potential to induce mutations that can contribute to the development of a disease. For example, the method could be used to predict the carcinogenicity or teratogenicity of an agent to specific cell types after exposure to a specific concentration of the specific agent.

Described herein are methods of identifying gene expression alteration in combination with the mutations in animal, plant or microbial cells that have undergone genome editing (e.g., using CRISPR technologies). Such cells in some instances can be isolated and subjected to PTA and sequencing to determine mutation burden and mutation combination in each cell. The per-cell mutation rate and locations of mutations that result from a genome editing protocol are in some instances used to assess the safety of a given genome editing method.

Described herein are methods of determining gene expression alteration in combination with the mutations in cells that are used for cellular therapy, such as but not limited to the transplantation of induced pluripotent stem cells, transplantation of hematopoietic or other cells that have not be manipulated, or transplantation of hematopoietic or other cells that have undergone genome edits. The cells can then undergo PTA and sequencing to determine mutation burden and mutation combination in each cell. The per-cell mutation rate and locations of mutations in the cellular therapy product can be used to assess the safety and potential efficacy of the product.

Cells for use with the PTA method may be fetal cells, such as embryonic cells. In some embodiments, PTA is used in conjunction with non-invasive preimplantation genetic testing (NIPGT). In a further embodiment, cells can be isolated from blastomeres that are created by in vitro fertilization. The cells can then undergo PTA and sequencing to determine the burden and combination of potentially disease predisposing genetic variants in each cell. The gene expression alteration in combination with the mutation profile of the cell can then be used to extrapolate the genetic predisposition of the blastomere to specific diseases prior to implantation. In some instances embryos in culture shed nucleic acids that are used to assess the health of the embryo using low pass genome sequencing. In some instances, embryos are frozen-thawed. In some instances, nucleic acids are obtained from blastocyte culture conditioned medium (BCCM), blastocoel fluid (BF), or a combination thereof. In some instances, PTA analysis of fetal cells is used to detect chromosomal abnormalities, such as fetal aneuploidy. In some instances, PTA is used to detect diseases such as Down's or Patau syndromes. In some instances, frozen blastocytes are thawed and cultured for a period of time before obtaining nucleic acids for analysis (e.g., culture media, BF, or a cell biopsy). In some instances, blastocytes are cultured for no more than 4, 6, 8, 12, 16, 24, 36, 48, or no more than 64 hours prior to obtaining nucleic acids for analysis.

In another embodiment, microbial cells (e.g., bacteria, fungi, protozoa) can be isolated from plants or animals (e.g., from microbiota samples [e.g., GI microbiota, skin microbiota, etc.] or from bodily fluids such as, e.g., blood, bone marrow, urine, saliva, cerebrospinal fluid, pleural fluid, pericardial fluid, ascites, or aqueous humor). In addition, microbial cells may be isolated from indwelling medical devices, such as but not limited to, intravenous catheters, urethral catheters, cerebrospinal shunts, prosthetic valves, artificial joints, or endotracheal tubes. The cells can then undergo PTA and sequencing to determine the identity of a specific microbe, as well as to detect the presence of microbial genetic variants that predict response (or resistance) to specific antimicrobial agents. These data can be used for the diagnosis of a specific infectious disease and/or as tools to predict treatment response.

Described herein are methods generating amplicon libraries from samples comprising short nucleic acid using the PTA methods described herein. In some instances, PTA leads to improved fidelity and uniformity of amplification of shorter nucleic acids. In some instances, nucleic acids are no more than 2000 bases in length. In some instances, nucleic acids are no more than 1000 bases in length. In some instances, nucleic acids are no more than 500 bases in length. In some instances, nucleic acids are no more than 200, 400, 750, 1000, 2000 or 5000 bases in length. In some instances, samples comprising short nucleic acid fragments include but at not limited to ancient DNA (hundreds, thousands, millions, or even billions of years old), FFPE (Formalin-Fixed Paraffin-Embedded) samples, cell-free DNA, or other sample comprising short nucleic acids.

### EXAMPLES

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### Example 1: Design and Execution of a Multiomics Workflow

### Overview

Discovering genomic variation in the absence of information about transcriptional consequence of that variation or, conversely, a transcriptional signature without understanding underlying genomic contributions, hinders understanding of molecular mechanisms of disease. To assess this genomic and transcriptomic coordination, a multiomics method was developed to extract this information out of the individual cell. The workflow unifies template-switching full-transcript RNA-Seq chemistry and whole genome amplification (WGA), followed by affinity purification of first-strand cDNA and subsequent separation of the RNA/DNA fractions for sequencing library preparation. In the multiomics methodology the attributes of primary template-directed amplification (PTA) were leveraged to enable accurate assessment of single-nucleotide variation as a DNA feature—which is not achieved with other workflows to assess DNA+RNA information in the same cell.

A single-well integration of single-cell transcriptome and genome amplification where a standard PTA reaction was modified to include a reverse transcription (RT) step prior to single-cell genome amplification was designed and executed, and designated as multiomic enrichment. In this workflow, PTA amplifies the genomes of single cells immediately after the RT reaction is concluded in a single-well reaction. Using template switch-based reverse transcription, barcoded first-strand cDNA molecules were created that were affinity purified and pre-amplified prior to RNA-Seq sequencing library creation. The net result from the combined amplification reaction was a biotin labeled cDNA pool derived primarily from the cytosolic transcripts, available for streptavidin purification, and a pool of amplified genomic material from the single cell. At the conclusion of the genome amplification reaction the cDNA fraction is separated from the amplified genome material whereby libraries from each pool were created. The resulting sequencing data offered the ability to define both genomic and transcriptomic plasticity at single-cell resolution. Specifically, the delineation of isoform expression, combined with ability to annotate the underlying structural variation and single nucleotide changes from the genome of the same cell, allowed the assessment of genomic “penetrance”, and the definition of mechanisms that drive single-cell fate.

Prior multiomic efforts pioneered the pairing of genomic and transcriptomic information from the same single cell but have the primary shortcoming of incomplete genome coverage and associated non-uniformity of coverage—leaving uncovered genomic valleys that may harbor deleterious single nucleotide variants that would remain undetected. Indeed, multiple displacement amplification (MDA) drives the genomic amplification of G&T-seq and DR-Seq has genomic amplification uniformity comparable to that of MALBAC, both of which are outperformed by PTA in terms of genomic coverage, allelic balance and SNV calling metrics. In one example, definition of clonal evolution at the SNV/CNV level in a primary patient sample was accomplished utilizing G&T-seq, yet was limited to a candidate gene survey of exome-level data whereby clusters where defined by 59 oncogenes and another studying employing G&T-seq limited their analysis to the RNA workflow of the method to take advantage of the low input requirement, without assessment of genomic level data. Thus, addressed herein is an unmet need to add genome-wide, high sensitivity and high precision SNV calling capability to a joint DNA/RNA single-cell methodology. Further, the importance of these measurements is demonstrated, whereby single nucleotide variation fundamentally affects cell state and tumor progression.

Provided herein are the utility of these unified “-omic” layers, highlighting heterogenous genomic variation and consequential phenotypic alterations in single cells that both are correlated with the development of resistance to a targeted therapeutic in a cell line model of acute myeloid leukemia, and in oncogenic mechanisms in primary breast cancer cells whereby the insights gained could not be inferred by a single dataset (genome or transcriptome) alone.

### Amplification Product Yield of RNA+DNA Multiomics Workflow

Prior to demonstrating biological utility of the multiomics method described herein, in a cell line drug resistance model and in a primary patient sample, the technical performance of the methodology using a benchmark cell line 1000 Genomes cell line, NA12878 was examined. The RNA and DNA arms of the protocol were first assessed using metrics from the template-switching RNA-Seq chemistry or PTA chemistry in isolation to compare to the metrics when the chemistries were unified in the combined multiomics protocol.

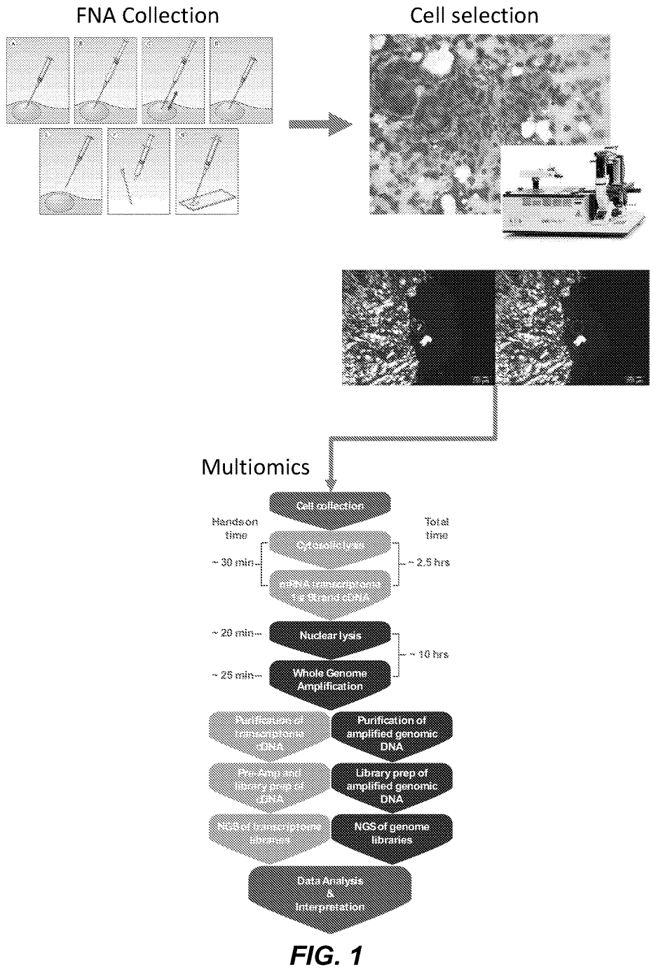

Multiomics data with FACS-sorted NA12878 single cells was generated with purified total NA12878 RNA or genomic DNA as amplification controls using the workflow shown in FIG. 1. Approximately 1-1.5 μg of DNA amplification product from single cell genomes and approximately 100-200 ng of cDNA product representing the single cell transcriptome was obtained. Importantly, no-template control (NTC) reactions showed lack of detectable product and additionally there was negligible (<50 ng) yield in the DNA fraction from control RNA input using Qubit fluorometer (ThermoFisher). Low-level background amplification of the genomic DNA control input in the cDNA fraction was observed, due to known promiscuity of reverse transcriptase in the absence of mRNA template. By contrast, this background amplification does not occur in reactions with single cells as the genome material is sequestered in the non-lysed nucleus during the reverse transcription workflow of multiomics.

### PTA Modifications

The PTA method was modified for use in a multiomics workflow. After reverse transcription has completed, dUTP was added to the normal nucleotide mix (dATP, dCTP, dGTP, dTTP) during phi29 amplification (red dot), resulting in PTA amplification products derived from the original single-cell or low-input template DNA being “marked” with dUTP. A UDG incubation step occurred on beads after affinity purification and washes of the cDNA, to digest the background dUTP-marked PTA product prior to preamplification of the cDNA (green dot). For library preparation, the cDNA libraries utilized a normal high-fidelity polymerase, however, the PTA-derived libraries representing the DNA arm of the multiomics workflow used a uracil tolerant polymerase in order to amplify the library ligation products of uracil-containing PTA product (yellow dot). The number of expressed genes detected was reduced following UDG treatment; indicating that transcript counts in the absence of UDG treatment were likely compounded by DNA (PTA) background. IGV visualization (700 kb region, harboring 3 genes) of intergenic read background removal upon UDG scheme. Each row was a single-cell (NA12878) Multiomic RNA fraction library. DNA background reads was seen in the top two control RNA libraries when PTA was performed lacking dUTP, and these background reads progressively diminished as more dUTP is included during PTA. The ratio of nucleotides was 1:1 dUTP:dTTP; PTA reactions containing dUTP exclusively with no dTTP were slower kinetically. The DNA background removal benefits of increased dUTP in the PTA reaction did not adversely affect allelic balance and SNV calling precision and sensitivity metrics.

Some polymerases stall or have reduced efficiency when amplifying templates comprising uracil. Uracil tolerant polymerases may be used with the methods described herein to amplify uracil-containing templates (e.g., with PTA). In some instances, a uracil tolerant polymerase maintains at least 50, 60, 70, 80, 85, 90, 95, 97, or 99% polymerase activity when amplifying a template comprising uracil as compared to a template without uracil. In some instances a uracil tolerant polymerase is derived from archaea, yeast, or bacterial species. In some instances a uracil tolerant polymerase comprises DNA polymerases ¿ and 8 from S. cerevisiae, and E. coli DNA polymerase III, PolA-type polymerases such as Taq, KAPA HiFi Uracil+DNA Polymerase (Kapa biosystems, Q5U), KOD Multi & Epi DNA Polymerase, FastStart Taq (Roche), Taq2000 (Agilent Technologies), FailSafe Enzyme (Epicentre) or Thermo PhusionU. In some instances, a uracil tolerant polymerase comprises a sequence having at least 70%, 75%, 80%, 85%, 90%, 95%, 97%, or 99% identity with DNA polymerases & and 8 from S. cerevisiae, and E. coli DNA polymerase III, PolA-type polymerases such as Taq, KAPA HiFi Uracil+DNA Polymerase (Kapa biosystems, Q5U), KOD Multi & Epi DNA Polymerase, FastStart Taq (Roche), Taq2000 (Agilent Technologies), FailSafe Enzyme (Epicentre) or Thermo PhusionU. In some instance a uracil tolerant polymerase comprises a modification to one or more amino acid residues in the dUTP binding pocket.

### Comparative Genomic Performance of Multiomics Workflow

As default practice prior to passing single cell samples to deep sequencing for SNV analysis low-pass QC sequencing was performed, and as part of the analysis pipeline, an estimation of library complexity with the PreSeq count algorithm determined. QC standards set for genomic DNA only (product solution for PTA) are >3.0E9 PreSeq count value upon low-pass sequencing, an empirically-defined proxy for genomic coverage and uniformity that predicts high-depth sequencing will yield strong allelic balance and high sensitivity and precision of single nucleotide variant calling. The average PreSeq count of single cells from Table 1A was 3.76E9 with a standard deviation of +/−2.27E8. The overall robust performance of single cells and genomic DNA controls warranted subsequent deep sequencing for metric comparison of classical PTA to PTA from the multiomic workflow.

TABLE 1A

| | PreSeq | % | % | % | % | Insert | Total |
|---|---|---|---|---|---|---|---|
| mple Name | Count | chrM | Chimeras | Aligned | Error | Size | Reads |
| MOLM13-DNA- | 3,378,959,196 | 1.41 | 10.55 | 99.89 | 0.81 | 209 | 533,283,408 |
| P_SC10 | | | | | | | |
| MOLM13-DNA- | 3,047,245,546 | 1.22 | 13.93 | 99.82 | 0.91 | 271 | 547,092,936 |
| P_SC11 | | | | | | | |
| MOLM13-DNA- | 3,478,446,000 | 0.86 | 12.35 | 99.85 | 0.84 | 244 | 480,104,446 |
| R_SC12 | | | | | | | |
| MOLM13-DNA- | 3,654,038,904 | 0.62 | 13.6 | 99.83 | 0.88 | 271 | 385,229,788 |
| R_SC13 | | | | | | | |
| MOLM13-DNA- | 3,254,232,372 | 1.32 | 13.77 | 99.84 | 0.88 | 271 | 573,459,994 |
| R_SC14 | | | | | | | |
| MOLM13-DNA- | 3,542,833,429 | 1.27 | 13.51 | 99.84 | 0.88 | 270 | 507,961,822 |
| R_SC15 | | | | | | | |
| MOLM13-DNA- | 3,632,455,433 | 1.31 | 13.53 | 99.84 | 0.87 | 268 | 447,380,990 |
| R_SC16 | | | | | | | |
| MOLM13-DNA- | 3,654,615,714 | 0.74 | 12.93 | 99.84 | 0.87 | 268 | 377,431,838 |
| R_SC17 | | | | | | | |
| MOLM13-DNA- | 3,367,781,398 | 1.33 | 14 | 99.84 | 0.86 | 271 | 487,173,864 |
| R_SC18 | | | | | | | |
| MOLM13-DNA- | 3,727,151,763 | 1.24 | 14.07 | 99.82 | 0.91 | 271 | 362,465,154 |
| R_SC19 | | | | | | | |
| MOLM13-DNA- | 3,679,483,372 | 0.84 | 12.59 | 99.81 | 0.88 | 270 | 509,963,664 |
| P_SC20 | | | | | | | |
| MOLM13-DNA- | 3,756,601,103 | 1.12 | 13.31 | 99.79 | 0.94 | 271 | 426,871,548 |
| R_SC21 | | | | | | | |
| MOLM13-DNA- | 3,643,238,259 | 0.95 | 12.69 | 99.82 | 0.87 | 266 | 510,327,482 |
| R_SC22 | | | | | | | |
| MOLM13-DNA- | 3,654,228,480 | 0.98 | 12.39 | 99.76 | 0.92 | 271 | 472,760,368 |
| P_SC23 | | | | | | | |
| MOLM13-DNA- | 3,716,521,996 | 0.74 | 12.16 | 99.81 | 0.87 | 271 | 448,199,656 |
| P_SC24 | | | | | | | |
| MOLM13-DNA- | 3,721,188,640 | 0.81 | 12.25 | 99.8 | 0.88 | 271 | 475,216,948 |
| P_SC25 | | | | | | | |
| MOLM13-DNA- | 3,561,816,152 | 0.91 | 12.36 | 99.8 | 0.87 | 260 | 373,985,162 |
| P_SC26 | | | | | | | |
| MOLM13-DNA- | 3,612,063,548 | 1.21 | 12.62 | 99.8 | 0.91 | 270 | 481,944,292 |
| P_SC27 | | | | | | | |
| MOLM13-DNA- | 3,663,289,670 | 0.73 | 11.79 | 99.8 | 0.87 | 237 | 439,568,964 |
| P_SC28 | | | | | | | |
| MOLM13-DNA- | 3,689,642,274 | 1.83 | 12.84 | 99.85 | 0.87 | 271 | 444,353,302 |
| P_SC29 | | | | | | | |
| DCIS1a-DNA- | 3,798,856,998 | 1.27 | 11.65 | 99.84 | 0.85 | 234 | 396,346,406 |
| EpCAM-H_SC10 | | | | | | | |
| DCIS1a-DNA- | 3,903,824,812 | 1.27 | 11.96 | 99.85 | 0.84 | 271 | 361,861,448 |
| EpCAM-H_SC11 | | | | | | | |
| DCIS1a-DNA- | 3,853,848,682 | 0.93 | 11.98 | 99.83 | 0.87 | 268 | 344,868,038 |
| EpCAM-H_SC16 | | | | | | | |
| DCIS1a-DNA- | 3,826,217,668 | 0.76 | 11.9 | 99.84 | 0.84 | 271 | 587,467,028 |
| EpCAM-H_SC3 | | | | | | | |
| DCIS1a-DNA- | 3,860,673,146 | 0.6 | 10.86 | 99.84 | 0.86 | 267 | 491,703,926 |
| EpCAM-H_SC4 | | | | | | | |
| DCIS1a-DNA- | 3,823,230,915 | 0.65 | 11.37 | 99.85 | 0.85 | 271 | 517,419,546 |
| EpCAM-H_SC5 | | | | | | | |
| DCIS1a-DNA- | 4,041,611,753 | 0.77 | 11.04 | 99.84 | 0.87 | 271 | 457,263,284 |
| EpCAM-H_SC6 | | | | | | | |
| DCIS1a-DNA- | 3,872,528,492 | 0.38 | 11.02 | 99.83 | 0.87 | 266 | 425,639,760 |
| EpCAM-H_SC7 | | | | | | | |
| DCIS1a-DNA- | 3,742,075,389 | 0.46 | 10.45 | 99.87 | 0.83 | 242 | 497,436,780 |
| EpCAM-H_SC8 | | | | | | | |
| DCIS1a-DNA- | 2,669,576,486 | 1.41 | 11.64 | 99.84 | 0.86 | 248 | 472,307,222 |
| EpCAM-H_SC9 | | | | | | | |
| DCIS1a-DNA- | 3,883,582,122 | 0.26 | 11.14 | 99.84 | 0.85 | 237 | 526,130,674 |
| EpCAM-L_SC3 | | | | | | | |
| DCIS1a-DNA- | 3,903,158,379 | 0.18 | 11.71 | 99.87 | 0.82 | 231 | 405,982,650 |
| EpCAM-L_SC8 | | | | | | | |
| NA12878a-DNA_SC10 | 3,804,223,358 | 0.5 | 11.17 | 99.83 | 0.86 | 270 | 341,176,288 |
| NA12878a-DNA_SC11 | 3,879,433,072 | 0.54 | 11.18 | 99.85 | 0.84 | 271 | 391,778,760 |
| NA12878a-DNA_SC12 | 3,851,844,096 | 0.42 | 11.54 | 99.84 | 0.85 | 271 | 381,523,468 |
| NA12878a-DNA_SC13 | 3,896,350,430 | 0.53 | 10.55 | 99.83 | 0.86 | 270 | 488,312,070 |
| NA12878a-DNA_SC16 | 3,754,881,507 | 0.63 | 11.12 | 99.83 | 0.87 | 270 | 476,517,794 |
| NA12878a-DNA_SC2 | 3,696,174,079 | 0.6 | 11.5 | 99.8 | 0.88 | 270 | 404,730,258 |
| NA12878a-DNA_SC3 | 3,600,119,598 | 0.4 | 11.24 | 99.81 | 0.89 | 271 | 384,398,982 |
| NA12878a-DNA_SC4 | 3,838,000,444 | 0.86 | 11.52 | 99.84 | 0.84 | 271 | 459,405,210 |
| NA12878a-DNA_SC5 | 3,855,303,158 | 0.51 | 11.46 | 99.85 | 0.83 | 271 | 464,258,980 |
| NA12878a-DNA_SC9 | 3,653,170,378 | 0.54 | 11.02 | 99.86 | 0.83 | 265 | 454,950,012 |
| DCIS1b-EpCAM- | 3,858,356,596 | 0.43 | 12.6 | 99.86 | 0.82 | 270 | 1,002,712,220 |
| H_SC1 | | | | | | | |
| DCIS1b-EpCAM- | 3,821,814,480 | 0.77 | 11.54 | 99.85 | 0.81 | 233 | 795,376,626 |
| H_SC10 | | | | | | | |
| DCIS1b-EpCAM- | 3,793,373,465 | 0.86 | 11.93 | 99.86 | 0.8 | 265 | 919,124,034 |
| H_SC11 | | | | | | | |
| DCIS1b-EpCAM- | 3,907,483,851 | 0.32 | 11.51 | 99.85 | 0.81 | 240 | 878,459,470 |
| H_SC12 | | | | | | | |
| DCIS1b-EpCAM- | 3,822,942,159 | 0.62 | 11.63 | 99.84 | 0.82 | 237 | 1,034,254,730 |
| H_SC13 | | | | | | | |
| DCIS1b-EpCAM- | 3,825,779,695 | 0.57 | 11.6 | 99.82 | 0.9 | 271 | 204,956,332 |
| H_SC15 | | | | | | | |
| DCIS1b-EpCAM- | 3,781,245,576 | 0.95 | 10.43 | 99.86 | 0.81 | 216 | 1,125,854,978 |
| H_SC16 | | | | | | | |
| DCIS1b-EpCAM- | 3,834,539,384 | 1.01 | 12.68 | 99.84 | 0.82 | 271 | 865,697,460 |
| H_SC4 | | | | | | | |
| DCIS1b-EpCAM- | 3,916,117,911 | 1.07 | 12.47 | 99.86 | 0.83 | 270 | 1,008,802,612 |
| H_SC5 | | | | | | | |
| DCIS1b-EpCAM- | 3,815,192,273 | 1.21 | 11.6 | 99.79 | 0.91 | 239 | 263,486,188 |
| H_SC6 | | | | | | | |
| DCIS1b-EpCAM- | 3,894,130,144 | 1.13 | 11.67 | 99.81 | 0.93 | 271 | 237,920,488 |
| H_SC7 | | | | | | | |
| DCIS1b-EpCAM- | 3,906,070,366 | 0.84 | 12.86 | 99.88 | 0.82 | 270 | 803,195,790 |
| H_SC9 | | | | | | | |
| NA12878b-DNA_SC1 | 3,957,877,510 | 0.34 | 11.55 | 99.8 | 0.86 | 270 | 476,648,274 |
| NA12878b-DNA_SC10 | 3,930,104,125 | 0.34 | 11.58 | 99.85 | 0.83 | 238 | 416,467,862 |
| NA12878b-DNA_SC11 | 3,979,659,349 | 0.44 | 11.68 | 99.82 | 0.85 | 267 | 443,612,012 |
| NA12878b-DNA_SC12 | 3,970,854,286 | 0.65 | 11.69 | 99.84 | 0.85 | 238 | 473,077,904 |
| NA12878b-DNA_SC15 | 3,957,961,760 | 0.69 | 11.53 | 99.85 | 0.82 | 239 | 607,659,734 |
| NA12878b-DNA_SC2 | 3,965,896,761 | 0.53 | 11.55 | 99.82 | 0.84 | 243 | 544,683,908 |
| NA12878b-DNA_SC3 | 3,910,878,320 | 0.2 | 11.35 | 99.82 | 0.85 | 271 | 435,263,252 |
| NA12878b-DNA_SC4 | 3,927,002,572 | 0.68 | 11.78 | 99.83 | 0.83 | 268 | 428,068,212 |
| NA12878b-DNA_SC7 | 3,944,483,107 | 0.49 | 11.32 | 99.83 | 0.83 | 270 | 325,712,176 |
| NA12878b-DNA_SC9 | 4,027,651,804 | 0.4 | 11.92 | 99.83 | 0.85 | 270 | 250,082,914 |
| DCIS1c-EpCAM- | 3,818,963,492 | 0.23 | 11.68 | 99.85 | 0.81 | 271 | 1,082,729,250 |
| L_SC2 | | | | | | | |
| DCIS1c-EpCAM- | 3,909,620,601 | 0.21 | 11.87 | 99.83 | 0.84 | 266 | 1,319,911,956 |
| L_SC8 | | | | | | | |

Upon high-depth sequencing (2×150 bp, down-sampling to 4.5E8 total reads, ~20× genome depth) and processing through our pipeline, allelic balance was reviewed, (ability to represent both alleles through enrichment and a strength of genomic PTA methodology). The inverse of allelic drop out (ADO) is allelic balance, which is the proportion of known heterozygous loci that are called heterozygous following sequencing. Variants within these loci have allele frequencies between 10% and 90% at each locus. A review of allelic balance of the multiomics workflow showed 85.5% (+/−3.4%), which was closely comparable to the 88.2% (+/−4%) for genomic DNA only workflow, across 10 replicates each. Genomic coverage at a range of depths did not significantly differ between the workflows. Lastly, it was critical to demonstrate that the allelic balance and coverage obtained from the multiomics workflow culminated in the ability to call SNVs with confidence. Individual multiomics of NA12878 cells with a SNV calling sensitivity range of 0.90-0.95 and with precision >0.99, akin to genomic DNA-only data was obtained. Collectively, these data suggested that, despite the upstream reverse transcription chemistry modifications to generate transcriptome data, amplification performance of single-cell genomes by PTA persists in performance.

### Comparative Transcriptomic Performance of Multiomics Workflow

In choosing a transcriptomic scheme to unite with PTA one goal was to be as comprehensive as possible in capturing the diversity of RNA-based modes of oncogenic and drug resistance mechanisms, and, equally as importantly, to enable the ascertainment of genomic lesions manifesting at the RNA level. A template-switching reverse transcription scheme was designed for the multiomics workflow that captured full-transcript information as opposed to either 5′ or 3′ end counting to enhance ability to detect isoforms and identify fusions. This chemistry enables even coverage across transcripts, where increased coverage of the 5′ region (top) which typically is affected by degradation (or reverse transcriptase performance) proportional to the distance from 3′-polyA, is shown. This confirms behavior of the template-switching chemistry in the RNA arm workflow. The distribution of read depth across gene bodies of a set of housekeeping genes was determined, with all exons equally represented. Feature quantification across the defined transcriptome was obtained, highlighting the ability to identify a variety of transcript bodies. Most notably, increased 5′ coding and intronic regions in the multiomics chemistry was observed overall, with intergenic background routinely below 5% of aligned reads, providing a broader space for isoform detection.

As further performance benchmarking of cell quality post mapping to reference transcriptome, performance patterns were established of common metrics with well characterized Human Brain Reference RNA (HBRR) and Universal Human Reference RNA (UHRR) as additions to the NA12878 cell line and displayed composite features. Read and genomic feature mapping percentages were identified, as well as total genes discovered as criterion for evaluating sequencing quality. The dynamic range of expression and expression patterns in well-known housekeeping genes was also examined, and various markers of DNA contamination, sample degradation, and/or bias as a percentage of exonic (more than 55%), and intergenic mapping (less than 5%) as characteristics of the multiomics RNA fraction were computed. Another important metric for measuring the quality of single cell experiments was the number of genes found (>0 counts) per cell. For NA12878 cells there was an average of approximately 2500, whereas the average number of HBRR and UHRR genes discovered was around 6 and 7 thousand, respectively. Lastly, median absolute deviation (MAD) and percent coefficient of variation (CV) scores were calculated on normalized CPM values for general use housekeeping genes for cross-tissue studies. These metrics measure reproducibility and are robust approaches to measuring sample variability. Overall, comparable monotonous expression metrics across housekeeping genes of examined, as well as MAD values ranging from 0.25 to 1 for our HBRR and UHRR benchmarks were observed, suggesting these genes exhibit little variability in expression across cells. NA12878, demonstrated slightly more irregularity, which without being bound by theory may imply higher variability or unsuitable housekeeping genes. Correspondingly, CV rates varied from 14 to 30 percent, despite NA12878 exhibiting more variation. For each cell, the dynamic range of expressed genes was around 1300 (HBRR), 1400 (UHRR), and 1900 (NA12878) CPM.

A comparison of multiomics full-transcript performance vs. an amalgam of publicly-available bulk RNA-Seq and 3′ end-counting datasets was obtained (See Methods), highlighting the increased 5′ UTR and gene body coverage that occurs by definition relative to 3′ end-counting. The relative types of other RNA species detected with the multiomics chemistry, including lncRNAs, snRNAs, and pseudogenes are shown. Relative proportions of features were concordant between the template-switching RT chemistry in isolation vs. in the combined RNA/DNA workflow in multiomics, and overall concordance was observed between purified RNA input template vs. single cells, with the exception that single cells revealed more intronic reads of protein coding genes than did the purified RNA input. In all single cells analyzed in Tables 1B and 1C, mitochondrial read percentage was <10%, with most cells averaging less than 5%, indicating that single-cell lysis was optimal for capturing mRNA and other polyadenylated transcripts and that the amplified cells were healthy.

TABLE 1B

| | | | total. | reads. | total. | | | | overlapping. |
|---|---|---|---|---|---|---|---|---|---|
| bulk_sc | molecule | cell_line | input | aligned | alignments | exonic | intronic | intergenic | exon |
| bulk | RNA | NA12878 | 111982 | 106846 | 141318 | 67871 | 22726 | 2728 | 3643 |
| bulk | RNA | NA12878 | 160782 | 142094 | 154588 | 18529 | 73521 | 43815 | 3024 |
| bulk | RNA | NA12878 | 140426 | 133910 | 182310 | 10526 | 7405 | 988 | 3999 |
| bulk | RNA | NA12878 | 129376 | 124326 | 160948 | 98690 | 6704 | 831 | 3884 |
| bulk | RNA | NA12878 | 136722 | 131808 | 169050 | 105443 | 6197 | 961 | 4125 |
| bulk | RNA | NA12878 | 135112 | 130128 | 166204 | 103493 | 6913 | 913 | 4057 |
| sc | RNA | NA12878 | 122118 | 114636 | 149060 | 78748 | 18269 | 3057 | 3871 |
| sc | RNA | NA12878 | 121338 | 117168 | 151682 | 83646 | 16463 | 1883 | 3983 |
| sc | RNA | NA12878 | 118964 | 112087 | 152697 | 76246 | 16658 | 2685 | 3604 |
| sc | RNA | NA12878 | 110644 | 106639 | 136866 | 77158 | 14263 | 1407 | 3578 |
| sc | RNA | NA12878 | 143912 | 136541 | 169835 | 99176 | 17745 | 2444 | 4568 |
| sc | RNA | NA12878 | 121504 | 115879 | 150802 | 74453 | 24615 | 3135 | 3931 |
| sc | RNA | NA12878 | 144626 | 136420 | 165574 | 81838 | 31608 | 8792 | 4293 |
| sc | RNA | NA12878 | 130496 | 123579 | 151006 | 85721 | 19493 | 3093 | 4249 |
| sc | RNA | NA12878 | 118214 | 113371 | 144518 | 81924 | 14857 | 2147 | 3597 |
| sc | RNA | NA12878 | 129322 | 124358 | 163340 | 88167 | 18044 | 2007 | 4039 |
| sc | RNA | NA12878 | 132124 | 125034 | 149716 | 78679 | 24883 | 8253 | 4004 |
| sc | RNA | NA12878 | 128216 | 122825 | 156627 | 83410 | 21313 | 2815 | 4071 |
| sc | RNA | NA12878 | 124238 | 115933 | 146241 | 56731 | 35040 | 11854 | 3145 |
| sc | RNA | NA12878 | 112982 | 108011 | 151101 | 70197 | 19180 | 3443 | 3637 |
| sc | RNA | NA12878 | 128944 | 120676 | 161147 | 71217 | 25221 | 8907 | 3526 |
| sc | RNA | NA12878 | 148522 | 140398 | 188764 | 96096 | 22268 | 2912 | 4718 |
| sc | RNA | HBRR | 142396 | 133160 | 189868 | 72659 | 40530 | 4206 | 3976 |
| sc | RNA | HBRR | 140702 | 133160 | 165570 | 78957 | 37160 | 4146 | 4286 |
| sc | RNA | HBRR | 142816 | 134463 | 192603 | 72971 | 41155 | 4073 | 3988 |
| sc | RNA | HBRR | 142896 | 134357 | 183723 | 73477 | 41903 | 4159 | 4032 |
| sc | RNA | HBRR | 148176 | 138925 | 192628 | 77596 | 41405 | 4078 | 4230 |
| sc | RNA | HBRR | 148120 | 139804 | 192584 | 74651 | 45370 | 4269 | 3990 |
| sc | RNA | UHRR | 153988 | 146990 | 179179 | 101870 | 25508 | 2753 | 4746 |
| sc | RNA | UHRR | 158422 | 151785 | 178137 | 100862 | 32054 | 3232 | 4797 |
| sc | RNA | UHRR | 153772 | 146590 | 173628 | 103020 | 24343 | 2590 | 4822 |
| sc | RNA | UHRR | 158164 | 148954 | 179554 | 102533 | 26983 | 2898 | 4729 |
| sc | RNA | HBRR | 137574 | 128871 | 192359 | 65776 | 42080 | 4455 | 3444 |
| sc | RNA | HBRR | 140364 | 132257 | 158163 | 71291 | 45036 | 4346 | 3587 |
| sc | RNA | HBRR | 139028 | 131062 | 180890 | 68430 | 43716 | 4363 | 3491 |
| bulk | RNA | NA12878 | 143978 | 136784 | 216400 | 103723 | 6164 | 991 | 4093 |
| bulk | RNA | NA12878 | 143722 | 136331 | 224271 | 102199 | 6450 | 1001 | 3977 |
| sc | RNA | NA12878 | 144980 | 135564 | 228417 | 83320 | 24918 | 2786 | 4005 |
| sc | RNA | NA12878 | 140544 | 137523 | 178383 | 84321 | 33982 | 2346 | 4209 |
| sc | RNA | NA12878 | 145180 | 132673 | 168014 | 80819 | 32462 | 4271 | 3987 |
| sc | RNA | NA12878 | 143670 | 137319 | 156855 | 88397 | 31651 | 3398 | 4290 |
| sc | RNA | NA12878 | 134768 | 133779 | 157409 | 79499 | 36392 | 4688 | 3989 |
| sc | RNA | NA12878 | 137936 | 128515 | 189755 | 76312 | 30484 | 3555 | 3730 |
| sc | RNA | NA12878 | 144234 | 131374 | 216266 | 83585 | 21947 | 2279 | 4209 |
| sc | RNA | NA12878 | 132162 | 124697 | 226673 | 72121 | 25851 | 2027 | 3676 |
| sc | RNA | NA12878 | 133524 | 126383 | 185224 | 69435 | 35414 | 4116 | 3683 |
| sc | RNA | NA12878 | 143500 | 135461 | 182395 | 83406 | 31021 | 3717 | 4465 |
| sc | RNA | NA12878 | 151438 | 140328 | 165606 | 91145 | 30500 | 2840 | 4505 |
| sc | RNA | NA12878 | 130816 | 123078 | 147245 | 74934 | 32092 | 4608 | 3835 |
| sc | RNA | NA12878 | 127082 | 120176 | 240436 | 67613 | 22962 | 2354 | 3415 |
| sc | RNA | UHRR | 138566 | 131354 | 222876 | 83525 | 20513 | 2425 | 4140 |
| sc | RNA | UHRR | 148376 | 141908 | 210176 | 93082 | 24140 | 2672 | 4415 |
| sc | RNA | UHRR | 149260 | 142977 | 197463 | 94311 | 25247 | 2927 | 4306 |
| sc | RNA | HBRR | 149692 | 141335 | 205105 | 77382 | 42238 | 4414 | 4244 |
| sc | RNA | HBRR | 148820 | 140776 | 193856 | 84758 | 35262 | 3041 | 4357 |
| sc | RNA | HBRR | 145386 | 138027 | 192103 | 77421 | 40470 | 4013 | 4309 |
| sc | RNA | UHRR | 163098 | 157847 | 181373 | 106155 | 33267 | 3030 | 5141 |
| sc | RNA | UHRR | 157622 | 151952 | 181530 | 107047 | 25235 | 2539 | 4958 |
| sc | RNA | UHRR | 160140 | 151850 | 179606 | 108116 | 24306 | 2431 | 5080 |
| sc | RNA | NA12878 | 126142 | 117944 | 156564 | 78164 | 20970 | 2337 | 3777 |
| sc | RNA | NA12878 | 141286 | 131697 | 209189 | 89423 | 16523 | 2415 | 4261 |
| sc | RNA | NA12878 | 146528 | 137691 | 186618 | 93643 | 21118 | 2372 | 4708 |
| sc | RNA | NA12878 | 138288 | 129261 | 174250 | 81689 | 27719 | 3284 | 4171 |
| sc | RNA | NA12878 | 144914 | 136295 | 188013 | 88923 | 24216 | 2963 | 4756 |
| sc | RNA | NA12878 | 133040 | 125035 | 172667 | 78929 | 26431 | 1292 | 4339 |
| sc | RNA | NA12878 | 146740 | 135709 | 176506 | 91173 | 25333 | 3479 | 4071 |
| sc | RNA | NA12878 | 138130 | 129626 | 172454 | 92768 | 16749 | 1580 | 4091 |
| bulk | RNA | NA12878 | 100760 | 92681 | 184311 | 62633 | 6971 | 868 | 2744 |
| bulk | RNA | NA12878 | 107446 | 99225 | 202576 | 66537 | 7103 | 801 | 3034 |
| sc | RNA | NA12878 | 132740 | 125738 | 167834 | 90236 | 16007 | 2010 | 4337 |
| sc | RNA | NA12878 | 131602 | 124040 | 158900 | 83280 | 21667 | 2546 | 4102 |
| sc | RNA | NA12878 | 128620 | 121367 | 169355 | 82838 | 18594 | 1854 | 4107 |
| sc | RNA | NA12878 | 126352 | 119347 | 162791 | 86849 | 13847 | 1381 | 3357 |
| sc | RNA | NA12878 | 134156 | 126688 | 176754 | 92174 | 13631 | 1532 | 3967 |
| sc | RNA | NA12878 | 125816 | 118443 | 159207 | 78562 | 20826 | 2490 | 4458 |
| sc | RNA | NA12878 | 132586 | 124957 | 152707 | 91420 | 15639 | 1502 | 4124 |
| sc | RNA | NA12878 | 118670 | 111002 | 156832 | 74414 | 17292 | 2715 | 3701 |
| bulk | RNA | NA12878 | 138622 | 133430 | 165617 | 103108 | 12545 | 1525 | 4215 |
| bulk | RNA | NA12878 | 96724 | 92938 | 114088 | 72165 | 8489 | 1061 | 3024 |
| sc | RNA | NA12878 | 113328 | 108475 | 151609 | 72767 | 17461 | 3083 | 3460 |
| sc | RNA | NA12878 | 102354 | 97609 | 122702 | 64882 | 19071 | 2260 | 3330 |
| sc | RNA | NA12878 | 110128 | 105336 | 122053 | 75753 | 17225 | 1789 | 3311 |
| sc | RNA | NA12878 | 110028 | 105739 | 130703 | 75741 | 16065 | 1885 | 3288 |
| sc | RNA | NA12878 | 90036 | 86032 | 104630 | 60934 | 13870 | 1487 | 2601 |
| sc | RNA | NA12878 | 104292 | 99673 | 126801 | 67364 | 18530 | 1640 | 3208 |
| sc | RNA | NA12878 | 77024 | 73031 | 89320 | 50079 | 12920 | 1350 | 2370 |
| sc | RNA | NA12878 | 80158 | 75682 | 88106 | 50423 | 15589 | 1626 | 2524 |
| sc | RNA | NA12878 | 79398 | 75557 | 101959 | 50129 | 13513 | 2067 | 2440 |
| sc | RNA | NA12878 | 95670 | 91545 | 116468 | 58654 | 20393 | 2076 | 2811 |
| sc | RNA | NA12878 | 116776 | 112440 | 138904 | 76132 | 21116 | 2262 | 3653 |
| sc | RNA | NA12878 | 107766 | 103440 | 133453 | 69993 | 18893 | 1928 | 3096 |
| sc | RNA | NA12878 | 102684 | 98505 | 126691 | 59018 | 24165 | 4366 | 3778 |
| sc | RNA | NA12878 | 101714 | 97289 | 120383 | 59214 | 25036 | 2506 | 3118 |
| sc | RNA | NA12878 | 126382 | 121125 | 147308 | 82514 | 23359 | 2213 | 3569 |
| sc | RNA | NA12878 | 126314 | 121195 | 156459 | 87591 | 16410 | 1632 | 3517 |
| sc | RNA | NA12878 | 116218 | 108082 | 183166 | 72804 | 13054 | 1517 | 3132 |
| sc | RNA | NA12878 | 118604 | 110813 | 160513 | 68867 | 20379 | 5017 | 4363 |
| sc | RNA | NA12878 | 120336 | 112402 | 161540 | 76312 | 16318 | 1762 | 3795 |
| sc | RNA | NA12878 | 110522 | 102852 | 172510 | 67106 | 13709 | 2278 | 3416 |
| sc | RNA | NA12878 | 114190 | 107902 | 152723 | 74024 | 14981 | 2584 | 3488 |
| sc | RNA | NA12878 | 116670 | 107937 | 136282 | 69313 | 23128 | 2899 | 3485 |
| sc | RNA | NA12878 | 110966 | 103027 | 135605 | 73093 | 13293 | 2035 | 3510 |
| sc | RNA | NA12878 | 107620 | 99223 | 131167 | 72053 | 9809 | 1831 | 3270 |
| sc | RNA | NA12878 | 114226 | 105769 | 170667 | 67855 | 16492 | 1819 | 3199 |
| sc | RNA | NA12878 | 116384 | 106922 | 164565 | 71262 | 14228 | 2758 | 3424 |
| sc | RNA | NA12878 | 107744 | 99961 | 158155 | 69215 | 10695 | 1864 | 3407 |
| sc | RNA | NA12878 | 116598 | 108628 | 148294 | 73968 | 16704 | 3017 | 3237 |
| sc | RNA | NA12878 | 110282 | 103075 | 137635 | 69704 | 15205 | 3778 | 3568 |
| sc | RNA | NA12878 | 118408 | 109895 | 138791 | 72362 | 20209 | 2768 | 3659 |
| sc | RNA | NA12878 | 117148 | 107397 | 143382 | 66036 | 24216 | 3056 | 3757 |
| sc | RNA | NA12878 | 118866 | 108362 | 155308 | 66890 | 21653 | 4367 | 3548 |
| sc | RNA | NA12878 | 116610 | 105645 | 157171 | 68813 | 16598 | 3146 | 3144 |
| sc | RNA | NA12878 | 125668 | 113642 | 156008 | 73868 | 19276 | 4188 | 3532 |
| sc | RNA | NA12878 | 120784 | 110723 | 163032 | 73717 | 17637 | 2464 | 3544 |
| sc | RNA | NA12878 | 114684 | 103595 | 137658 | 64496 | 22441 | 3273 | 3251 |
| sc | RNA | NA12878 | 110700 | 100357 | 145931 | 71217 | 11485 | 1519 | 3008 |
| sc | RNA | NA12878 | 111690 | 103308 | 156098 | 63508 | 20221 | 3325 | 3057 |
| sc | RNA | NA12878 | 122000 | 112154 | 141235 | 76596 | 18234 | 2675 | 3902 |
| sc | RNA | NA12878 | 119194 | 109972 | 149290 | 73169 | 17588 | 3478 | 3458 |
| sc | RNA | NA12878 | 119014 | 110267 | 142615 | 75842 | 16573 | 2929 | 3661 |
| sc | RNA | NA12878 | 114046 | 104513 | 145301 | 66024 | 19688 | 3668 | 3518 |
| sc | RNA | NA12878 | 127530 | 120122 | 157982 | 86848 | 14502 | 2090 | 4098 |
| sc | RNA | NA12878 | 104572 | 94766 | 175564 | 50996 | 22803 | 2312 | 2903 |
| sc | RNA | NA12878 | 119448 | 108317 | 139138 | 74790 | 15640 | 2840 | 3772 |
| sc | RNA | NA12878 | 118904 | 103913 | 145759 | 35008 | 51416 | 6701 | 2536 |
| sc | RNA | NA12878 | 119136 | 111326 | 153893 | 76442 | 17317 | 2126 | 3408 |
| sc | RNA | NA12878 | 109002 | 99049 | 152519 | 55166 | 25830 | 2828 | 2769 |
| sc | RNA | NA12878 | 106770 | 97327 | 122323 | 62465 | 20407 | 2740 | 2978 |
| sc | RNA | NA12878 | 110346 | 101914 | 121316 | 70419 | 17942 | 1943 | 2990 |
| sc | RNA | NA12878 | 135554 | 124403 | 157873 | 79492 | 25168 | 4540 | 3854 |
| sc | RNA | NA12878 | 120270 | 112391 | 153542 | 75968 | 18376 | 2244 | 3177 |
| sc | RNA | NA12878 | 118782 | 107713 | 138224 | 65138 | 26804 | 3877 | 3175 |
| sc | RNA | HBRR | 137840 | 122472 | 224322 | 59916 | 37062 | 4033 | 3184 |
| sc | RNA | HBRR | 141724 | 128960 | 226946 | 63924 | 39646 | 4203 | 3487 |
| sc | RNA | HBRR | 134784 | 119371 | 235263 | 59597 | 32964 | 3512 | 3288 |
| sc | RNA | UHRR | 140734 | 128770 | 191224 | 81969 | 23691 | 2630 | 3896 |
| sc | RNA | UHRR | 140658 | 130183 | 189551 | 84169 | 23234 | 2397 | 3898 |
| sc | RNA | UHRR | 146628 | 133046 | 221912 | 81913 | 24338 | 2819 | 3912 |
| sc | RNA | HBRR | 136628 | 125022 | 205062 | 62321 | 40136 | 4255 | 3395 |
| sc | RNA | HBRR | 135382 | 123994 | 207964 | 60650 | 40388 | 4204 | 3291 |
| sc | RNA | HBRR | 90 | 80 | 122 | 52 | 20 | 0 | 3 |
| sc | RNA | UHRR | 137672 | 126694 | 188278 | 81587 | 22468 | 2679 | 3702 |
| sc | RNA | UHRR | 141402 | 131800 | 196341 | 84061 | 23823 | 2776 | 4032 |
| sc | RNA | UHRR | 144448 | 132149 | 200465 | 82133 | 26780 | 2942 | 3795 |
| sc | RNA | HBRR | 113834 | 104116 | 156574 | 54317 | 32530 | 3552 | 2805 |
| sc | RNA | HBRR | 131850 | 117908 | 171888 | 61530 | 37539 | 4065 | 3046 |
| sc | RNA | HBRR | 129554 | 116980 | 174938 | 59221 | 38515 | 4034 | 2915 |
| sc | RNA | UHRR | 147708 | 136874 | 199646 | 92942 | 19407 | 2394 | 4077 |
| sc | RNA | UHRR | 144330 | 134765 | 188675 | 93265 | 17594 | 2363 | 4218 |
| sc | RNA | UHRR | 146428 | 131089 | 202553 | 88106 | 17343 | 2233 | 3978 |
| sc | RNA | HBRR | 130786 | 121305 | 178129 | 64920 | 37511 | 3789 | 3650 |
| sc | RNA | HBRR | 155292 | 142202 | 199334 | 70564 | 50917 | 4997 | 3991 |
| sc | RNA | HBRR | 136016 | 126734 | 186320 | 67722 | 39148 | 4025 | 3710 |
| sc | RNA | UHRR | 160566 | 154019 | 183197 | 99487 | 36235 | 3240 | 4615 |
| sc | RNA | UHRR | 152090 | 144216 | 171512 | 98217 | 27532 | 2906 | 4415 |
| sc | RNA | UHRR | 152704 | 144822 | 188846 | 97957 | 25617 | 2930 | 4445 |
| sc | RNA | HBRR | 109206 | 100261 | 150237 | 54303 | 29651 | 2865 | 3026 |
| sc | RNA | HBRR | 108644 | 98954 | 155633 | 50367 | 31288 | 3265 | 3009 |
| sc | RNA | HBRR | 109950 | 100546 | 159716 | 50590 | 32561 | 3195 | 2861 |
| sc | RNA | UHRR | 126138 | 116638 | 153232 | 80558 | 17749 | 1966 | 3833 |
| sc | RNA | UHRR | 128772 | 120228 | 156904 | 82815 | 19354 | 2014 | 4104 |
| sc | RNA | UHRR | 136726 | 126628 | 159514 | 85577 | 22942 | 2476 | 4080 |
| sc | RNA | HBRR | 138318 | 129024 | 167190 | 69010 | 42614 | 4572 | 3792 |
| sc | RNA | HBRR | 139380 | 129432 | 178906 | 66904 | 43664 | 4392 | 3645 |
| sc | RNA | HBRR | 145512 | 135581 | 184305 | 71099 | 45442 | 4564 | 3793 |
| sc | RNA | UHRR | 159122 | 151246 | 183888 | 105483 | 25080 | 2875 | 4919 |
| sc | RNA | UHRR | 152006 | 143872 | 173380 | 99883 | 24394 | 2787 | 4705 |
| sc | RNA | UHRR | 153664 | 146284 | 175794 | 101498 | 25274 | 2530 | 4845 |
| sc | RNA | HBRR | 134420 | 125450 | 173204 | 65346 | 42022 | 4383 | 3477 |
| sc | RNA | HBRR | 147664 | 134924 | 204874 | 66295 | 46658 | 4611 | 3761 |
| sc | RNA | HBRR | 140818 | 130446 | 180226 | 66436 | 45287 | 4519 | 3557 |
| sc | RNA | UHRR | 155146 | 146944 | 171188 | 100702 | 27965 | 2913 | 4599 |
| sc | RNA | UHRR | 148482 | 140838 | 167348 | 98067 | 24498 | 2610 | 4512 |
| sc | RNA | UHRR | 144666 | 137465 | 164347 | 94877 | 24126 | 2554 | 4621 |
| sc | RNA | HBRR | 128916 | 112136 | 174807 | 57495 | 35065 | 3853 | 3134 |
| sc | RNA | HBRR | 130106 | 113057 | 179909 | 56796 | 36521 | 3722 | 3159 |
| sc | RNA | HBRR | 143404 | 130388 | 215337 | 64686 | 41370 | 4554 | 3657 |
| bulk | RNA | NA12878 | 143350 | 126900 | 179425 | 99062 | 6312 | 942 | 3979 |
| bulk | RNA | NA12878 | 141664 | 117129 | 167179 | 90528 | 5900 | 932 | 3699 |
| sc | RNA | NA12878 | 132590 | 127675 | 162095 | 93353 | 14085 | 1590 | 4554 |
| sc | RNA | NA12878 | 142098 | 122816 | 181668 | 85002 | 11528 | 2564 | 4235 |
| sc | RNA | NA12878 | 139124 | 129376 | 174528 | 91028 | 13165 | 2659 | 4751 |
| sc | RNA | NA12878 | 131144 | 125538 | 165028 | 94165 | 11649 | 1492 | 4125 |
| sc | RNA | NA12878 | 148550 | 117786 | 169358 | 79033 | 18028 | 2431 | 3947 |
| sc | RNA | NA12878 | 144128 | 129895 | 179340 | 93403 | 13926 | 1862 | 4367 |
| sc | RNA | NA12878 | 138168 | 131439 | 157113 | 93749 | 18724 | 1939 | 4471 |
| sc | RNA | NA12878 | 141168 | 129360 | 198168 | 88944 | 14198 | 1810 | 4569 |
| sc | RNA | NA12878 | 126778 | 113809 | 229231 | 70930 | 14810 | 1684 | 3450 |
| sc | RNA | NA12878 | 148272 | 127713 | 164545 | 93937 | 13748 | 1764 | 4125 |
| sc | RNA | NA12878 | 123396 | 111943 | 157058 | 73739 | 18668 | 2914 | 3776 |
| sc | RNA | NA12878 | 131706 | 119684 | 201722 | 82145 | 11446 | 1559 | 3971 |
| sc | RNA | NA12878 | 129742 | 118824 | 187066 | 85268 | 8156 | 1574 | 4137 |
| sc | RNA | NA12878 | 127436 | 112675 | 222674 | 67891 | 15663 | 1801 | 3398 |
| sc | RNA | NA12878 | 149770 | 138698 | 253538 | 83851 | 16896 | 3672 | 4995 |
| sc | RNA | NA12878 | 142150 | 128962 | 183466 | 91323 | 12276 | 2046 | 4652 |
| sc | RNA | UHRR | 159332 | 126409 | 158970 | 85888 | 22087 | 2471 | 3991 |
| sc | RNA | UHRR | 148282 | 127745 | 157771 | 88996 | 21063 | 2312 | 4099 |
| sc | RNA | UHRR | 141896 | 122444 | 153106 | 83927 | 20795 | 2153 | 4014 |

TABLE 1C

| | | | | | overlapping. | | | |
|---|---|---|---|---|---|---|---|---|
| | reads. | exonic_Percent | intronic_Percent | intergenic_Percent | exon_Percent | | | |
| | aligned_Percent | of Genomic | of Genomic | of Genomic | of Genomic | | | |
| sample_name | Input Total | Alignments | Alignments | Alignments | Alignments | X5..bias | X3..bias | X5..3..bias |
| H3L73C- | 95.41354861 | 69.99319363 | 23.43659764 | 2.813299233 | 3.756909496 | 0.67 | 100 | 100 |
| NA12878- | | | | | | | | |
| RNA- | | | | | | | | |
| 100pg | | | | | | | | |
| H3L73C- | 88.37680835 | 13.34086933 | 52.93507765 | 31.54677476 | 2.177278258 | 0.74 | 100 | 100 |
| NA12878- | | | | | | | | |
| RNA-NTC | | | | | | | | |
| H3L73C- | 95.35983365 | 89.46795852 | 6.293557709 | 0.839707632 | 3.398776135 | 0.74 | 100 | 100 |
| NA12878- | | | | | | | | |
| RNA- | | | | | | | | |
| 100pg | | | | | | | | |
| H3L73C- | 96.09664853 | 89.62936726 | 6.088512292 | 0.7547067 | 3.527413745 | 0.75 | 100 | 100 |
| NA12878- | | | | | | | | |
| RNA- | | | | | | | | |
| 100pg | | | | | | | | |
| H3L73C- | 96.40584544 | 90.33377311 | 5.309014273 | 0.823295581 | 3.533917036 | 0.72 | 100 | 100 |
| NA12878- | | | | | | | | |
| RNA- | | | | | | | | |
| 100pg | | | | | | | | |
| H3L73C- | 96.31120848 | 89.70063098 | 5.991714048 | 0.791325752 | 3.516329219 | 0.74 | 100 | 100 |
| NA12878- | | | | | | | | |
| RNA- | | | | | | | | |
| 100pg | | | | | | | | |
| H3L73C- | 93.87313909 | 75.75929578 | 17.57564096 | 2.940978402 | 3.724084853 | 0.79 | 100 | 100 |
| NA12878- | | | | | | | | |
| RNA-SC2 | | | | | | | | |
| H3L73C- | 96.56331899 | 78.92993631 | 15.53479594 | 1.776834159 | 3.758433593 | 0.74 | 100 | 100 |
| NA12878- | | | | | | | | |
| RNA-SC2 | | | | | | | | |
| H3L73C- | 94.21925961 | 76.86631113 | 16.79352374 | 2.706844233 | 3.6333209 | 0.74 | 100 | 100 |
| NA12878- | | | | | | | | |
| RNA-SC3 | | | | | | | | |
| H3L73C- | 96.38028271 | 80.03443769 | 14.79472232 | 1.459452731 | 3.711387258 | 0.76 | 100 | 100 |
| NA12878- | | | | | | | | |
| RNA-SC3 | | | | | | | | |
| H3L73C- | 94.87811996 | 80.02388387 | 14.31822033 | 1.972033276 | 3.685862522 | 0.76 | 100 | 100 |
| NA12878- | | | | | | | | |
| RNA-SC4 | | | | | | | | |
| H3L73C- | 95.37052278 | 70.14999906 | 23.19237944 | 2.953813104 | 3.703808393 | 0.74 | 100 | 100 |
| NA12878- | | | | | | | | |
| RNA-SC4 | | | | | | | | |
| H3L73C- | 94.32605479 | 64.67822115 | 24.98043958 | 6.948494835 | 3.392844441 | 0.74 | 100 | 100 |
| NA12878- | | | | | | | | |
| RNA-SC5 | | | | | | | | |
| H3L73C- | 94.69945439 | 76.15853442 | 17.31849035 | 2.747965457 | 3.775009773 | 0.71 | 100 | 100 |
| NA12878- | | | | | | | | |
| RNA-SC5 | | | | | | | | |
| H3L73C- | 95.90319252 | 79.9063643 | 14.49109973 | 2.094123385 | 3.508412582 | 0.72 | 100 | 100 |
| NA12878- | | | | | | | | |
| RNA-SC5 | | | | | | | | |
| H3L73C- | 96.16151931 | 78.54031374 | 16.07383059 | 1.787861781 | 3.597993889 | 0.74 | 100 | 100 |
| NA12878- | | | | | | | | |
| RNA-SC5 | | | | | | | | |
| H3L73C- | 94.63382883 | 67.93272261 | 21.48438512 | 7.125773837 | 3.457118435 | 0.71 | 100 | 100 |
| NA12878- | | | | | | | | |
| RNA-SC6 | | | | | | | | |
| H3L73C- | 95.79537655 | 74.73411642 | 19.09613024 | 2.522198031 | 3.647555305 | 0.76 | 100 | 100 |
| NA12878- | | | | | | | | |
| RNA-SC6 | | | | | | | | |
| H3L73C- | 93.31524976 | 53.13383909 | 32.81820736 | 11.10236958 | 2.945583966 | 0.78 | 100 | 100 |
| NA12878- | | | | | | | | |
| RNA-SC7 | | | | | | | | |
| H3L73C- | 95.6001841 | 72.77543361 | 19.88450812 | 3.569466187 | 3.770592077 | 0.7 | 100 | 100 |
| NA12878- | | | | | | | | |
| RNA-SC7 | | | | | | | | |
| H3L73C- | 93.58791413 | 65.41411395 | 23.16594869 | 8.18124202 | 3.238695337 | 0.7 | 100 | 100 |
| NA12878- | | | | | | | | |
| RNA-SC8 | | | | | | | | |
| H3L73C- | 94.53010328 | 76.27029859 | 17.67385749 | 2.311221169 | 3.74462276 | 0.73 | 100 | 100 |
| NA12878- | | | | | | | | |
| RNA-SC8 | | | | | | | | |
| H3LK3W- | 93.51386275 | 59.86520668 | 33.3934795 | 3.465407717 | 3.275906106 | 0.68 | 100 | 100 |
| HBRR | | | | | | | | |
| H3LK3W- | 94.63973504 | 63.39432673 | 29.83564701 | 3.328810348 | 3.441215907 | 0.71 | 100 | 100 |
| HBRR | | | | | | | | |
| H3LK3W- | 94.15121555 | 59.72075589 | 33.68197926 | 3.333415175 | 3.263849673 | 0.61 | 100 | 100 |
| HBRR | | | | | | | | |
| H3LK3W- | 94.02432538 | 59.46136229 | 33.9100598 | 3.365676413 | 3.26290149 | 0.71 | 100 | 100 |
| HBRR | | | | | | | | |
| H3LK3W- | 93.75674873 | 60.9509147 | 32.52323088 | 3.203229937 | 3.322624481 | 0.67 | 100 | 100 |
| HBRR | | | | | | | | |
| H3LK3W- | 94.38563327 | 58.19379482 | 35.36794512 | 3.32787652 | 3.110383536 | 0.65 | 100 | 100 |
| HBRR | | | | | | | | |
| H3LK3W- | 95.45549004 | 75.52807373 | 18.91204579 | 2.041118945 | 3.518761538 | 0.69 | 100 | 100 |
| HBRR | | | | | | | | |
| H3LK3W- | 95.81055661 | 71.5612473 | 22.74220441 | 2.29309305 | 3.403455249 | 0.76 | 100 | 100 |
| HBRR | | | | | | | | |
| H3LK3W- | 95.32944879 | 76.43850863 | 18.06195511 | 1.921721387 | 3.577814877 | 0.73 | 100 | 100 |
| HBRR | | | | | | | | |
| H3LK3W- | 94.17693027 | 74.76356795 | 19.67508367 | 2.113122799 | 3.448225575 | 0.76 | 100 | 100 |
| HBRR | | | | | | | | |
| H3LK3W- | 93.67395002 | 56.82346335 | 36.35264135 | 3.848645847 | 2.975249449 | 0.66 | 100 | 100 |
| HBRR | | | | | | | | |
| H3LK3W- | 94.22430253 | 57.37244487 | 36.2433607 | 3.497505231 | 2.8866892 | 0.71 | 100 | 100 |
| HBRR | | | | | | | | |
| H3LK3W- | 94.27021895 | 57.025 | 36.43 | 3.635833333 | 2.909166667 | 0.66 | 100 | 100 |
| HBRR | | | | | | | | |
| H3LK3W- | 95.0034033 | 90.21666333 | 5.361351993 | 0.861956493 | 3.560028181 | 0.71 | 100 | 100 |
| NA12878- | | | | | | | | |
| 100pg | | | | | | | | |
| H3LK3W- | 94.8574331 | 89.94253126 | 5.676467741 | 0.88095259 | 3.500048404 | 0.73 | 100 | 100 |
| NA12878- | | | | | | | | |
| 100pg | | | | | | | | |
| H3LK3W- | 93.50531108 | 72.43390797 | 21.6623634 | 2.421997931 | 3.481730694 | 0.75 | 100 | 100 |
| NA12878- | | | | | | | | |
| SC1 | | | | | | | | |
| H3LK3W- | 97.85049522 | 67.53351808 | 27.21651796 | 1.87893447 | 3.37102949 | 0.72 | 100 | 100 |
| NA12878- | | | | | | | | |
| SC11 | | | | | | | | |
| H3LK3W- | 91.38517702 | 66.49635097 | 26.70912217 | 3.514098355 | 3.280428504 | 0.74 | 100 | 100 |
| NA12878- | | | | | | | | |
| SC12 | | | | | | | | |
| H3LK3W- | 95.57945291 | 69.2028872 | 24.7784493 | 2.660174109 | 3.358489384 | 0.76 | 100 | 100 |
| NA12878- | | | | | | | | |
| SC13 | | | | | | | | |
| H3LK3W- | 99.26614627 | 63.81976109 | 29.21456554 | 3.763406332 | 3.202267035 | 0.71 | 100 | 100 |
| NA12878- | | | | | | | | |
| SC14 | | | | | | | | |
| H3LK3W- | 93.17002088 | 66.89282177 | 26.72136464 | 3.116206906 | 3.269606683 | 0.66 | 100 | 100 |
| NA12878- | | | | | | | | |
| SC15 | | | | | | | | |
| H3LK3W- | 91.08393305 | 74.61613998 | 19.59203714 | 2.034458132 | 3.757364756 | 0.73 | 100 | 100 |
| NA12878- | | | | | | | | |
| SC16 | | | | | | | | |
| H3LK3W- | 94.35162906 | 69.56450446 | 24.93465156 | 1.9551483 | 3.545695684 | 0.75 | 100 | 100 |
| NA12878- | | | | | | | | |
| SC2 | | | | | | | | |
| H3LK3W- | 94.65189779 | 61.63891059 | 31.437753 | 3.653859811 | 3.2694766 | 0.72 | 100 | 100 |
| NA12878- | | | | | | | | |
| SC3 | | | | | | | | |
| H3LK3W- | 94.39790941 | 68.02600135 | 25.3007528 | 3.031588219 | 3.641657627 | 0.72 | 100 | 100 |
| NA12878- | | | | | | | | |
| SC4 | | | | | | | | |
| H3LK3W- | 92.66366434 | 70.66051632 | 23.64524382 | 2.201721064 | 3.4925188 | 0.7 | 100 | 100 |
| NA12878- | | | | | | | | |
| SC5 | | | | | | | | |
| H3LK3W- | 94.08482143 | 64.89533987 | 27.79274091 | 3.990681482 | 3.321237735 | 0.74 | 100 | 100 |
| NA12878- | | | | | | | | |
| SC6 | | | | | | | | |
| H3LK3W- | 94.56571348 | 70.17873453 | 23.83334717 | 2.443328074 | 3.544590218 | 0.76 | 100 | 100 |
| NA12878- | | | | | | | | |
| SC8 | | | | | | | | |
| H3LK3W- | 94.79526002 | 75.5178431 | 18.5465132 | 2.192526423 | 3.743117275 | 0.74 | 100 | 100 |
| UHRR | | | | | | | | |
| H3LK3W- | 95.64080444 | 74.87953406 | 19.41935017 | 2.149482338 | 3.55163343 | 0.74 | 100 | 100 |
| UHRR | | | | | | | | |
| H3LK3W- | 95.7905668 | 74.38303981 | 19.91229661 | 2.308523476 | 3.396140105 | 0.71 | 100 | 100 |
| UHRR | | | | | | | | |
| H3LK3W- | 94.41720332 | 60.32367202 | 32.92692434 | 3.440964156 | 3.308439483 | 0.74 | 100 | 100 |
| HBRR | | | | | | | | |
| H3LK3W- | 94.59481253 | 66.51964401 | 27.67426894 | 2.386632972 | 3.41945408 | 0.72 | 100 | 100 |
| HBRR | | | | | | | | |
| H3LK3W- | 94.93830217 | 61.34154168 | 32.06484277 | 3.179545689 | 3.414069866 | 0.63 | 100 | 100 |
| HBRR | | | | | | | | |
| H3LK3W- | 96.78046328 | 71.92414274 | 22.53968684 | 2.05294289 | 3.483227524 | 0.75 | 100 | 100 |
| UHRR | | | | | | | | |
| H3LK3W- | 96.40278641 | 76.58303465 | 18.05349874 | 1.816438807 | 3.547027808 | 0.74 | 100 | 100 |
| UHRR | | | | | | | | |
| H3LK3W- | 94.82327963 | 77.26269 | 17.36974123 | 1.737259974 | 3.630308791 | 0.74 | 100 | 100 |
| UHRR | | | | | | | | |
| H3LK7N- | 93.50097509 | 74.26649438 | 19.92436911 | 2.220469748 | 3.588666768 | 0.83 | 100 | 100 |
| NA12878- | | | | | | | | |
| SC17 | | | | | | | | |
| H3LK7N- | 93.2130572 | 79.40100513 | 14.67120101 | 2.144341248 | 3.783452611 | 0.76 | 100 | 100 |
| NA12878- | | | | | | | | |
| SC18 | | | | | | | | |
| H3LK7N- | 93.96907076 | 76.85672311 | 17.33242505 | 1.946799517 | 3.86405233 | 0.7 | 100 | 100 |
| NA12878- | | | | | | | | |
| SC19 | | | | | | | | |
| H3LK7N- | 93.47231864 | 69.9015086 | 23.71922679 | 2.810128099 | 3.56913651 | 0.74 | 100 | 100 |
| NA12878- | | | | | | | | |
| SC20 | | | | | | | | |
| H3LK7N- | 94.05233449 | 73.57642854 | 20.03673733 | 2.451637459 | 3.935196677 | 0.76 | 100 | 100 |
| NA12878- | | | | | | | | |
| SC21 | | | | | | | | |
| H3LK7N- | 93.98301263 | 71.11297312 | 23.81364255 | 1.164058347 | 3.909325981 | 0.74 | 100 | 100 |
| NA12878- | | | | | | | | |
| SC22 | | | | | | | | |
| H3LK7N- | 92.48262233 | 73.49342233 | 20.4206165 | 2.804378668 | 3.281582511 | 0.72 | 100 | 100 |
| NA12878- | | | | | | | | |
| SC23 | | | | | | | | |
| H3LK7N- | 93.84348078 | 80.53616696 | 14.54057714 | 1.37167066 | 3.551585235 | 0.78 | 100 | 100 |
| NA12878- | | | | | | | | |
| SC24 | | | | | | | | |
| H3LK7N- | 91.98193728 | 85.54550918 | 9.52114292 | 1.185533217 | 3.747814685 | 0.76 | 100 | 100 |
| NA12878- | | | | | | | | |
| 100pg | | | | | | | | |
| H3LK7N- | 92.3487147 | 85.88189739 | 9.168118748 | 1.033881897 | 3.916101968 | 0.68 | 100 | 100 |
| NA12878- | | | | | | | | |
| 100pg | | | | | | | | |
| H3LK7N- | 94.72502637 | 80.14566125 | 14.21707079 | 1.785238476 | 3.852029488 | 0.79 | 100 | 100 |
| NA12878- | | | | | | | | |
| SC1 | | | | | | | | |
| H3LK7N- | 94.25388672 | 74.62699942 | 19.41574443 | 2.281464223 | 3.675791926 | 0.75 | 100 | 100 |
| NA12878- | | | | | | | | |
| SC2 | | | | | | | | |
| H3LK7N- | 94.3609081 | 77.13538126 | 17.31397763 | 1.726369503 | 3.824271601 | 0.79 | 100 | 100 |
| NA12878- | | | | | | | | |
| SC3 | | | | | | | | |
| H3LK7N- | 94.45596429 | 82.37285885 | 13.1333346 | 1.309824155 | 3.183982397 | 0.69 | 100 | 100 |
| NA12878- | | | | | | | | |
| SC4 | | | | | | | | |
| H3LK7N- | 94.43334625 | 82.81283692 | 12.24663983 | 1.376410551 | 3.5641127 | 0.74 | 100 | 100 |
| NA12878- | | | | | | | | |
| SC5 | | | | | | | | |
| H3LK7N- | 94.13985503 | 73.88090581 | 19.58508878 | 2.341634066 | 4.192371351 | 0.79 | 100 | 100 |
| NA12878- | | | | | | | | |
| SC6 | | | | | | | | |
| H3LK7N- | 94.24599882 | 81.1288104 | 13.87851089 | 1.3329192 | 3.659759507 | 0.79 | 100 | 100 |
| NA12878- | | | | | | | | |
| SC7 | | | | | | | | |
| H3LK7N- | 93.53838375 | 75.83824219 | 17.62295917 | 2.766963576 | 3.771835062 | 0.79 | 100 | 100 |
| NA12878- | | | | | | | | |
| SC8 | | | | | | | | |
| H3LVCY- | 96.25456277 | 84.93735224 | 10.33420378 | 1.25625036 | 3.472193619 | 0.72 | 100 | 100 |
| NA12878- | | | | | | | | |
| 100pg | | | | | | | | |
| H3LVCY- | 96.08576982 | 85.16149589 | 10.01781942 | 1.252079916 | 3.568604775 | 0.69 | 100 | 100 |
| NA12878- | | | | | | | | |
| 100pg | | | | | | | | |
| H3LVCY- | 95.71773966 | 75.1950481 | 18.04362877 | 3.1858718 | 3.575451323 | 0.73 | 100 | 100 |
| NA12878- | | | | | | | | |
| SC1 | | | | | | | | |
| H3LVCY- | 95.36412842 | 72.45904202 | 21.29814726 | 2.523927052 | 3.718883665 | 0.8 | 100 | 100 |
| NA12878- | | | | | | | | |
| SC10 | | | | | | | | |
| H3LVCY- | 95.64869969 | 77.23750484 | 17.56255225 | 1.824058402 | 3.3758845 | 0.75 | 100 | 100 |
| NA12878- | | | | | | | | |
| SC11 | | | | | | | | |
| H3LVCY- | 96.10190133 | 78.10041349 | 16.565442 | 1.943719774 | 3.390424731 | 0.72 | 100 | 100 |
| NA12878- | | | | | | | | |
| SC12 | | | | | | | | |
| H3LVCY- | 95.55288996 | 77.23723571 | 17.58099681 | 1.884855245 | 3.296912234 | 0.72 | 100 | 100 |
| NA12878- | | | | | | | | |
| SC13 | | | | | | | | |
| H3LVCY- | 95.57108887 | 74.23684733 | 20.42053294 | 1.807321858 | 3.535297877 | 0.77 | 100 | 100 |
| NA12878- | | | | | | | | |
| SC14 | | | | | | | | |
| H3LVCY- | 94.81590154 | 75.05957823 | 19.36479863 | 2.023411622 | 3.552211514 | 0.83 | 100 | 100 |
| NA12878- | | | | | | | | |
| SC15 | | | | | | | | |
| H3LVCY- | 94.41602834 | 71.86653744 | 22.21857986 | 2.3174938 | 3.5973889 | 0.77 | 100 | 100 |
| NA12878- | | | | | | | | |
| SC16 | | | | | | | | |
| H3LVCY- | 95.16234666 | 73.55793922 | 19.82861084 | 3.033059913 | 3.580390028 | 0.75 | 100 | 100 |
| NA12878- | | | | | | | | |
| SC2 | | | | | | | | |
| H3LVCY- | 95.68830354 | 69.88109705 | 24.29647104 | 2.473371935 | 3.349059976 | 0.76 | 100 | 100 |
| NA12878- | | | | | | | | |
| SC3 | | | | | | | | |
| H3LVCY- | 96.28690827 | 73.79777633 | 20.46857885 | 2.192646588 | 3.540998226 | 0.72 | 100 | 100 |
| NA12878- | | | | | | | | |
| SC4 | | | | | | | | |
| H3LVCY- | 95.9857469 | 74.53199872 | 20.11819827 | 2.053029496 | 3.296773507 | 0.72 | 100 | 100 |
| NA12878- | | | | | | | | |
| SC5 | | | | | | | | |
| H3LVCY- | 95.93023256 | 64.62272931 | 26.45986401 | 4.780623474 | 4.136783208 | 0.7 | 100 | 100 |
| NA12878- | | | | | | | | |
| SC6 | | | | | | | | |
| H3LVCY- | 95.64956643 | 65.88557314 | 27.85677727 | 2.788348132 | 3.469301466 | 0.75 | 100 | 100 |
| NA12878- | | | | | | | | |
| SC7 | | | | | | | | |
| H3LVCY- | 95.84038866 | 73.90085531 | 20.92069321 | 1.981998119 | 3.196453361 | 0.77 | 100 | 100 |
| NA12878- | | | | | | | | |
| SC8 | | | | | | | | |
| H3LVCY- | 95.94740092 | 80.24828218 | 15.03435639 | 1.495190105 | 3.222171324 | 0.75 | 100 | 100 |
| NA12878- | | | | | | | | |
| SC9 | | | | | | | | |
| H3LVVH- | 92.99936327 | 80.44018695 | 14.42319379 | 1.67611345 | 3.460505817 | 0.73 | 100 | 100 |
| NA12878- | | | | | | | | |
| SC1 | | | | | | | | |
| H3LVVH- | 93.43108158 | 69.82641494 | 20.66290836 | 5.086893922 | 4.423782775 | 0.73 | 100 | 100 |
| NA12878- | | | | | | | | |
| SC2 | | | | | | | | |
| H3LVVH- | 93.40679431 | 77.72108324 | 16.61930806 | 1.794534918 | 3.865073788 | 0.76 | 100 | 100 |
| NA12878- | | | | | | | | |
| SC3 | | | | | | | | |
| H3LVVH- | 93.06020521 | 77.57111977 | 15.8469061 | 2.633252032 | 3.948722098 | 0.7 | 100 | 100 |
| NA12878- | | | | | | | | |
| SC4 | | | | | | | | |
| H3LVVH- | 94.49338821 | 77.85689494 | 15.75670246 | 2.717797154 | 3.668605446 | 0.75 | 100 | 100 |
| NA12878- | | | | | | | | |
| SC5 | | | | | | | | |
| H3LVVH- | 92.51478529 | 70.13711105 | 23.40298507 | 2.933468252 | 3.526435619 | 0.75 | 100 | 100 |
| NA12878- | | | | | | | | |
| SC6 | | | | | | | | |
| H3LVVH- | 92.8455563 | 79.50854445 | 14.45975786 | 2.213616734 | 3.818080952 | 0.75 | 100 | 100 |
| NA12878- | | | | | | | | |
| SC7 | | | | | | | | |
| H3LVVH- | 92.19754692 | 82.85477732 | 11.27950968 | 2.105493141 | 3.760219864 | 0.77 | 100 | 100 |
| NA12878- | | | | | | | | |
| SC8 | | | | | | | | |
| H3LVVH- | 92.59625654 | 75.93017401 | 18.45465227 | 2.0354725 | 3.579701225 | 0.83 | 100 | 100 |
| NA12878- | | | | | | | | |
| SC9 | | | | | | | | |
| H3LVVH- | 91.8700165 | 77.73584082 | 15.52055153 | 3.00855223 | 3.735055415 | 0.79 | 100 | 100 |
| NA12878- | | | | | | | | |
| SC10 | | | | | | | | |
| H3LVVH- | 92.7763959 | 81.25638347 | 12.55561686 | 2.188281424 | 3.999718247 | 0.74 | 100 | 100 |
| NA12878- | | | | | | | | |
| SC11 | | | | | | | | |
| H3LVVH- | 93.16454828 | 76.31388895 | 17.23376597 | 3.112683903 | 3.339661185 | 0.69 | 100 | 100 |
| NA12878- | | | | | | | | |
| SC12 | | | | | | | | |
| H3LVVH- | 93.46493535 | 75.55579643 | 16.48149152 | 4.095170993 | 3.867541055 | 0.76 | 100 | 100 |
| NA12878- | | | | | | | | |
| SC13 | | | | | | | | |
| H3LVVH- | 92.810452 | 73.09440595 | 20.41354371 | 2.796016081 | 3.696034263 | 0.77 | 100 | 100 |
| NA12878- | | | | | | | | |
| SC14 | | | | | | | | |
| H3LVVH- | 91.67634104 | 68.03276155 | 24.94823057 | 3.148405708 | 3.870602174 | 0.74 | 100 | 100 |
| NA12878- | | | | | | | | |
| SC17 | | | | | | | | |
| H3LVVH- | 91.16315851 | 69.34624396 | 22.44811213 | 4.527359058 | 3.678284849 | 0.74 | 100 | 100 |
| NA12878- | | | | | | | | |
| SC18 | | | | | | | | |
| H3LVVH- | 90.59686133 | 75.04062115 | 18.10012977 | 3.430715041 | 3.42853404 | 0.72 | 100 | 100 |
| NA12878- | | | | | | | | |
| SC19 | | | | | | | | |
| H3LVVH- | 90.43034026 | 73.23524746 | 19.11088198 | 4.152125635 | 3.501744924 | 0.77 | 100 | 100 |
| NA12878- | | | | | | | | |
| SC20 | | | | | | | | |
| H3LVVH- | 91.67025434 | 75.71434441 | 18.11487028 | 2.530761488 | 3.640023829 | 0.74 | 100 | 100 |
| NA12878- | | | | | | | | |
| SC21 | | | | | | | | |
| H3LVVH- | 90.33082209 | 69.00846347 | 24.01108484 | 3.501995485 | 3.478456254 | 0.71 | 100 | 100 |
| NA12878- | | | | | | | | |
| SC22 | | | | | | | | |
| H3LVVH- | 90.6567299 | 81.64371941 | 13.16649279 | 1.741393344 | 3.448394456 | 0.79 | 100 | 100 |
| NA12878- | | | | | | | | |
| SC24 | | | | | | | | |
| H3LVVH- | 92.49529949 | 70.47752217 | 22.44010165 | 3.689893576 | 3.392482605 | 0.8 | 100 | 100 |
| NA12878- | | | | | | | | |
| SC25 | | | | | | | | |
| H3LVVH- | 91.9295082 | 75.53324721 | 17.98100723 | 2.637884959 | 3.847860601 | 0.75 | 100 | 100 |
| NA12878- | | | | | | | | |
| SC26 | | | | | | | | |
| H3LVVH- | 92.26303337 | 74.89687081 | 18.00333698 | 3.560132251 | 3.539659955 | 0.78 | 100 | 100 |
| NA12878- | | | | | | | | |
| SC27 | | | | | | | | |
| H3LVVH- | 92.65044449 | 76.60421191 | 16.73955861 | 2.958436443 | 3.697793041 | 0.79 | 100 | 100 |
| NA12878- | | | | | | | | |
| SC28 | | | | | | | | |
| H3LVVH- | 91.64109219 | 71.07149777 | 21.19313656 | 3.948416543 | 3.786949127 | 0.77 | 100 | 100 |
| NA12878- | | | | | | | | |
| SC29 | | | | | | | | |
| H3LVVH- | 94.1911707 | 80.76028939 | 13.4854656 | 1.943499042 | 3.810745969 | 0.77 | 100 | 100 |
| NA12878- | | | | | | | | |
| SC30 | | | | | | | | |
| H3LVVH- | 90.62272884 | 64.54046118 | 28.85944263 | 2.926063736 | 3.67403245 | 0.77 | 100 | 100 |
| NA12878- | | | | | | | | |
| SC31 | | | | | | | | |
| H3LVVH- | 90.68130065 | 77.06972239 | 16.11673296 | 2.926567878 | 3.886976773 | 0.77 | 100 | 100 |
| NA12878- | | | | | | | | |
| SC32 | | | | | | | | |
| H3LVVH- | 87.39235013 | 36.59589592 | 53.74813142 | 7.004944544 | 2.65102811 | 0.62 | 100 | 100 |
| NA12878- | | | | | | | | |
| SC33 | | | | | | | | |
| H3LVVH- | 93.44446683 | 76.98629309 | 17.44030294 | 2.141137845 | 3.432266121 | 0.73 | 100 | 100 |
| NA12878- | | | | | | | | |
| SC34 | | | | | | | | |
| H3LVVH- | 90.86897488 | 63.70722807 | 29.82920097 | 3.265852898 | 3.19771806 | 0.72 | 100 | 100 |
| NA12878- | | | | | | | | |
| SC35 | | | | | | | | |
| H3LVVH- | 91.15575536 | 70.5102156 | 23.0353313 | 3.092899876 | 3.361553223 | 0.76 | 100 | 100 |
| NA12878- | | | | | | | | |
| SC36 | | | | | | | | |
| H3LVVH- | 92.35858119 | 75.48073831 | 19.23167621 | 2.082663408 | 3.204922074 | 0.73 | 100 | 100 |
| NA12878- | | | | | | | | |
| SC37 | | | | | | | | |
| H3LVVH- | 91.77375806 | 70.31330161 | 22.26192793 | 4.01578007 | 3.408990394 | 0.76 | 100 | 100 |
| NA12878- | | | | | | | | |
| SC38 | | | | | | | | |
| H3LVVH- | 93.44890663 | 76.14694532 | 18.41928532 | 2.249285822 | 3.184483536 | 0.78 | 100 | 100 |
| NA12878- | | | | | | | | |
| SC39 | | | | | | | | |
| H3LVVH- | 90.681248 | 65.79994747 | 27.07638847 | 3.916398974 | 3.207265087 | 0.74 | 100 | 100 |
| NA12878- | | | | | | | | |
| SC40 | | | | | | | | |
| H3MG2V- | 88.85084156 | 57.50371899 | 35.569845 | 3.870627189 | 3.05580882 | 0.62 | 100 | 100 |
| HBRR | | | | | | | | |
| H3MG2V- | 90.99376252 | 57.45461082 | 35.63365091 | 3.777637965 | 3.134100306 | 0.66 | 100 | 100 |
| HBRR | | | | | | | | |
| H3MG2V- | 88.56466643 | 59.98027395 | 33.17599461 | 3.534586005 | 3.309145439 | 0.63 | 100 | 100 |
| HBRR | | | | | | | | |
| H3MG2V- | 91.498856 | 73.06526661 | 21.11760826 | 2.344321038 | 3.472804093 | 0.76 | 100 | 100 |
| UHRR | | | | | | | | |
| H3MG2V- | 92.55285871 | 74.02856691 | 20.43483614 | 2.108216503 | 3.428380446 | 0.71 | 100 | 100 |
| UHRR | | | | | | | | |
| H3MG2V- | 90.73710342 | 72.50092935 | 21.54148448 | 2.495087713 | 3.462498451 | 0.73 | 100 | 100 |
| UHRR | | | | | | | | |
| H3MG2V- | 91.50540153 | 56.60039779 | 36.45181505 | 3.864422789 | 3.083364364 | 0.69 | 100 | 100 |
| HBRR | | | | | | | | |
| H3MG2V- | 91.58824659 | 55.88162126 | 37.212645 | 3.87347627 | 3.03225747 | 0.61 | 100 | 100 |
| HBRR | | | | | | | | |
| H3MG2V- | 88.8888888 | 69.33333333 | 26.66666667 | 0 | 4 | NA | 100 | 100 |
| HBRR | | | | | | | | |
| H3MG2V- | 92.02597478 | 73.87717773 | 20.3448151 | 2.4258394 | 3.352167771 | 0.73 | 100 | 100 |
| UHRR | | | | | | | | |
| H3MG2V- | 93.20943127 | 73.29281903 | 20.77128309 | 2.420395494 | 3.515502389 | 0.74 | 100 | 100 |
| UHRR | | | | | | | | |
| H3MG2V- | 91.48551728 | 71.01859058 | 23.15607436 | 2.543882404 | 3.281452659 | 0.73 | 100 | 100 |
| UHRR | | | | | | | | |
| H3MG2V- | 91.46300754 | 58.27754174 | 34.90193554 | 3.810995236 | 3.009527488 | 0.69 | 100 | 100 |
| HBRR | | | | | | | | |
| H3MG2V- | 89.42586272 | 57.94876625 | 35.35411565 | 3.828404596 | 2.868713505 | 0.65 | 100 | 100 |
| HBRR | | | | | | | | |
| H3MG2V- | 90.29439462 | 56.57066437 | 36.79132636 | 3.853465157 | 2.784544109 | 0.65 | 100 | 100 |
| HBRR | | | | | | | | |
| H3MG2V- | 92.66525848 | 78.22083824 | 16.3331089 | 2.014812321 | 3.431240532 | 0.74 | 100 | 100 |
| UHRR | | | | | | | | |
| H3MG2V- | 93.37282616 | 79.41502044 | 14.98126703 | 2.012091281 | 3.591621253 | 0.79 | 100 | 100 |
| UHRR | | | | | | | | |
| H3MG2V- | 89.52454449 | 78.9056063 | 15.53197206 | 1.999820885 | 3.562600752 | 0.75 | 100 | 100 |
| UHRR | | | | | | | | |
| H3MG2V- | 92.75075314 | 59.08801311 | 34.14125785 | 3.448621098 | 3.322107946 | 0.65 | 100 | 100 |
| HBRR | | | | | | | | |
| H3MG2V- | 91.57071839 | 54.0848784 | 39.02612881 | 3.830028589 | 3.058964198 | 0.68 | 100 | 100 |
| HBRR | | | | | | | | |
| H3MG2V- | 93.17580285 | 59.09166267 | 34.1590681 | 3.512063174 | 3.237206056 | 0.61 | 100 | 100 |
| HBRR | | | | | | | | |
| H3MG2V- | 95.92254898 | 69.29173893 | 25.2373291 | 2.256628847 | 3.214303127 | 0.77 | 100 | 100 |
| UHRR | | | | | | | | |
| H3MG2V- | 94.82280229 | 73.80852183 | 20.68986248 | 2.183813031 | 3.31780266 | 0.7 | 100 | 100 |
| UHRR | | | | | | | | |
| H3MG2V- | 94.83838013 | 74.80545861 | 19.5625778 | 2.237512314 | 3.394451275 | 0.75 | 100 | 100 |
| UHRR | | | | | | | | |
| H3MGFK- | 91.80905811 | 60.44075909 | 33.00239301 | 3.188825199 | 3.368022706 | 0.76 | 100 | 100 |
| HBRR | | | | | | | | |
| H3MGFK- | 91.08096167 | 57.28144298 | 35.58325467 | 3.713223169 | 3.422079178 | 0.74 | 100 | 100 |
| HBRR | | | | | | | | |
| H3MGFK- | 91.44702137 | 56.71079624 | 36.50049884 | 3.581557501 | 3.207147421 | 0.63 | 100 | 100 |
| HBRR | | | | | | | | |
| H3MGFK- | 92.46856617 | 77.38074655 | 17.04896932 | 1.888459839 | 3.681824294 | 0.81 | 100 | 100 |
| UHRR | | | | | | | | |
| H3MGFK- | 93.36501724 | 76.47732415 | 17.87287486 | 1.859872376 | 3.789928616 | 0.77 | 100 | 100 |
| UHRR | | | | | | | | |
| H3MGFK- | 92.61442593 | 74.36628286 | 19.93656311 | 2.151640235 | 3.545513795 | 0.79 | 100 | 100 |
| UHRR | | | | | | | | |
| H3MGFK- | 93.28070099 | 57.51408474 | 35.51521819 | 3.810381038 | 3.160316032 | 0.71 | 100 | 100 |
| HBRR | | | | | | | | |
| H3MGFK- | 92.86267757 | 56.40908899 | 36.81463682 | 3.703047932 | 3.073226255 | 0.75 | 100 | 100 |
| HBRR | | | | | | | | |
| H3MGFK- | 93.17513332 | 56.92565133 | 36.38328876 | 3.654181812 | 3.036878093 | 0.69 | 100 | 100 |
| HBRR | | | | | | | | |
| H3MGFK- | 95.05033873 | 76.23972766 | 18.12701923 | 2.077957747 | 3.555295359 | 0.79 | 100 | 100 |
| UHRR | | | | | | | | |
| H3MGFK- | 94.64889544 | 75.80159218 | 18.51270026 | 2.115065 | 3.570642564 | 0.81 | 100 | 100 |
| UHRR | | | | | | | | |
| H3MGFK- | 95.19731362 | 75.66177402 | 18.84052569 | 1.885990742 | 3.611709543 | 0.77 | 100 | 100 |
| UHRR | | | | | | | | |
| H3MGFK- | 93.32688588 | 56.71017461 | 36.46856667 | 3.803762974 | 3.017495748 | 0.64 | 100 | 100 |
| HBRR | | | | | | | | |
| H3MGFK- | 91.37230469 | 54.64248918 | 38.45703688 | 3.800535751 | 3.099938183 | 0.72 | 100 | 100 |
| HBRR | | | | | | | | |
| H3MGFK- | 92.63446434 | 55.45622251 | 37.80248583 | 3.772151687 | 2.969139976 | 0.68 | 100 | 100 |
| HBRR | | | | | | | | |
| H3MGFK- | 94.71336676 | 73.94825928 | 20.5354717 | 2.139096336 | 3.377172692 | 0.77 | 100 | 100 |
| UHRR | | | | | | | | |
| H3MGFK- | 94.85190124 | 75.61821925 | 18.89009693 | 2.01253788 | 3.479145944 | 0.77 | 100 | 100 |
| UHRR | | | | | | | | |
| H3MGFK- | 95.02232729 | 75.19298134 | 19.1206074 | 2.024124649 | 3.662286611 | 0.73 | 100 | 100 |
| UHRR | | | | | | | | |
| H3MGFK- | 86.98377238 | 57.75663757 | 35.22456729 | 3.870533517 | 3.148261625 | 0.66 | 100 | 100 |
| HBRR | | | | | | | | |
| H3MGFK- | 86.89606936 | 56.68376614 | 36.44883131 | 3.714645003 | 3.15275754 | 0.65 | 100 | 100 |
| HBRR | | | | | | | | |
| H3MGFK- | 90.92354467 | 56.60951981 | 36.20467852 | 3.98540261 | 3.200399065 | 0.68 | 100 | 100 |
| HBRR | | | | | | | | |
| H3MGFK- | 88.52459016 | 89.81549481 | 5.722834217 | 0.854073167 | 3.607597806 | 0.72 | 100 | 100 |
| NA12878- | | | | | | | | |
| 100pg | | | | | | | | |
| H3MGFK- | 82.68085046 | 89.57935463 | 5.83817374 | 0.922233547 | 3.660238079 | 0.72 | 100 | 100 |
| NA12878- | | | | | | | | |
| 100pg | | | | | | | | |
| H3MGFK- | 96.29308394 | 82.18995968 | 12.40073251 | 1.399869698 | 4.009438115 | 0.79 | 100 | 100 |
| NA12878- | | | | | | | | |
| SC1 | | | | | | | | |
| H3MGFK- | 86.43049163 | 82.26344976 | 11.15659689 | 2.481394381 | 4.098558972 | 0.78 | 100 | 100 |
| NA12878- | | | | | | | | |
| SC10 | | | | | | | | |
| H3MGFK- | 92.99330094 | 81.56411566 | 11.79627788 | 2.38255244 | 4.257054022 | 0.73 | 100 | 100 |
| NA12878- | | | | | | | | |
| SC11 | | | | | | | | |
| H3MGFK- | 95.72530958 | 84.5052095 | 10.45400293 | 1.338945177 | 3.701842396 | 0.77 | 100 | 100 |
| NA12878- | | | | | | | | |
| SC12 | | | | | | | | |
| H3MGFK- | 79.29047459 | 76.40541769 | 17.42862943 | 2.350177399 | 3.815775481 | 0.78 | 100 | 100 |
| NA12878- | | | | | | | | |
| SC13 | | | | | | | | |
| H3MGFK- | 90.12475022 | 82.25136054 | 12.2633368 | 1.639690731 | 3.845611934 | 0.71 | 100 | 100 |
| NA12878- | | | | | | | | |
| SC14 | | | | | | | | |
| H3MGFK- | 95.12984193 | 78.85820513 | 15.74993902 | 1.631015368 | 3.76084049 | 0.78 | 100 | 100 |
| NA12878- | | | | | | | | |
| SC15 | | | | | | | | |
| H3MGFK- | 91.63549813 | 81.21182239 | 12.96372385 | 1.65265109 | 4.171802668 | 0.73 | 100 | 100 |
| NA12878- | | | | | | | | |
| SC16 | | | | | | | | |
| H3MGFK- | 89.77030715 | 78.05312851 | 16.29729075 | 1.853115303 | 3.796465436 | 0.75 | 100 | 100 |
| NA12878- | | | | | | | | |
| SC2 | | | | | | | | |
| H3MGFK- | 86.13426675 | 82.70995122 | 12.10488316 | 1.553172381 | 3.631993238 | 0.78 | 100 | 100 |
| NA12878- | | | | | | | | |
| SC3 | | | | | | | | |
| H3MGFK- | 90.71849979 | 74.4109307 | 18.83810812 | 2.940553195 | 3.810407984 | 0.77 | 100 | 100 |
| NA12878- | | | | | | | | |
| SC4 | | | | | | | | |
| H3MGFK- | 90.87209391 | 82.87345769 | 11.54750255 | 1.572825133 | 4.006214627 | 0.79 | 100 | 100 |
| NA12878- | | | | | | | | |
| SC5 | | | | | | | | |
| H3MGFK- | 91.5848376 | 86.01200383 | 8.227164977 | 1.587733898 | 4.173097292 | 0.76 | 100 | 100 |
| NA12878- | | | | | | | | |
| SC6 | | | | | | | | |
| H3MGFK- | 88.41693085 | 76.49431569 | 17.64785416 | 2.029227181 | 3.828602977 | 0.76 | 100 | 100 |
| NA12878- | | | | | | | | |
| SC7 | | | | | | | | |
| H3MGFK- | 92.60733124 | 76.63644506 | 15.44226516 | 3.356060468 | 4.565229313 | 0.73 | 100 | 100 |
| NA12878- | | | | | | | | |
| SC8 | | | | | | | | |
| H3MGFK- | 90.72247626 | 82.79735623 | 11.12994914 | 1.854991523 | 4.217703111 | 0.75 | 100 | 100 |
| NA12878- | | | | | | | | |
| SC9 | | | | | | | | |
| H3MGFK- | 79.33685638 | 75.05264906 | 19.30057586 | 2.159266671 | 3.487508411 | 0.77 | 100 | 100 |
| UHRR | | | | | | | | |
| H3MGFK- | 86.15003844 | 76.41109299 | 18.08448528 | 1.985060531 | 3.519361209 | 0.75 | 100 | 100 |
| UHRR | | | | | | | | |
| H3MGFK- | 86.29136833 | 75.68559551 | 18.75298722 | 1.941581221 | 3.619836052 | 0.71 | 100 | 100 |
| UHRR | | | | | | | | |

### Example 2: Analysis of SKBR3 Cells with PTA

Following the general methods of Example 1, SKBR3 tumor cells were picked from a suspension using a CellCelector™ instrument (ALS). Cells were deposited in a 96-well plate, or in a flat bottom strip tube. Cells in the flat bottom tube strip were imaged using CellTracker CMFDA staining. After cell lysis and amplification using PTA, amplicon yields were measured. Sizes of amplicons before and after library preparation were measured by gel staining. Performance metrics after sequencing are shown in Tables 1-2.

TABLE 1
Performance metrics for single SKBR3 cells

| Sample | PCT aligned | Chr. M | Preseq count |
|---|---|---|---|
| Single cell C5 | 0.994079 | 0.007178679 | 3512530229 |
| Single cell B6 | 0.993858 | 0.018429564 | 3480463721 |
| Single cell C7 | 0.993367 | 0.009323215 | 3493218511 |
| Single cell B8 | 0.994573 | 0.007614784 | 3534448748 |
| 5 cell B11 | 0.993976 | 0.012700483 | 3425303039 |
| 5 cell C11 | 0.994229 | 0.008341315 | 3619845236 |

TABLE 2
Aggregate performance for PTA analysis of SKBR3 single cells

| QC Metric | Value |
|---|---|
| PreSeq Count | 3.50 (±0.02) × 10⁹ |
| Alignment | 0.978 ± 0.001 |
| Chimeras | 0.106 ± 0.007 |
| ChrM | 0.01 ± 0.005 |

### Example 3: Generating Sequencing Data from Individually Selected Cells

NA12878 cells were isolated as single cells. Genomic and transcriptomic libraries were prepared by the multiomics step depicted in FIG. 1. First, the cytosol was lysed. Then the mRNA transcriptome was converted to cDNA using 1^{st} strand synthesis. Next, nuclear lysis occurred. Whole genome amplification via PTA occurred. The transcriptome cDNA and genomic DNA were then isolated. The cDNA was pre-amplified via PTA and a library was prepared for NGS of the transcriptomic library. Likewise, library prep of the PTA-amplified genomic DNA occurred and the genomic library was analyzed via NGS.

Yield of the amplified single-cell genomes and pre-amplified single-cell transcriptomes was measured. High quality genomic and transcriptomic sequencing data was produced from individually selected single cells, as can be seen in Tables 3-4.

TABLE 3
Amplified Single-cell genomes

| | PreSeq | % | % | % | % |
|---|---|---|---|---|---|
| sample_name | Count | chrM | Chimeras | Aligned | Error |
| SC1 | 3787323487 | 0.23 | 14.14 | 99.93 | 0.92 |
| SC10 | 3932827674 | 0.5 | 14.48 | 99.93 | 0.92 |
| SC11 | 4002773775 | 0.34 | 14.1 | 99.93 | 0.91 |
| SC12 | 3847952276 | 0.74 | 14.31 | 99.93 | 0.91 |
| SC13 | 4013224655 | 0.43 | 14.21 | 99.93 | 0.92 |
| SC14 | 3800485108 | 0.3 | 14.77 | 99.93 | 0.91 |
| SC16 | 3707159902 | 0.34 | 13.89 | 99.94 | 0.91 |
| SC17 | 3705102478 | 0.27 | 13.88 | 99.94 | 0.91 |
| SC18 | 3818941981 | 0.58 | 14.5 | 99.93 | 0.92 |
| SC19 | 3985623868 | 0.41 | 14.04 | 99.93 | 0.91 |
| SC2 | 3944785617 | 0.46 | 13.78 | 99.94 | 0.92 |
| SC20 | 3796745630 | 0.3 | 14.07 | 99.93 | 0.91 |
| SC21 | 3987691489 | 0.34 | 14.43 | 99.93 | 0.92 |
| SC22 | 3994750838 | 0.32 | 14.82 | 99.93 | 0.92 |
| SC23 | 4039019959 | 0.3 | 13.83 | 99.93 | 0.92 |
| SC24 | 4074078618 | 0.28 | 13.65 | 99.93 | 0.92 |
| SC3 | 3976725567 | 0.62 | 15.26 | 99.92 | 0.93 |
| SC4 | 4039929411 | 0.15 | 14.55 | 99.93 | 0.92 |
| SC5 | 3838848593 | 0.39 | 15.1 | 99.93 | 0.92 |
| SC6 | 3943920212 | 0.34 | 14.67 | 99.93 | 0.92 |
| SC7 | 3977255219 | 0.59 | 14.63 | 99.93 | 0.92 |
| SC9 | 3915034110 | 0.33 | 13.52 | 99.93 | 0.92 |

TABLE 4
Pre-amplified Single-cell transcriptomes

| | PreSeq | % | % | % | % |
|---|---|---|---|---|---|
| | Count | chrM | Chimeras | Aligned | Error |
| SC1 | 83682332 | 13.49 | 43.5 | 95.96 | 0.33 |
| SC10 | 20902483 | 19.45 | 41.09 | 95.88 | 0.33 |
| SC11 | 69281108 | 4.72 | 43.51 | 95.54 | 0.33 |
| SC12 | 64480167 | 9.21 | 39.39 | 95.82 | 0.32 |
| SC13 | 93567834 | 6.83 | 43.82 | 95.54 | 0.33 |
| SC14 | 781538977 | 3.53 | 45.07 | 96.41 | 0.32 |
| SC15 | 14701565 | 9.67 | 40.74 | 95.56 | 0.35 |
| SC16 | 41850181 | 4.91 | 42.71 | 95 | 0.34 |
| SC17 | 31504203 | 7.31 | 43.11 | 94.73 | 0.32 |
| SC18 | 39232684 | 18.3 | 42.09 | 95.69 | 0.32 |
| SC19 | 34583159 | 17.84 | 41.66 | 95.94 | 0.32 |
| SC2 | 62447205 | 8.2 | 41.42 | 95.67 | 0.34 |
| SC20 | 27678390 | 13.05 | 38.97 | 95.95 | 0.33 |
| SC21 | 28237879 | 16.52 | 39.9 | 96.04 | 0.33 |
| SC23 | 303712331 | 4.47 | 45.77 | 95.87 | 0.32 |
| SC24 | 37394199 | 6.78 | 39.75 | 95.31 | 0.32 |
| SC3 | 42282615 | 11.94 | 40.04 | 95.6 | 0.32 |
| SC4 | 41094986 | 5.6 | 40.7 | 95.24 | 0.32 |
| SC5 | 30333087 | 10.5 | 43.11 | 95.53 | 0.32 |
| SC6 | 20262607 | 3.92 | 41.83 | 95.83 | 0.34 |
| SC7 | 42515045 | 5.53 | 44.19 | 95.65 | 0.34 |
| SC9 | 29230215 | 9.95 | 43.26 | 94.97 | 0.32 |

### Example 4: Single Cell Genomic Analysis Reveals Diversity in Oncology

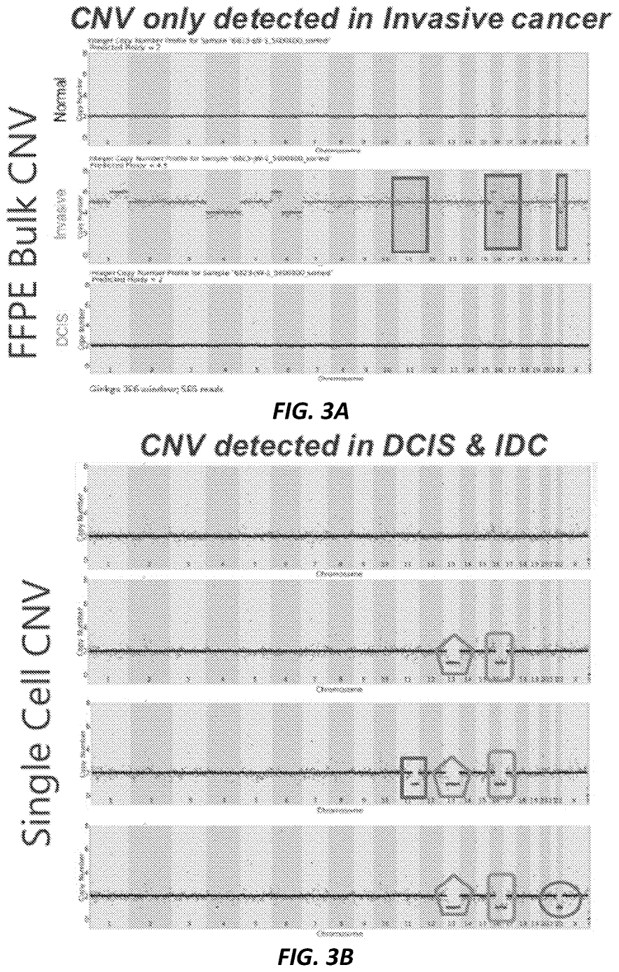

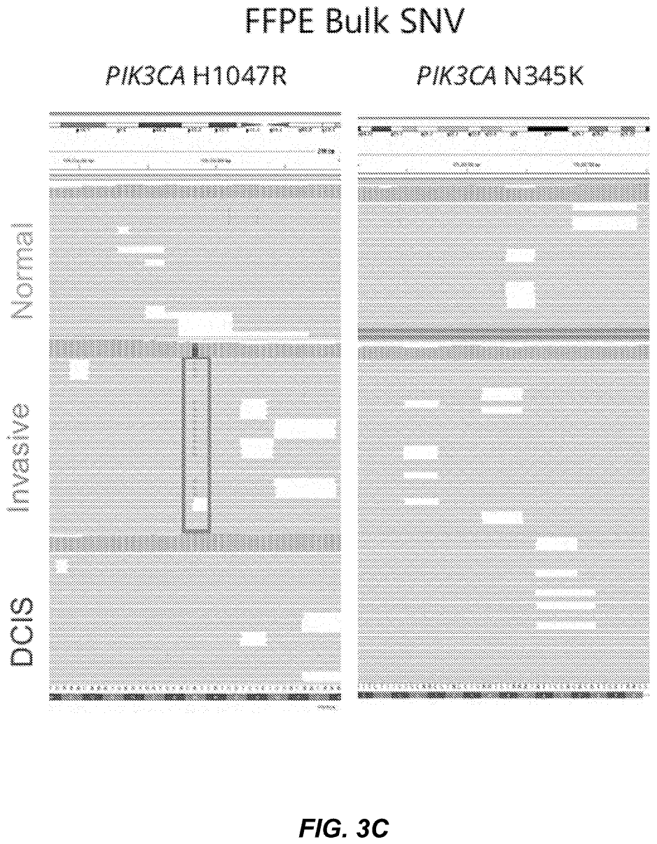

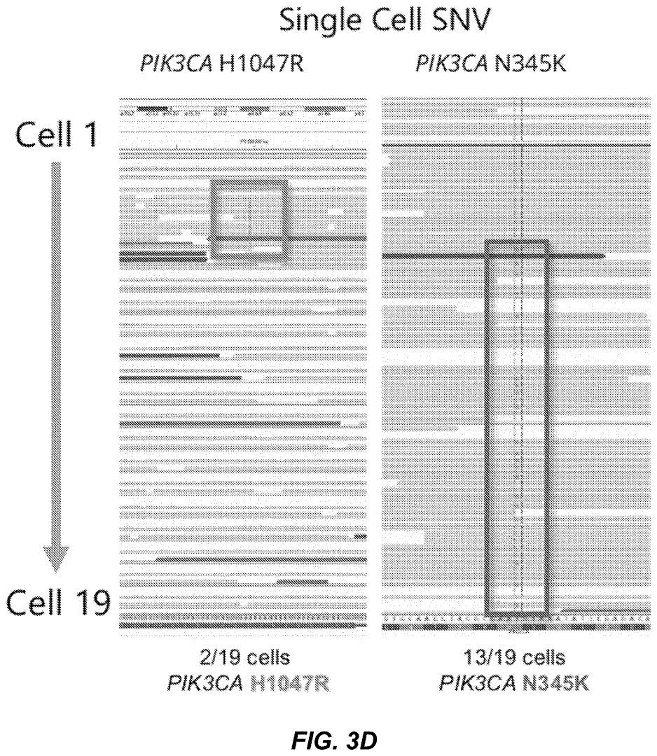

Differences in genome diversity detected in single cell sequencing versus bulk sequencing. In bulk sequencing of three primary patient samples, CNV were only detected in invasive cancer, as depicted in FIG. 3A. However, single cell sequencing detected CNV in both DCIS and IDC, as depicted in FIG. 3B.

Similar results were also seen in SNV. In bulk sequencing, only the PIK3CA H1047R mutation was detected (FIG. 3C). In single cell sequencing, PIK3CA N345K was detected in addition to PIK3CA H1047R (FIG. 3D).

### Example 5: Single-Cell Sequencing from Spatially Selected Cells

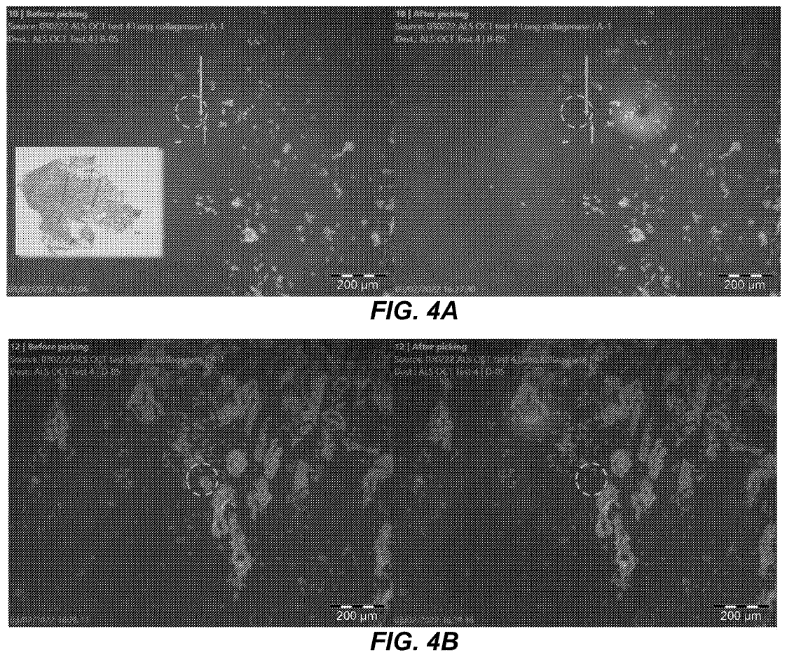

Single cells or small cell clusters were isolated from OCT tissue sections and selected for sequencing. FIG. 4A depicts the isolation of a single cell. In the left panel, two cells are seen within the image. A single cell was selected, as can be seen in the left panel, where only one cell remains within the circle. The spatial information of the selected cell is retained throughout analysis and can be used in later analysis. Likewise, a cluster of cells was isolated, as depicted in FIG. 4B.

Genomic DNA was isolated from the single cells or the cell clusters and amplified using PTA in high yield. Next generation sequencing was performed. Libraries created from the single-cells and cell clusters were the correct size fraction and yield. As depicted in Table 5, 11/15 cells passed quality control requirements for deep sequencing.

TABLE 5

| | PreSeq | % | % | % | % |
|---|---|---|---|---|---|
| sample_name | Count | chrM | Chimeras | Aligned | Error |
| SC03 | 3496691317 | 7.52 | 24.11 | 99.87 | 1.02 |
| SC04 | 2813526412 | 4.5 | 23.33 | 99.86 | 1.01 |
| SC08 | 3895936729 | 0.83 | 16.81 | 99.91 | 1.02 |
| SC20 | 2999615855 | 4.16 | 21.64 | 99.9 | 1.01 |
| SC06 | 2750806683 | 32.83 | 31.98 | 99.88 | 1.05 |
| SC07 | 3482785780 | 5.05 | 21.62 | 99.9 | 1.01 |
| SC09 | 3683766263 | 0.9 | 18.67 | 99.9 | 1.01 |
| SC10 | 3173333477 | 5.88 | 24.43 | 99.86 | 1.02 |
| SC11 | 3856625338 | 3.52 | 22.14 | 99.9 | 1.02 |
| SC12 | 3959193531 | 0.73 | 15.85 | 99.92 | 1.01 |
| SC15 | 3797430703 | 4.7 | 23.04 | 99.89 | 1.01 |
| SC17 | 3731576158 | 8.5 | 26.06 | 99.86 | 1.03 |
| SC19 | 2448233265 | 2.03 | 22.83 | 99.86 | 1.01 |
| SC21 | 3844158736 | 0.66 | 18.31 | 99.91 | 1.01 |
| SC24 | 3800253585 | 1.05 | 18.99 | 99.9 | 1 |

### Example 6: a Multiomic View of Ductal Carcinoma In Situ (DCIS)/Invasive Ductal Carcinoma

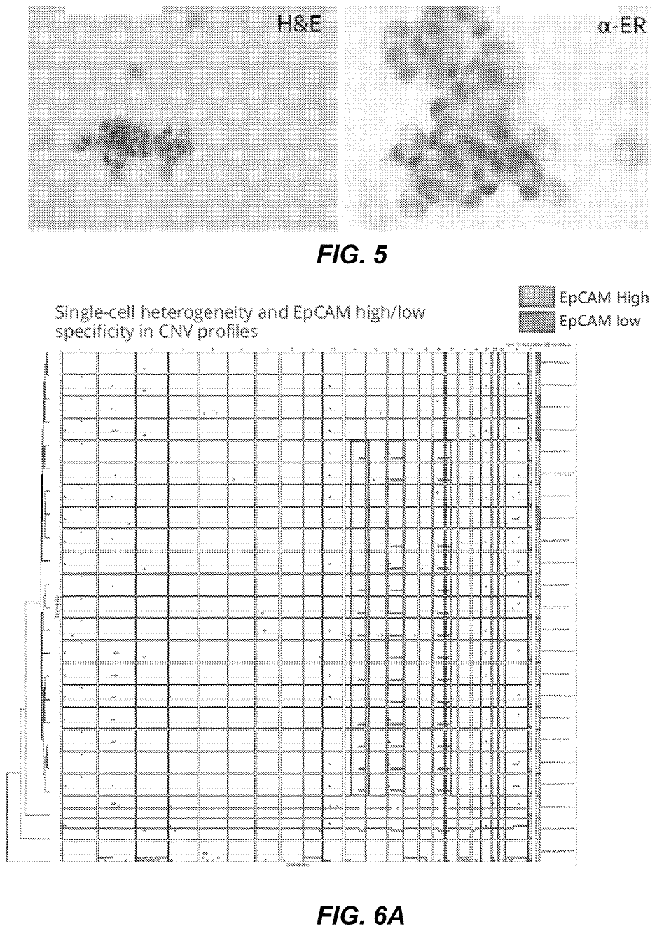

A 7 cm DCIS (grade II) and a 1.2 cm invasive cancer (grade I) were analyzed. The cancer was ER+PR+HER2—. Normal and tumor tissue were digested to single cells. The tissue was stained with H&E staining and formalin-fixed, paraffin embedded prior to genomic DNA isolation (FIG. 5). The transcriptome and genome were analyzed using the methods described in Example 5.

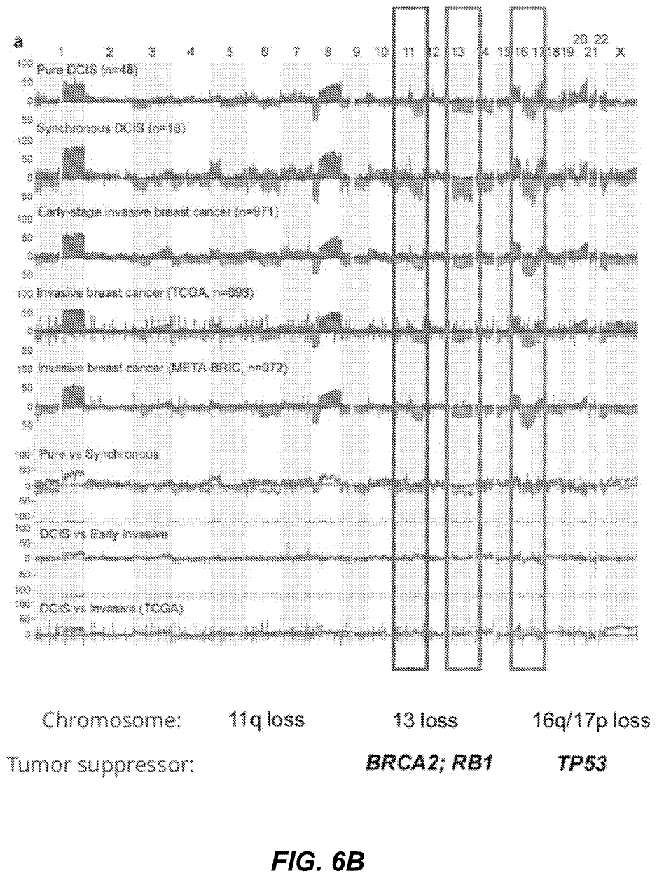

There was single-cell heterogeneity in CNV profiles of the primary breast cancer cells. Additionally, high and low EpCAM cells showed specificity in CNV profiles, as depicted in FIG. 6A. Known DCIS copy number alterations harbor prototypical tumor suppressor genes, as depicted in FIG. 6B.

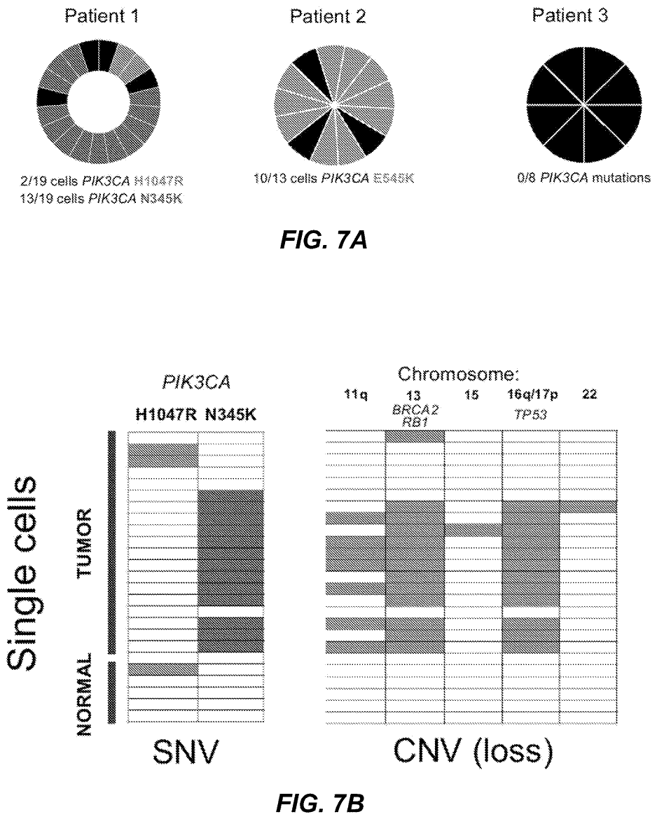

An analysis of SNV in primary breast cancer cells showed a variety of mutually exclusive single-cell oncogene PIK3CA mutations, as depicted in FIGS. 7A-7B. Patient 1 had 2/19 cells with a PIK3CA H1047R mutation and 13/19 cells with a PIK3CA N345K mutation. Patient 2 had 10/13 cells with a PIK3CA E545K mutation. Patient 3 had 0/8 cells with PIK3CA mutations. For patient 1, SNV and CNV were compared across the 19 cells analyzed. Heterogeneity was observed within single cells. However, some cells showed neither SNV nor CNV mutations (FIG. 7B).

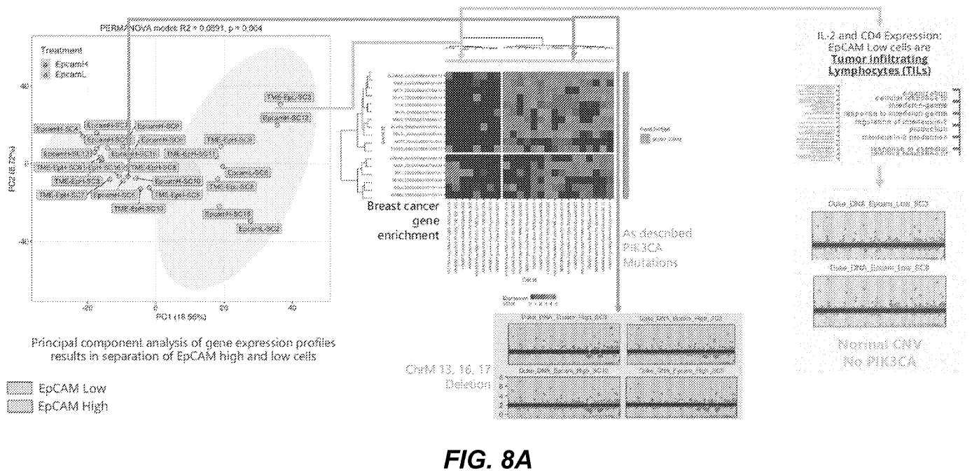

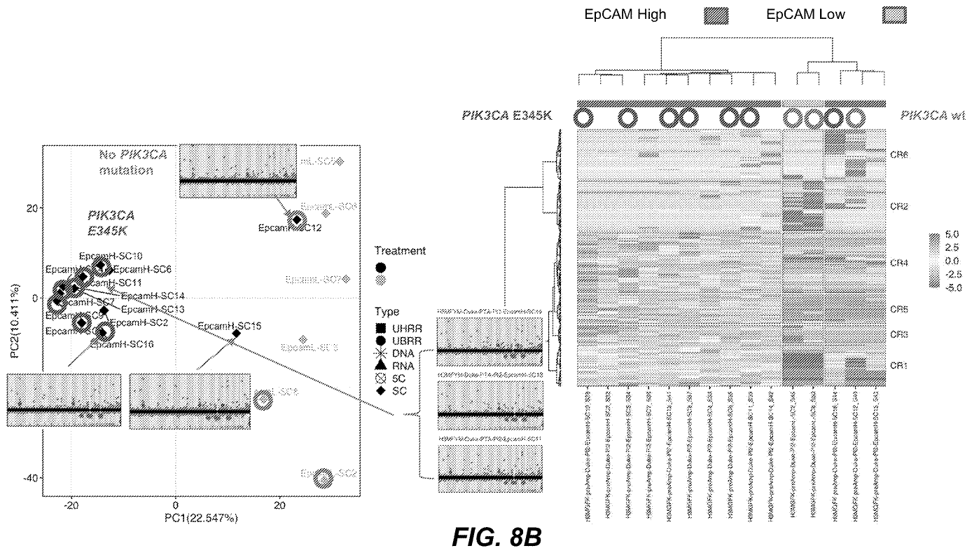

A principle component analysis of the gene expression profiles results in a separation of EpCAM high and low cells, as depicted in FIG. 8A. Clustering by genes enriched in breast cancer showed low levels of expression in the EpCAM low cells. IL-2 and CD4 expression suggests these cells are tumor infiltrating lymphocytes. Genomic and expression profiles for DCIS cells are shown in FIG. 8B.

The plasticity of the genome is significantly higher than found in the transcriptome and is the driver of cellular evolution. This method described transcriptional signatures that exposed the presence of tumor infiltrating lymphocytes in the tumor sample and guided interpretation of genotype. RNA mechanisms of resistance were jointly identified, including transcriptional bypass mechanisms in response to drug treatment. Unification of these DNA/RNA data identified candidate regulatory SNVs proximal to genes differentially influencing their expression between parental and resistant cells, thereby exposing novel genes and modes of drug resistance.

### Example 7: Combined PTA Multiomics with Spatial Fine Needle Aspirate (FNA) Sampling

A location on the human body is chosen, optionally comprising a suspicious mass such as breast, thyroid, lymph nodes, or skin masses. Other areas include pancreatic, gastrointestinal, esophageal, and tracheal areas, although with the appropriate imaging modality (e.g., ultrasound, MRI, fluoroscopy, CT scanning, or other method) any area of the body may be sampled. In some instances, patients are chosen who are unfit for conventional invasive biopsy methods.

One or more fine needle insertion vectors are then chosen to target specific areas or subareas of the location, including avoidance of other organs, infections, blood vessels or other underlying structures, as well as cosmetic factors. A needle is then inserted into the target regions or sub-regions of the location under guidance of an imaging modality (e.g., ultrasound). Needle sizes in some instances range from 25-18 gauge. The needle is aspirated to withdraw liquids or cells from the location, and the sample is associated (e.g., labeled, indexed and/or barcoded) with information such as patient identifier, insertion vector, target organs/suspected disease, or other label. Optionally, a core biopsy is also obtained.

Tumor cells may be acquired using fine needle aspiration. In some instances, the sample contains no more than 20% tumor cells, which in many cases frustrates current methods of spatial analysis and diagnosis. Any number of cells are in some instances analyzed, such as no more than 10,000, 5,000, 1,000, 500, 200, or 100 cells.

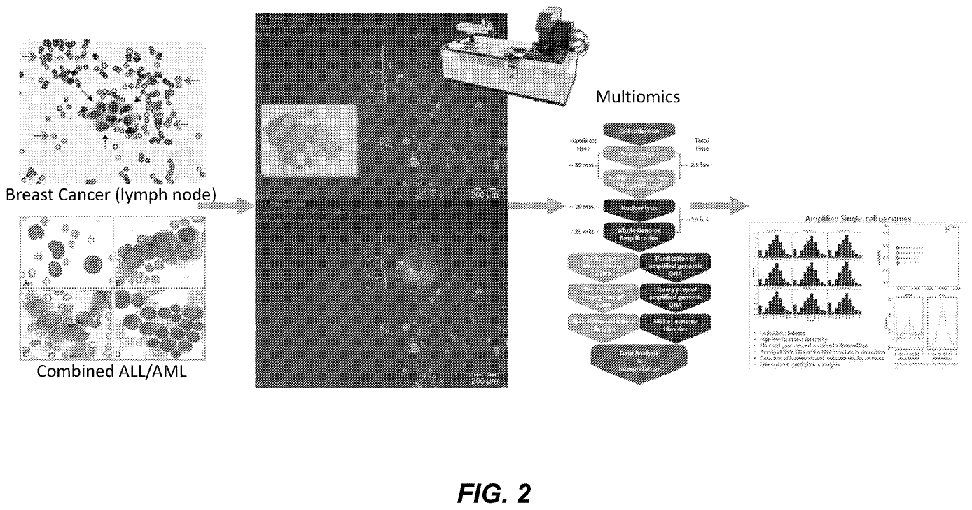

Cells obtained by FNA are then stained (e.g., diff-quik), single cells picked using one of the automated systems described herein, and single cells analyzed using the PTA multiomics methods described in Example 1 to identify potential genomic, transcriptome, proteome, methylome or other multiomics indicators which may identify a disease state. For example, individual cells are obtained from discrete needle locations in the sample and subjected to PTA amplification, library preparation, and sequencing to identify genomic variations, for example as illustrated in FIG. 2. Optionally, simultaneous transcriptome analysis is also conducted for each cell. Based on sequencing data obtained from the genome, a genetic diagnosis (or scoring) is made for individual cells as well as the tissue sample as a whole. This data is then combined with a two or three-dimensional model of the sampled location to produce a visual spatial map of single-cell data. In some instances, specific treatments such as surgical intervention or chemotherapy are recommended based on the data.

### Example 8: Combined PTA Multiomics with Low Cellularity Samples Obtained from FNA

Provided herein is a method for obtaining high quality multiomics using PTA from tissue samples obtained using FNA, even if they have low cellularity.

Cells are obtained from regions of tissue suspected to be cancerous using fine needle aspiration. Using FNA, the cells are obtained using a minimally invasive method and with high spatial accuracy of sample locations as the needle is guided using ultrasound. In some instances, the samples from FNA have low cellularity, which may traditionally affect the quality and accuracy of the results.

Here, the samples from FNA, including those with low cellularity, are analyzed using the PTA multiomics methods described in Example 1. PTA amplification is followed by library preparation and sequencing to identify genomic variants. After library generation and sequencing from PTA-created amplicons, more than 80%, 90%, 95%, 97%, 98%, or 99% of the genome is captured. In some instances, PTA results in high breadth of coverage, few replication errors, and low allelic bias, to accurately call single nucleotide variants (SNV) at the whole genome sequencing (WGS), whole exome sequencing (WES), and small-panel levels. The data is then combined with a 2D or 3D model of sample location to produce a visual map of single cell data.

The examples described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of spatial nucleic acid analysis comprising:
(a) providing a sample comprising a heterogeneous population of cells, wherein each cell has a unique location in the sample, and the sample was obtained using fine needle aspiration;
(b) isolating one or more cells from the population of cells, wherein the location of the one or more cells is recorded;
(c) amplifying DNA from one or more cells in the population of cells; and
(d) generating a genotype from the DNA, wherein the location of the one or more cells and the corresponding genotype are preserved.

2. A method of producing at least one map for visualizing different cell subtypes or cell states in a heterogeneous population of cells comprising:
(a) providing a sample comprising a heterogeneous population of cells, wherein each cell has a unique location in the sample, and the sample was obtained using fine needle aspiration;
(b) isolating one or more cells from the population of cells, wherein the location of the one or more cells is recorded;
(c) amplifying DNA from one or more cells in the population of cells;
(d) sequencing the amplified DNA from the one or more cells to generate a genotype; and
(e) generating at least one map which correlates the location of the one or more cells with the genotype.

3. The method of claim 1 or 2, wherein the fine needle aspiration further comprises a visualization method.

4. The method of claim 3, wherein the visualization method comprises one or more of ultrasound and CT scanning.

5. The method of any one of claims 1-4, wherein the method further comprises amplifying single cells from the population of cells.

6. The method of any one of claims 1-5, where the method further comprises reverse transcription of RNA in the one or more cells.

7. The method of any one of claims 1-6, wherein the population of cells comprises mammalian cells, microbial cells, fungal cells, or plant cells.

8. The method of any one of claims 1-7, wherein the population of cells comprises at least one cancer cell.

9. The method of claim 8, wherein no more than 20% of the population of cells are cancer cells.

10. The method of claim 8, wherein no more than 5% of the population of cells are cancer cells.

11. The method of claim 8, wherein no more than 1% of the population of cells are cancer cells.

12. The method of any one of claims 1-11, wherein no more than 20% of the population of cells are isolated.

13. The method of claim 12, wherein no more than 5% of the population of cells are isolated.

14. The method of claim 12, wherein no more than 1% of the population of cells are isolated.

15. The method of any one of claims 1-14, wherein the sample is an FFPE sample.

16. The method of any one of claims 1-15, wherein the sample is obtained from a tissue.

17. The method of claim 16, wherein the tissue comprises kidney, lung, breast, brain, esophagus, pancreas, colon, skin, bladder, ovary or prostate tissue.

18. The method of any one of claims 1-17, wherein the method further comprises scoring the one or more cells based on the genotype.

19. The method of any one of claims 1-18, wherein the method further comprises scoring the sample based on the genotypes of one or more single cells.

20. The method of any one of claims 1-19, wherein the one or more cells are isolated with a robotic device.

21. The method of claim 20, wherein the robotic device comprises a capillary fitting.

22. The method of claim 20, wherein the robotic device comprises an objective having a power of 1×-60×.

23. The method of any one of claims 1-22, wherein the one or more cells are contacted with a stain prior to isolation.

24. The method of claim 23, wherein the stain is configured to identify intercellular or intracellular targets.

25. The method of claim 23, wherein the stain comprises Diff-Quik stain.

26. The method of any one of claims 1-25, wherein the genotype provides for at least 95 percent alignment.

27. The method of any one of claims 1-26, wherein the genotype provides for a presequencing library complexity of at least 3.5×10⁸ counts.

28. The method of any one of claims 1-27, wherein the genotype provides for no more than 15% chimeras.

29. The method of any one of claims 1-28, wherein the genotype provides for no more than 5% mitochondrial chromosome reads.

30. The method of any one of claims 1-29, wherein amplifying DNA from one or more cells generates at least 100 ng of DNA.

31. The method of any one of claims 1-30, wherein amplifying comprises:
(a) contacting nucleic acids obtained from the isolated cells with at least one amplification primer, at least one nucleic acid polymerase, and a mixture of nucleotides, wherein the mixture of nucleotides comprises at least one terminator nucleotide which terminates nucleic acid replication by the polymerase, and
(b) amplifying the nucleic acids to generate a plurality of terminated amplification products, wherein the amplification proceeds by strand displacement replication and wherein the amplification is performed under conditions wherein the temperature varies by no more than 10 degrees C.

32. The method of claim 31, wherein the terminator is an irreversible terminator.

33. The method of claim 32, wherein the terminator nucleotide is selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids.

34. The method of claim 33, wherein the nucleotides with modification to the alpha group are alpha-thio dideoxynucleotides.

35. The method of any one of claims 31-34, wherein the terminator nucleotide comprises modifications of the r group of the 3′ carbon of the deoxyribose.

36. The method of any one of claims 31-35, wherein the terminator nucleotide is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof.

37. The method of any one of claims 31-36, wherein the plurality of terminated amplification products comprise an average of 1000-2000 bases in length.

38. The method of any one of claims 31-36, wherein at least some of the amplification products comprise a cell barcode, sample barcode, or spatial location barcode.

39. A system for spatial nucleic acid analysis comprising:
a sample comprising one or more cells, wherein the sample was obtained using fine needle aspiration;
a device comprising:
i. a cell collection module;
ii. an objective for visualizing single cells in the sample; and
iii. a robotic device configured to isolate single cells from the sample; and
at least one reaction chamber for isothermal amplification of nucleic acids from the one or more cells with one or more terminator nucleotides.

40. The system of claim 39, wherein the robotic device comprises a capillary fitting.

41. The system of claim 39 or 40, wherein the robotic device comprises an objective having a power of 1×-60×.

42. The system of any one of claims 39-41, wherein the system further comprises a computer interface.

43. The system of any one of claims 39-41, wherein the system further comprises a DNA sequencing instrument.

44. The system of any one of claims 39-43, wherein the cell collection module is configured to pick single cells, adherent colonies, or pick cells from media surface.
